(19) United States
(12) Patent Application Publication
(10) Pub. No.: US 2026/0250372 A9
(43) Pub. Date: Aug. 27, 2026

(54) METHODS FOR DETECTING OR TREATING ATHEROSCLEROSIS

(71) Applicant: City University of Hong Kong, Kowloon (HK)

(72) Inventors: Li Wang, Kowloon (HK); Yu Huang, Kowloon (HK)

(21) Appl. No.: 18/658,410

(22) Filed: May 8, 2024

(51) Int. Cl. C07K 16/24; G01N 33/68

(52) U.S. Cl. CPC C07K 16/241; G01N 33/6893; C07K 2317/34; C07K 2317/52; C07K 2317/54; C07K 2317/55; C07K 2317/565; C07K 2317/622; C07K 2317/92; G01N 2333/525; G01N 2800/32; G01N 2800/52

(57) ABSTRACT

The current disclosure provides sensitive diagnostic methods that do not require invasive angiography. It was found that TNFAIP2 was secreted into the sera of subjects with atherosclerosis. This novel biomarker can be used to detect early-stage atherosclerosis. Accordingly, aspects of the disclosure relate to a method for measuring a TNFAIP2 level comprising: obtaining a biological sample from a subject having, suspected of having, or at risk of having atherosclerosis; and measuring the TNFAIP2 level in a biological sample from the subject as compared to a control. Also disclosed is a method for treating or preventing atherosclerosis in a subject, the method comprising administering an effective amount of a treatment for atherosclerosis to the subject after measuring the TNFAIP2 level in a biological sample from the subject as compared to a control.

### SEQUENCE LISTING

The instant application contains a Sequence Listing which has been submitted in .xml format and is hereby incorporated by reference in its entirety. Said.xml copy, created on Mar. 29, 2024, is named “CTYUP0039 Sequence Listing” and is 18,339 bytes in size.

### TECHNICAL FIELD

Aspects of the disclosure include at least the fields of cell biology, molecular biology, and medicine, including therapeutic diagnosis. More particularly, it concerns methods and compositions involving prognosing, diagnosing, monitoring, and treating atherosclerosis.

### BACKGROUND

Cardiovascular diseases (CVD) are the leading cause of death globally, affecting over 10% of the population aged between 30-70 (Lee et al., 2019). Atherosclerotic vascular disease accounts for most CVD-related mortality. Atherosclerosis is a chronic inflammatory disease with a long asymptomatic period. However, late-stage atherosclerosis is progressive and irreversible, leading to nearly 50% of Western society's deaths (Lusis, 2000). Atherosclerosis begins in adolescence as deposits of cholesterol and its esters in the intima of large muscular arteries and remains asymptomatic over decades. At advanced stages, growing atherosclerotic plaques or plaque rupture-induced thrombi can obstruct blood flow and can lead to detrimental consequences such as heart attack, stroke, and peripheral vascular disease (Libby, 2021; Brassington et al., 2022). Therapeutic strategies for reversing or retarding the progression of atherosclerotic plaque or lesion formation rarely achieve a satisfactory outcome. Therefore, early detection of atherosclerosis is important for CVD prevention and treatment.

Early detection of atherosclerosis has been always a great challenge for the healthcare system, however. Angiography is the gold standard for diagnosis of atherosclerotic vascular disease, but this invasive method is expensive and inconvenient and is often accompanied by complications such as allergic reactions, kidney damage, heart attack, or stroke, thereby rendering it unsuitable for regular check-ups (Tavakol et al., 2012).

Patient sample biopsies present a convenient, economical, and minimally invasive alternative for diagnosis and monitoring the progress of atherosclerosis (Stastna & Eyk, 2012). However, traditional atherosclerosis-associated biomarkers in patient samples, such as cholesterol, LDL, and triglycerides, do not provide acceptable diagnostic accuracy in patients with low to moderate cardiovascular risk. For example, several biomarkers, such as inflammatory biomarkers (interleukin 6, tumor necrosis factor-a, and C-reactive protein), ox-LDL, and circulating microRNAs, have been proposed for diagnosing atherosclerosis (Surma et al., 2020). However, these biomarkers are either non-specific or are insensitive to early-stage atherosclerosis.

Thus, there is a need for a non-invasive diagnostic that can rapidly and reliably identify biomarkers for early diagnosis of atherosclerosis.

### SUMMARY

The present disclosure fulfills the aforementioned need by providing sensitive diagnostic methods and kits that do not require invasive angiography. It was found that the Yes-associated protein (YAP)-induced secretory protein TNFAIP2 is highly expressed in the vascular endothelia surrounding atherosclerotic plaques and in the sera of subjects with early stage of atherosclerosis, and severity of atherosclerosis is correlated with serum levels of TNFAIP2. This novel biomarker can be used to accurately and efficiently diagnose, prognose, and/or monitor a subject having, suspected of having, or at risk of having atherosclerosis. Accordingly, aspects of the disclosure relate to a method of measuring, detecting, or assaying a TNFAIP2 level comprising: obtaining a biological sample from a subject; and measuring the TNFAIP2 level in a biological sample from the subject as compared to a control. Also disclosed is a method for treating or preventing atherosclerosis in a subject, the method comprising administering an effective amount of a treatment for atherosclerosis to the subject after measuring, detecting, or assaying the TNFAIP2 level in a biological sample from the subject as compared to a control.

In some aspects, the control comprises a level of TNFAIP2 that is representative of a level of TNFAIP2 in a biological sample from a subject without atherosclerosis. In some aspects, TNFAIP2 is not detected in the sample. In some aspects, the measured, detected, or assayed TNFAIP2 level is not significantly different from the control. In some aspects, the measured, detected, or assayed TNFAIP2 level is less than the control. In some aspects, a treatment for atherosclerosis is not administered to the subject. In some aspects, the measured, detected, or assayed TNFAIP2 level is greater than the control. In some aspects, the method further comprises administering a treatment for atherosclerosis to the subject.

In some aspects, the control comprises a level of TNFAIP2 that is representative of a level of TNFAIP2 in a biological sample from a subject with atherosclerosis. In some aspects, TNFAIP2 is not detected in the sample. In some aspects, the measured, detected, or assayed TNFAIP2 level is not significantly different from the control. In some aspects, the measured, detected, or assayed TNFAPI2 level is less than the control. In some aspects, a treatment for atherosclerosis is not administered to the subject. In some aspects, the measured, detected, or assayed TNFAIP2 level is not significantly different from the control. In some aspects, the measured, detected, or assayed TNFAIP2 level is greater than the control. In some aspects, the method further comprises administering a treatment for atherosclerosis to the subject.

Further aspects of the disclosure relate to:

A method of measuring, detecting, or assaying a TNFAIP2 level comprising: obtaining a biological sample from a subject having, suspected of having, or at risk of having atherosclerosis; and measuring, detecting, or assaying the TNFAIP2 level in a biological sample from the subject as compared to a control.

A method for evaluating a subject comprising measuring, detecting, or assaying TNFAIP2 in a biological sample from the subject.

A method for treating or preventing atherosclerosis in a subject, the method comprising administering a treatment for the atherosclerosis to the subject after the level of TNFAIP2 has been measured, detected, or assayed in a biological sample from the subject.

A method for treating or preventing atherosclerosis in a subject, the method comprising administering a treatment for the atherosclerosis to a subject having a level of TNFAIP2 in a biological sample that is higher than a control.

A method of diagnosing a subject as being at risk of having atherosclerosis, comprising: a) measuring, detecting, or assaying TNFAIP2 in a biological sample from the subject; b) comparing the measured, detected, or assayed level to control level or control samples; and c) diagnosing the subject being at risk of having atherosclerosis based on the measured, detected, or assayed level of TNFAIP2.

A method of diagnosing or prognosing a subject with atherosclerosis, comprising: a) measuring, detecting, or assaying TNFAIP2 in a biological sample from the subject; b) comparing the measured, detected, or assayed level to control level or control samples; and c) diagnosing or prognosing the subject with atherosclerosis based on the measured, detected, or assayed level of TNFAIP2.

A method of monitoring progression and/or severity of atherosclerosis in a subject, comprising: a) measuring, detecting, or assaying TNFAIP2 in a biological sample from the subject; b) comparing the measured, detected, or assayed level to control level or control samples; and c) determining the progression and/or severity of atherosclerosis in the subject based on the measured, detected, or assayed level of TNFAIP2.

A method of monitoring a subject being treated for atherosclerosis with a therapeutic agent, comprising: a) measuring, detecting, or assaying TNFAIP2 in a biological sample from the subject; b) comparing the measured, detected, or assayed level to control level or control samples; and c) determining the efficacy of the therapeutic agent based on the measured, detected, or assayed level of TNFAIP2.

Further aspects relate to a kit comprising one or more detection agents for measuring, detecting, or assaying an expression level of TNFAIP2 in a sample from a subject. In some aspects, the kit may be used for: diagnosing a subject as being at risk of having atherosclerosis; diagnosing a subject as having atherosclerosis; prognosing a subject with atherosclerosis; monitoring progression and/or severity of atherosclerosis in a subject; and/or monitoring a subject being treated for atherosclerosis with a therapeutic agent.

Further aspects relate to a method for making a complex comprising contacting a biological sample with an antibody that binds to TNFAIP2 or a TNFAIP2 binding fragment thereof.

Further aspects of the disclosure relate to:

A method of measuring, detecting, or assaying a TNFAIP2 level comprising: obtaining a biological sample from a subject having, suspected of having, or at risk of having atherosclerosis; and measuring, detecting, or assaying the TNFAIP2 level in a biological sample from the subject as compared to a control using a detection agent comprising an anti-TNFAIP2 antibody or a TNFAIP2 binding fragment thereof.

A method for evaluating a subject comprising measuring, detecting, or assaying TNFAIP2 in a biological sample from the subject using a detection agent comprising an anti-TNFAIP2 antibody or a TNFAIP2 binding fragment thereof.

A method for treating or preventing atherosclerosis in a subject, the method comprising administering a treatment for the atherosclerosis to the subject after the level of TNFAIP2 has been measured, detected, or assayed in a biological sample from the subject using a detection agent comprising an anti-TNFAIP2 antibody or a TNFAIP2 binding fragment thereof.

A method for treating or preventing atherosclerosis in a subject, the method comprising administering a treatment for the atherosclerosis to a subject having a level of TNFAIP2 in a biological sample that is higher than a control based on measured, detected, or assayed TNFAIP2 levels in a biological sample from the subject using a detection agent comprising an anti-TNFAIP2 antibody or a TNFAIP2 binding fragment thereof.

A method of diagnosing a subject as being at risk of having atherosclerosis, comprising: a) measuring, detecting, or assaying TNFAIP2 in a biological sample from the subject using a detection agent comprising an anti-TNFAIP2 antibody or a TNFAIP2 binding fragment thereof; b) comparing the measured, detected, or assayed level to control level or control samples; and c) diagnosing the subject being at risk of having atherosclerosis based on the measured, detected, or assayed level of TNFAIP2.

A method of diagnosing or prognosing a subject with atherosclerosis, comprising: a) measuring, detecting, or assaying TNFAIP2 in a biological sample from the subject using a detection agent comprising an anti-TNFAIP2 antibody or a TNFAIP2 binding fragment thereof; b) comparing the measured, detected, or assayed level to control level or control samples; and c) diagnosing or prognosing the subject with atherosclerosis based on the measured, detected, or assayed level of TNFAIP2.

A method of monitoring progression and/or severity of atherosclerosis in a subject, comprising: a) measuring, detecting, or assaying TNFAIP2 in a biological sample from the subject using a detection agent comprising an anti-TNFAIP2 antibody or a TNFAIP2 binding fragment thereof; b) comparing the measured, detected, or assayed level to control level or control samples; and c) determining the progression and/or severity of atherosclerosis in the subject based on the measured, detected, or assayed level of TNFAIP2.

A method of monitoring a subject being treated for atherosclerosis with a therapeutic agent, comprising: a) measuring, detecting, or assaying TNFAIP2 in a biological sample from the subject using a detection agent comprising an anti-TNFAIP2 antibody or a TNFAIP2 binding fragment thereof; b) comparing the measured, detected, or assayed level to control level or control samples; and c) determining the efficacy of the therapeutic agent based on the measured, detected, or assayed level of TNFAIP2.

Methods of the disclosure include prognosing, diagnosing, monitoring, and/or treating a subject having atherosclerosis, suspected of having atherosclerosis, at risk of having, and/or having symptoms of atherosclerosis. The subject may be diagnosed as being at risk of having or having atherosclerosis based on the evaluated level of monomeric TNFAIP2.

The term atherosclerosis refers to a vascular disease involving the gradual buildup of, e.g., fats, cholesterol, calcium, and other substances in and on artery walls. This buildup may be called a plaque or lesion. The plaque can cause arteries to become thick, stiff, or hardened and narrow, thereby blocking blood flow to organs and tissues. The plaque can also burst, leading to a blood clot. Risk factors include, e.g., high cholesterol, high blood pressure, diabetes, tobacco use, obesity, lack of exercise, and a diet high in saturated fat.

The biological sample may comprise serum, plasma, or tissue samples. In some aspects, the biological sample is a blood sample or a fraction thereof. In some aspects, the biological sample is a biological sample described herein. In certain aspects, the biological sample is a serum sample. In some aspects, the biological sample is a plasma sample.

In aspects of the disclosure, measuring, detecting, or assaying TNFAIP2 in a biological sample from the subject comprises immunological detection of TNFAIP2 in the biological sample. In some aspects, measuring, detecting, or assaying TNFAIP2 comprises an enzyme-linked immunosorbent assay (ELISA) assay. In some aspects, an ELISA can utilize an anti-TNFAIP2 antibody or TNFAIP2 binding molecule comprising: an antibody having a V_{L} chain having an amino acid sequence with at least 85% identity to SEQ ID NO:1 and a V_{H} chain having an amino acid sequence with at least 85% identity to SEQ ID NO:2; and/or an antibody having a V_{L} chain having an amino acid sequence with at least 85% identity to SEQ ID NO: 3 and a V_{H} chain having an amino acid sequence with at least 85% identity to SEQ ID NO: 4. An ELISA assay uses a solid-phase type of enzyme immunoassay (EIA) to detect the presence of a ligand (commonly a protein) in a liquid sample using antibodies directed against the protein to be measured. In some aspects, antigens from the biological sample or fraction thereof are attached to a surface. Then, an antibody, such as an anti-TNFAIP2 antibody, may be applied over the surface so it can bind to any TNFAIP2 from the biological sample. This antibody may be linked to a detection molecule, such as an enzyme, and then any unbound antibodies may be removed. In the final step, the detection molecule may be detected qualitatively or quantitatively. In aspects in which the detection molecule is an enzyme, the enzyme's substrate may be added, leading to a reaction that produces a detectable signal, e.g., a color change that can be quantitatively or qualitatively measured.

In some aspects, the ELISA is further characterized as a sandwich ELISA. An anti-TNFAIP2 antibody may be immobilized on a solid support, such as a microtiter plate or a polystyrene microtiter plate. The biological sample or fraction may be added to the solid support to allow binding between the anti-TNFAIP2 antibody and TNFAIP2 in the biological sample or fraction. Unbound molecules may be washed away from the solid support. After the TNFAIP2 antigen is immobilized, the detection antibody can be added, forming a complex with the antigen. The detection antibody can be covalently linked to a detection molecule, such as an enzyme or can itself be detected by a secondary antibody that is linked to a detection molecule, such as an enzyme. Between each step, the plate is typically washed with a mild detergent solution to remove any proteins or antibodies that are non-specifically bound. After the final wash step, the plate is developed by qualitatively or quantitatively detecting the detection molecule. In the case of enzymatic detection, the final step may comprise adding an enzymatic substrate to produce a visible signal that can qualitatively or quantitatively detected.

The ELISA may be performed using other forms of ligand binding assays instead of strictly immunoassays. An ELISA may be one that comprises any ligating reagent that can be immobilized on the solid phase along with a detection reagent that will bind specifically and use a detectable molecule to generate a signal that can be properly quantified. In between the washes, only the ligand and its specific binding counterparts remain specifically bound or “immunosorbed” by antigen-antibody interactions to the solid phase, while the nonspecific or unbound components are washed away.

“Detectable labels, molecules, or moieties” or “detection molecules, labels, or moieties” are used interchangeably and refer to compounds and/or elements that can be detected due to their specific functional properties, and/or chemical characteristics, the use of which allows the antibody to be detected, and/or further quantified if desired. Examples of detectable labels include, but not limited to, radioactive isotopes, fluorescers, semiconductor nanocrystals, chemiluminescers, chromophores, enzymes, enzyme substrates, enzyme cofactors, enzyme inhibitors, dyes, metal ions, metal sols, ligands (e.g., biotin, streptavidin or haptens) and the like. Particular examples of labels are, but not limited to, horseradish peroxidase (HRP), fluorescein, FITC, rhodamine, dansyl, umbelliferone, dimethyl acridinium ester (DMAE), Texas red, luminol, NADPH and α- or β-galactosidase. Antibody conjugates include those intended primarily for use in vitro, where the antibody is linked to a secondary binding ligand and/or to an enzyme to generate a colored product upon contact with a chromogenic substrate. Examples of suitable enzymes include, but are not limited to, urease, alkaline phosphatase, (horseradish) hydrogen peroxidase, or glucose oxidase. Preferred secondary binding ligands are biotin and/or avidin and streptavidin compounds. The uses of such labels is well known to those of skill in the art and are described, for example, in U.S. Pat. Nos. 3,817,837; 3,850,752; 3,939,350; 3,996,345; 4,277,437; 4,275,149; and 4,366,241; each incorporated herein by reference. Molecules containing azido groups may also be used to form covalent bonds to proteins through reactive nitrene intermediates that are generated by low intensity ultraviolet light (Potter & Haley, 1983).

In some aspects, measuring, detecting, or assaying TNFAIP2 comprises contacting the biological sample with an anti-TNFAIP2 antibody or TNFAIP2 binding molecule under conditions that allow for the binding of TNFAIP2 to the anti-TNFAIP2 antibody. In some aspects, the anti-TNFAIP2 antibody or TNFAIP2 binding molecule comprises: an antibody having a V_{L} chain having an amino acid sequence with at least 85% identity to SEQ ID NO: 1 and a V_{H} chain having an amino acid sequence with at least 85% identity to SEQ ID NO:2; and/or an antibody having a V_{L} chain having an amino acid sequence with at least 85% identity to SEQ ID NO:3 and a V_{H} chain having an amino acid sequence with at least 85% identity to SEQ ID NO:4. In some aspects, the anti-TNFAIP2 antibody or binding molecule is linked to a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The methods of the disclosure may comprise or further comprise washing the solid support to remove unbound molecules. In some aspects, the methods comprise or further comprise contacting the biological sample or the fraction with a capture antibody or antigen-binding molecule. In some aspects, the capture antibody or antigen-binding molecule comprise a second anti-TNFAIP2 antibody or TNFAIP2 antigen-binding fragment. Exemplary antigen-binding fragments include, for example, a single chain variable fragment (scFv), F(ab′)₂, Fab′, Fab, Fv, or rIgG. In some aspects, the capture antibody is linked to a detectable label. The method may comprise or further comprise quantitatively or qualitatively measuring, detecting, or assaying the detectable label.

Aspects include compositions comprising one or more TNFAIP2-binding proteins. Aspects include a TNFAIP2-binding protein comprising one or more regions (e.g., heavy chain variable region, light chain variable region, etc.). Aspects include monoclonal antibodies, chimeric antibodies, humanized antibodies, and antibody-like molecules. Aspects also include nucleic acid molecules encoding for one or more antigen-binding proteins or portions thereof. Aspects include recombinant, transformed, or modified cells, vectors, and/or expression cassettes comprising such nucleic acid molecules. In some aspects, the compositions contemplated herein can comprise 1, 2, 3, 4, 5, or more of the following components: an antigen-binding protein, a nucleic acid, a vector, a cell, a polypeptide, an oligonucleotide, a complementarity determining region, a light chain variable region, a heavy chain variable region, a light chain constant region, a heavy chain constant region, and complementarity determining regions. Any one or more of these components may be excluded from the disclosed compositions.

In some aspects, the disclosed antigen-binding proteins, antibodies, antibody-like molecules, and fragments thereof are TNFAIP2-binding proteins (i.e., are capable of binding to TNFAIP2). In some aspects, the TNFAIP2-binding proteins are monoclonal anti-TNFAIP2 antibodies. In some aspects, the TNFAIP2-binding proteins are chimeric anti-TNFAIP2 antibodies. In some aspects, the TNFAIP2-binding proteins are humanized anti-TNFAIP2 antibodies. Antigen-binding proteins described herein may be used in treating one or more conditions associated with expression or activity of TNFAIP2 such as, for example, atherosclerosis. In some aspects, TNFAIP2-binding proteins are used in treating one or more TNFAIP2-associated conditions with reduced risk of toxicity and improved efficacy as compared to previously disclosed treatments for a condition (e.g., atherosclerosis).

Some aspects are directed to a nucleic acid encoding for a polypeptide or fragment thereof that specifically binds TNFAIP2. In some aspects, the nucleic acid encodes both the heavy chain variable region and the light chain variable region of the TNFAIP2-binding protein. In some aspects, the nucleic acid encodes the light chain variable region of the TNFAIP2-binding protein. In some aspects, the nucleic acid encodes the heavy chain variable region of the TNFAIP2-binding protein. Also disclosed is a vector comprising one or more of the nucleic acids disclosed herein. In some aspects, the nucleic acid(s) comprised in the vector is operably linked to an expression control sequence.

In some aspects, an anti-TNFAIP2 antibody or a TNFAIP2 binding fragment thereof comprises a light chain variable region (V_{L}) having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:1 or 3, and a heavy chain variable region (V_{H}) having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:2 or 4. In some aspects, the V_{L} has at least 85% identity to SEQ ID NO: 1 or 3. In some aspects, the V_{H} has at least 85% identity to SEQ ID NO:2 or 4. In some aspects, the V_{L} has at least 90% identity to SEQ ID NO:1 or 3. In some aspects, the V_{H} has at least 90% identity to SEQ ID NO:2 or 4. In some aspects, the V_{L} has at least 95% identity to SEQ ID NO: 1 or 3. In some aspects, the V_{H} has at least 95% identity to SEQ ID NO:2 or 4. In some aspects, the V_{L} comprises SEQ ID NO: 1 or 3. In some aspects, the VII comprises SEQ ID NO: 2 or 4.

In some aspects, the V_{L} has at least 85% identity to SEQ ID NO: 1 and the V_{H} has at least 85% identity to SEQ ID NO:2. In some aspects, the V_{L} has at least 90% identity to SEQ ID NO: 1 and the V_{H} has at least 90% identity to SEQ ID NO:2. In some aspects, the V_{L} has at least 95% identity to SEQ ID NO: 1 and the V_{H} has at least 95% identity to SEQ ID NO:2. In some aspects, the V_{L} comprises SEQ ID NO: 1 and the V_{H} comprises SEQ ID NO:2.

In some aspects, the V_{L} has at least 85% identity to SEQ ID NO:3 and the V_{H} has at least 85% identity to SEQ ID NO:4. In some aspects, the V_{L} has at least 90% identity to SEQ ID NO: 3 and the V_{H} has at least 90% identity to SEQ ID NO:4. In some aspects, the V_{L} has at least 95% identity to SEQ ID NO:3 and the V_{H} has at least 95% identity to SEQ ID NO:4. In some aspects, the V_{L} comprises SEQ ID NO:3 and the V_{H} comprises SEQ ID NO:4.

In some aspects, the disclosure relates to an anti-TNFAIP2 antibody or a TNFAIP2 binding fragment thereof comprising: a light chain variable region (V_{L}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:9, SEQ ID NO: 10, SEQ ID NO:11, SEQ ID NO: 15, SEQ ID NO: 16, or SEQ ID NO:17; and a heavy chain variable region (V_{H}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO: 12, SEQ ID NO: 13, SEQ ID NO:14. SEQ ID NO:18, SEQ ID NO: 19, or SEQ ID NO:20.

In some aspects, the disclosure relates to an anti-TNFAIP2 antibody or a TNFAIP2 binding fragment thereof comprising: a light chain variable region (V_{L}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:9, SEQ ID NO: 10, and SEQ ID NO:11; and a heavy chain variable region (V_{H}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:12, SEQ ID NO: 13, and SEQ ID NO: 14.

In some aspects, the disclosure relates to an anti-TNFAIP2 antibody or a TNFAIP2 binding fragment thereof comprising: a light chain variable region (V_{L}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:15, SEQ ID NO: 16, and SEQ ID NO:17; and a heavy chain variable region (V_{H}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:18, SEQ ID NO: 19, and SEQ ID NO:20.

In some aspects, the TNFAIP2-binding protein has an affinity for TNFAIP2. In some aspects, the TNFAIP2-binding protein has an affinity for TNFAIP2 of between 0.001 and 1000 nM. In some aspects, the TNFAIP2-binding protein has an affinity for TNFAIP2 of between 0.01 and 100 nM. In some aspects, the TNFAIP2-binding protein has an association constant for a TNFAIP2 protein of between 0.1 and 50 nM. In some aspects, the TNFAIP2-binding protein has an association constant for a TNFAIP2 protein of between 1 and 20 nM. In some aspects, the TNFAIP2-binding protein has an association constant for a TNFAIP2 protein of at least, at most, exactly, or between (inclusive or exclusive) any two of 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21, 21.5, 22, 22.5, 23, 23.5, 24, 24.5, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, or 1000 nM, or any range derivable therein. In some aspects, the TNFAIP2-binding protein has an affinity for TNFAIP2 of about 10 nM.

A further aspect is directed to a polypeptide that specifically binds TNFAIP2 prepared by a method described herein. Some aspects are directed to a method for generating a TNFAIP2-binding protein, such as a TNFAIP2-binding protein described herein, by culturing a cell described herein under conditions sufficient to express a TNFAIP2-binding protein disclosed herein in the cell. Some aspects are directed to a method for generating a TNFAIP2-binding protein, such as a TNFAIP2-binding protein described herein, comprising (a) providing to a cell a nucleic acid encoding for the TNFAIP2-binding protein; and (b) subjecting the cell to conditions sufficient to express the nucleic acid in the cell.

The methods may comprise or further comprise quantitating the measured, detected, or assayed level of TNFAIP2 in the biological sample. In some aspects, the level of TNFAIP2 is normalized. In some aspects, the level of TNFAIP2 is compared to a control. In some aspects, the measured, detected, or assayed level of TNFAIP2 is determined to be greater than the control. In some aspects, the measured, detected, or assayed level of TNFAIP2 is determined to be less than the control. In some aspects, the subject has or has been determined to have a measure, detected, or assayed level of TNFAIP2 in the biological sample that is greater than a level of TNFAIP2 in a control sample. In some aspects, the subject has or has been determined to have a measure, detected, or assayed level of TNFAIP2 in the biological sample that is less than a level of TNFAIP2 in a control sample. In some aspects, the subject has or has been determined to have a measure, detected, or assayed level of TNFAIP2 in the biological sample that is not significantly different than a level of TNFAIP2 in a control sample.

For example, the measure, detected, or assayed level of TNFAIP2 may be determined to be, to be at least, to be at most, or to be between (inclusive or exclusive) any two of 0.5, 1, 1.5, 2, 2.5, 3, 3.5, 4, or 5 standard deviations different than or within a control value. In some aspects, the measure, detected, or assayed level of TNFAIP2 may be determined to be, to be at least, to be at most, or to be between (inclusive or exclusive) any two of 1, 2, 5, 10, 15, 20, 25, 30, 35, 40, 45, 50, 55, 60, 65, 70, 75, 80, 85, 90, 95, 98, 99, or 100% (or any derivable range therein) above or below a control level of TNFAIP2.

The control may comprise a level of TNFAIP2 that is representative of a level of TNFAIP2 in a biological sample from a subject with or without atherosclerosis. In some instances, the control may comprise a level of TNFAIP2 that is representative of a level of TNFAIP2 in a biological sample from a subject with atherosclerosis. In some aspects, the control may comprise a level of TNFAIP2 that is representative of a level of TNFAIP2 in a biological sample from a subject without atherosclerosis.

In some aspects, the method comprises or further comprises diagnosing the subject as being at risk of having atherosclerosis. In some aspects, the subject is diagnosed as being at risk of having atherosclerosis based on the measure, detected, or assayed level of TNFAIP2. The methods of the disclosure may comprise or further comprise treating or preventing atherosclerosis in the subject. In some aspects, a method of diagnosing a subject as being at risk of having atherosclerosis comprises: a) measuring, detecting, or assaying a level of TNFAIP2 in a biological sample from the subject; b) comparing the measured, detected, or assayed level of TNFAIP2 to control level or control samples; and c) diagnosing the subject being at risk of having atherosclerosis based on the measured, detected, or assayed level of TNFAIP2. In some aspects, the subject is diagnosed with a low risk of having atherosclerosis when the measured, detected, or assayed level of TNFAIP2: i) is not measured, detected, or assayed in the biological sample from the subject; ii) is not significantly different than a control, wherein the control comprises a level of TNFAIP2 that is representative of a level of TNFAIP2 in a biological sample from a subject without atherosclerosis; or iii) is less than a control, wherein the control comprises a level of TNFAIP2 that is representative of a level of TNFAIP2 in a biological sample from a subject with atherosclerosis. In some aspects, the subject is diagnosed with a high risk of having atherosclerosis when the measured, detected, or assayed level of TNFAIP2: i) is greater than a control, wherein the control comprises a level of TNFAIP2 that is representative of the level of TNFAIP2 in a biological sample from a subject without atherosclerosis; or ii) is not significantly different than a control; wherein the control comprises a level of TNFAIP2 that is representative of the level of TNFAIP2 in a biological sample from a subject with atherosclerosis. In some aspects, the subject is diagnosed with a low risk of having atherosclerosis when TNFAIP2 is not measured, detected, or assayed in the biological sample from the subject. In some aspects, the subject is diagnosed with a high risk of having atherosclerosis when TNFAIP2 is measured, detected, or assayed in the biological sample from the subject. In some aspects, a low risk is indicative of a subject with a low risk for atherosclerotic plaque formation. In some aspects, a high risk is indicative of a subject with a high risk for atherosclerotic plaque formation. In some aspects, the method further comprises administering an effective amount of a treatment for atherosclerosis to the subject diagnosed with a low risk of having atherosclerosis. In some aspects, the method further comprises administering an effective amount of a treatment for atherosclerosis to the subject diagnosed with a high risk of having atherosclerosis.

In some aspects, the method comprises or further comprises diagnosing or prognosing the subject with atherosclerosis. In some aspects, the subject is diagnosed with atherosclerosis based on the measured, detected, or assayed level of TNFAIP2. The methods of the disclosure may comprise or further comprise treating the subject for atherosclerosis. In some aspects, a method of diagnosing a subject comprises: a) measuring, detecting, or assaying a level of TNFAIP2 in a biological sample from the subject; b) comparing the measured, detected, or assayed level of TNFAIP2 to control level or control samples; and c) diagnosing or prognosing the subject with atherosclerosis based on the measured, detected, or assayed level of TNFAIP2. In some aspects, the subject is diagnosed as not having atherosclerosis when the measured, detected, or assayed level of TNFAIP2: i) is not measured, detected, or assayed in the biological sample from the subject; ii) is not significantly different than a control, wherein the control comprises the level of TNFAIP2 that is representative of a level of TNFAIP2 in a biological sample from a subject without atherosclerosis; or iii) is less than a control, wherein the control comprises a level of TNFAIP2 that is representative of a level of TNFAIP2 in a biological sample from a subject with atherosclerosis. In some aspects, the subject is diagnosed as having atherosclerosis when the measured, detected, or assayed level of TNFAIP2: i) is greater than a control, wherein the control comprises a level of TNFAIP2 that is representative of the level of TNFAIP2 in a biological sample from a subject without atherosclerosis; or ii) is not significantly different than a control; wherein the control comprises a level of TNFAIP2 that is representative of the level of TNFAIP2 in a biological sample from a subject with atherosclerosis. In some aspects, the subject is diagnosed as not having atherosclerosis when TNFAIP2 is not measured, detected, or assayed in the biological sample from the subject. In some aspects, the subject is diagnosed as having atherosclerosis when TNFAIP2 is measured, detected, or assayed in the biological sample from the subject. In some aspects, the method further comprises administering an effective amount of a treatment for atherosclerosis to a subject diagnosed with atherosclerosis.

In some aspects, the subject is prognosed as low risk when the measured, detected, or assayed level of TNFAIP2: i) is not measured, detected, or assayed in the biological sample from the subject; ii) is not significantly different than a control, wherein the control comprises the level of TNFAIP2 that is representative of a level of TNFAIP2 in a biological sample from a subject without atherosclerosis; or iii) is less than a control, wherein the control comprises a level of TNFAIP2 that is representative of a level of TNFAIP2 in a biological sample from a subject with atherosclerosis. In some aspects, low risk is indicative of a subject with a low risk for atherosclerotic plaque formation. In some aspects, the subject is prognosed as high risk when the measured, detected, or assayed level of TNFAIP2: i) is greater than a control, wherein the control comprises a level of TNFAIP2 that is representative of the level of TNFAIP2 in a biological sample from a subject without atherosclerosis; or ii) is not significantly different than a control; wherein the control comprises a level of TNFAIP2 that is representative of the level of TNFAIP2 in a biological sample from a subject with atherosclerosis. In some aspects, high risk is indicative of a subject with a high risk for atherosclerotic plaque formation. In some aspects, the subject is prognosed as low risk as when TNFAIP2 is not measured, detected, or assayed in the biological sample from the subject. In some aspects, the subject is prognosed as high risk when TNFAIP2 is measured, detected, or assayed in the biological sample from the subject. In some aspects, the method further comprises administering an effective amount of a treatment for atherosclerosis to a subject prognosed as low risk. In some aspects, the method further comprises administering an effective amount of a treatment for atherosclerosis to a subject prognosed as high risk.

In some aspects, the method comprises or further comprises monitoring the progression and/or severity of atherosclerosis in the subject. In some aspects, atherosclerosis progression and/or severity is determined based on the measured, detected, or assayed level of TNFAIP2. The methods of the disclosure may comprise or further comprise treating the subject for atherosclerosis. In some aspects, a method of monitoring progression and/or severity of atherosclerosis in the subject comprises: a) measuring, detecting, or assaying a level of TNFAIP2 in a biological sample from the subject; b) comparing the measured, detected, or assayed level of TNFAIP2 to control level or control samples; and c) determining the progression and/or severity of atherosclerosis in the subject based on the measured, detected, or assayed level of TNFAIP2. In some aspects, atherosclerosis is considered earlier stage and/or less severe when the measured, detected, or assayed level of TNFAIP2: i) is not measured, detected, or assayed in the biological sample from the subject; ii) is not significantly different than a control, wherein the control comprises a level of TNFAIP2 that is representative of a level of TNFAIP2 in a biological sample from a subject without atherosclerosis; iii) is less than a control, wherein the control comprises a level of TNFAIP2 that is representative of a level of TNFAIP2 in a biological sample from a subject with atherosclerosis; or iv) is decreased compared to a level of TNFAIP2 before treatment of the subject with the therapeutic agent. In some aspects, atherosclerosis is considered later stage and/or more severe when the measured, detected, or assayed level of TNFAIP2: i) is measured, detected, or assayed in the biological sample from the subject; ii) is increased compared to a control, wherein the control comprises a level of TNFAIP2 that is representative of a level of TNFAIP2 in a biological sample from a subject without atherosclerosis; iii) is not significantly different or more than a control, wherein the control comprises a level of TNFAIP2 that is representative of a level of TNFAIP2 in a biological sample from a subject with atherosclerosis; or iv) is not significantly different or is increased compared to a level of TNFAIP2 before treatment of the subject with the therapeutic agent. In some aspects, atherosclerotic plaque formation is indicative of later stage and/or more severe atherosclerosis in the subject. In some aspects, the method further comprises measuring, detecting, or assaying the level of TNFAIP2 in a biological sample previously obtained from the subject. In some aspects, the method further comprises measuring, detecting, or assaying the level of TNFAIP2 in a biological sample from the subject obtained after diagnosis with atherosclerosis. For example, the method may comprise or further comprise measuring, detecting, or assaying TNFAIP2 levels one, two, three, four, five, six, or more weeks, months, or years after diagnosis with atherosclerosis. Atherosclerosis progression and/or severity may be determined based on the measured, detected, or assayed levels of TNFAIP2. In some aspects, the method further comprises administering an effective amount of a treatment for atherosclerosis to the subject having early-stage and/or less severe atherosclerosis. In some aspects, the method further comprises administering an effective amount of a treatment for atherosclerosis to the subject having later-stage and/or more severe atherosclerosis.

In some aspects, the method comprises or further comprises monitoring a subject being treated for atherosclerosis with a therapeutic agent. In some aspects, efficacy of the therapeutic agent is determined based on the determined level of TNFAIP2. In some aspects, a method of monitoring a subject being treated for atherosclerosis with a therapeutic agent comprises: a) measuring, detecting, or assaying a level of TNFAIP2 in a biological sample from the subject; b) comparing the measured, detected, or assayed level of TNFAIP2 to control level or control samples; and c) determining the efficacy of the therapeutic agent based on the measured, detected, or assayed level of TNFAIP2. In some aspects, the therapeutic agent is determined to be effective when the measured, detected, or assayed level of TNFAIP2: i) is not measured, detected, or assayed in the biological sample from the subject; ii) is not significantly different than a control, wherein the control comprises a level of TNFAIP2 that is representative of a level of TNFAIP2 in a biological sample from a subject without atherosclerosis; iii) is less than a control, wherein the control comprises a level of TNFAIP2 that is representative of a level of TNFAIP2 in a biological sample from a subject with atherosclerosis; or iv) is decreased compared to a level of TNFAIP2 before treatment of the subject with the therapeutic agent. In some aspects, the therapeutic agent is determined to be ineffective when the measured, detected, or assayed level of TNFAIP2: i) measured, detected, or assayed in the biological sample from the subject; ii) is increased compared to a control, wherein the control comprises a level of TNFAIP2 that is representative of a level of TNFAIP2 in a biological sample from a subject without atherosclerosis; iii) is not significantly different or more than a control, wherein the control comprises a level of TNFAIP2 that is representative of a level of TNFAIP2 in a biological sample from a subject with atherosclerosis; or iv) is not significantly different or is increased compared to a level of TNFAIP2 before treatment of the subject with the therapeutic agent. In some aspects, the method further comprises measuring, detecting, or assaying the level of TNFAIP2 in a biological sample from the subject obtained prior to treatment. In some aspects, the method further comprises measuring, detecting, or assaying the level of TNFAIP2 in a biological sample from the subject obtained after one or more treatments. For example, the method may comprise or further comprise measuring, detecting, or assaying TNFAIP2 levels after one dose, after two doses, after three doses, after four doses, after five doses, and/or after six doses of a particular treatment. Treatment efficacy may be determined based on the measured, detected, or assayed levels of TNFAIP2.

In some aspects, the measured, detected, or assayed level of TNFAIP2 is determined to be, determined to be at least, determined to be at most, or determined to be between (inclusive or exclusive) any two of 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 325, 350, 375, 400, 425, 450, 475, or 500 ng/ml, mcg/ml, mg/ml, (or any derivable range therein) in the biological sample from the subject. In some aspects, the subject is diagnosed as being at risk of having atherosclerosis when the measured, detected, or assayed level of TNFAIP2 is determined to be, determined to be at least, determined to be at most, or determined to be between (inclusive or exclusive) any two of 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 325, 350, 375, 400, 425, 450, 475, or 500 ng/ml, mcg/ml, mg/ml, (or any derivable range therein) in the biological sample from the subject. In some aspects, the subject is diagnosed with atherosclerosis when the measured, detected, or assayed level of TNFAIP2 is determined to be, determined to be at least, determined to be at most, or determined to be between (inclusive or exclusive) any two of 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 325, 350, 375, 400, 425, 450, 475, or 500 ng/ml, mcg/ml, mg/ml, (or any derivable range therein) in the biological sample from the subject. In some aspects, the subject is prognosed with high risk for atherosclerotic plaque formation when the measured, detected, or assayed level of TNFAIP2 is determined to be, determined to be at least, determined to be at most, or determined to be between (inclusive or exclusive) any two of 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 325, 350, 375, 400, 425, 450, 475, or 500 ng/ml, mcg/ml, mg/ml, (or any derivable range therein) in the biological sample from the subject. In some aspects, the subject is diagnosed with severe or later stage atherosclerosis when the measured, detected, or assayed level of TNFAIP2 is determined to be, determined to be at least, determined to be at most, or determined to be between (inclusive or exclusive) any two of 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 325, 350, 375, 400, 425, 450, 475, or 500 ng/ml, mcg/ml, mg/ml, (or any derivable range therein) in the biological sample from the subject. In some aspects, a therapeutic agent is determined to be ineffective when the measured, detected, or assayed level of TNFAIP2 is determined to be, determined to be at least, determined to be at most, or determined to be between (inclusive or exclusive) any two of 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 325, 350, 375, 400, 425, 450, 475, or 500 ng/ml, mcg/ml, mg/ml, (or any derivable range therein) in the biological sample from the subject.

In aspects of the disclosure, the subject or patient may be a human subject or a non-human subject. In some aspects, the subject or patient is a non-human animal. The subject may further be defined as a high-risk subject. The subject may be one that has been diagnosed as being at risk of having atherosclerosis. The subject may be one having one or more symptoms of atherosclerosis. The subject may also be one that has been diagnosed with atherosclerosis. In some aspects, the subject has been treated for atherosclerosis. In some aspects, the subject will be treated for atherosclerosis. In some aspects, the subject is currently undergoing treatment for atherosclerosis.

Atherosclerosis-related cardiovascular diseases can include, e.g., carotid artery disease, coronary artery disease, heart attack, mesenteric ischemia, peripheral artery disease, renal artery stenosis, stroke, transient ischemic attack (TIA), or a combination thereof. In some aspects, the subject is one that has one or more symptoms of atherosclerosis. Symptoms of atherosclerosis in heart arteries include, e.g., chest pain, shortness of breath, pressure (angina), pain in the shoulders, back, neck, or arms, dizziness or lightheadedness, heart palpitations, fatigue, nausea or vomiting, or a combination thereof. Symptoms of atherosclerosis in mesenteric arteries include, e.g., abdominal pain or cramping, bloating, nausea, vomiting, diarrhea, unintentional weight loss, or a combination thereof. Symptoms of atherosclerosis in arteries leading to the brain include, e.g., numbness or weakness in the arms or legs, difficulty speaking or slurred speech, temporary loss of vision in one eye, drooping muscles in the face, dizziness, headache, or a combination thereof. These signal a transient ischemic attack (TIA). Untreated, a TIA can lead to a stroke. Symptoms of atherosclerosis in arteries in the arms and legs include, e.g., leg pain when walking (claudication), decreased blood pressure in an affected limb, changes in skin color, cool skin, frequent skin and soft tissue infections, or a combination thereof. Symptoms of atherosclerosis in arteries leading to kidneys include, e.g., high blood pressure, kidney failure, swelling, drowsiness or fatigue, dry or itchy or numb skin, headaches, unexplained weight loss, nausea, vomiting, loss of appetite, or a combination thereof.

The treatment may be one known in the art for atherosclerosis or one described herein. In some aspects, the treatment comprises a TNFAIP2 inhibitor. In some aspects, the TNFAIP2 inhibitor comprises an anti-TNFAIP2 antibody or a TNFAIP2 binding fragment thereof comprising: a V_{L} chain having an amino acid sequence with at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity to SEQ ID NO: 1 and a VII chain having an amino acid sequence with least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity to SEQ ID NO:2; and/or a V_{L} chain having an amino acid sequence with least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity to SEQ ID NO:3 and a V_{H} chain having an amino acid sequence with least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity to SEQ ID NO:4.

In some aspects, the TNFAIP2 inhibitor comprises an anti-TNFAIP2 antibody or a TNFAIP2 binding fragment thereof comprising: a light chain variable region (V_{L}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO: 9, SEQ ID NO: 10, SEQ ID NO:11, SEQ ID NO: 15, SEQ ID NO: 16, or SEQ ID NO: 17; and a heavy chain variable region (V_{H}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO: 12, SEQ ID NO: 13, SEQ ID NO: 14. SEQ ID NO:18, SEQ ID NO: 19, or SEQ ID NO:20.

In some aspects, the TNFAIP2 inhibitor comprises an anti-TNFAIP2 antibody or a TNFAIP2 binding fragment thereof comprising: a light chain variable region (V_{L}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO: 9, SEQ ID NO:10, and SEQ ID NO:11; and a heavy chain variable region (V_{H}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO: 12, SEQ ID NO: 13, and SEQ ID NO: 14.

In some aspects, the TNFAIP2 inhibitor comprises an anti-TNFAIP2 antibody or a TNFAIP2 binding fragment thereof comprising: a light chain variable region (V_{L}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO: 15, SEQ ID NO:16, and SEQ ID NO:17; and a heavy chain variable region (V_{H}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO: 18, SEQ ID NO: 19, and SEQ ID NO:20.

Other treatments useful in the methods of the disclosure include, without limitation, lifestyle changes (e.g., avoiding tobacco products, following a heart-healthy diet, exercise), medications (e.g., those that lower blood pressure or cholesterol, manage blood sugar levels, and/or prevent blood clots) (e.g., ACE inhibitors, beta blockers, anti-platelet or anti-clotting medicines, calcium channel blockers, empagliflozin, canagliflozin, liraglutide, metformin, nitrates, ranolazine, statins, ezetimibe, PCSK9 inhibitor, bempedoic acid, omega-3 fatty acids, thrombolytic medicines), procedures or surgeries (e.g., angioplasty, atherectomy, coronary endarterectomy, carotid endarterectomy, coronary artery bypass grafting (CABG), percutaneous coronary intervention (PCI), peripheral artery bypass, stent placement, vascular disease bypass), or a combination thereof.

Kit aspects of the disclosure may comprise or further comprise one or more negative or positive control samples. In some aspects, the detection agent comprises an anti-TNFAIP2 antibody or a TNFAIP2 binding fragment thereof. In some aspects, the detection agent comprises an anti-TNFAIP2 antibody or a TNFAIP2 binding fragment comprising: a V_{L} chain having an amino acid sequence with least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity to SEQ ID NO:1 and a V_{H} chain having an amino acid sequence with least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity to SEQ ID NO:2; a V_{L} chain having an amino acid sequence with least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity to SEQ ID NO:3; and a V_{H} chain having an amino acid sequence with least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity to SEQ ID NO:4.

In some aspects, the detection agent comprises an anti-TNFAIP2 antibody or a TNFAIP2 binding fragment comprising: a light chain variable region (V_{L}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:9, SEQ ID NO: 10, SEQ ID NO:11, SEQ ID NO: 15, SEQ ID NO: 16, or SEQ ID NO: 17; and a heavy chain variable region (V_{H}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO: 12, SEQ ID NO: 13, SEQ ID NO: 14. SEQ ID NO: 18, SEQ ID NO: 19, or SEQ ID NO:20.

In some aspects, the detection agent comprises an anti-TNFAIP2 antibody or a TNFAIP2 binding fragment comprising: a light chain variable region (V_{L}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:9, SEQ ID NO: 10, and SEQ ID NO:11; and a heavy chain variable region (V_{H}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:12, SEQ ID NO: 13, and SEQ ID NO: 14.

In some aspects, the detection agent comprises an anti-TNFAIP2 antibody or a TNFAIP2 binding fragment comprising: a light chain variable region (V_{L}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:15, SEQ ID NO: 16, and SEQ ID NO:17; and a heavy chain variable region (V_{H}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:18, SEQ ID NO: 19, and SEQ ID NO:20.

In some aspects, the anti-TNFAIP2 antibody or binding fragment is operatively linked to a solid support. In some aspects, the kit comprises at least two anti-TNFAIP2 antibodies, at least two anti-TNFAIP2 antibody binding fragments, or one anti-TNFAIP2 antibody and one anti-TNFAIP2 antibody binding fragment. In some aspects, the one or more anti-TNFAIP2 antibodies or TNFAIP2 antibody binding fragments is linked to a detectable label.

In some aspects, the kit comprises an ELISA for detecting TNFAIP2. In some aspects, the ELISA utilizes an anti-TNFAIP2 antibody or TNFAIP2 binding molecule comprising: an antibody having a V_{L} chain having an amino acid sequence with least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity to SEQ ID NO: 1 and a V_{H} chain having an amino acid sequence with least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity to SEQ ID NO:2; and/or an antibody having a V_{L} chain having an amino acid sequence with least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity to SEQ ID NO:3 and a V_{H} chain having an amino acid sequence with least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity to SEQ ID NO:4.

In some aspects, the ELISA utilizes an anti-TNFAIP2 antibody or TNFAIP2 binding molecule comprising: a light chain variable region (V_{L}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:9, SEQ ID NO: 10, SEQ ID NO:11, SEQ ID NO:15, SEQ ID NO:16, or SEQ ID NO: 17; and a heavy chain variable region (V_{H}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:12, SEQ ID NO:13, SEQ ID NO:14. SEQ ID NO:18, SEQ ID NO: 19, or SEQ ID NO:20.

In some aspects, the ELISA utilizes an anti-TNFAIP2 antibody or TNFAIP2 binding molecule comprising: a light chain variable region (V_{L}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:9, SEQ ID NO: 10, and SEQ ID NO: 11; and a heavy chain variable region (V_{H}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:12, SEQ ID NO:13, and SEQ ID NO: 14.

In some aspects, the ELISA utilizes an anti-TNFAIP2 antibody or TNFAIP2 binding molecule comprising: a light chain variable region (V_{L}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:15, SEQ ID NO: 16, and SEQ ID NO:17; and a heavy chain variable region (V_{H}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:18, SEQ ID NO: 19, and SEQ ID NO:20.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments or aspects discussed elsewhere in a different Example or elsewhere in the application, such as in the Summary of Invention, Detailed Description of the Embodiments, Claims, and description of Figure Legends.

Any method in the context of a therapeutic, diagnostic, or physiologic purpose or effect may also be described in “use” claim language such as “use of” any compound, composition, or agent discussed herein for achieving or implementing a described therapeutic, diagnostic, or physiologic purpose or effect.

Use of the one or more compositions may be employed based on any of the methods described herein. Other embodiments are discussed throughout this application. Any embodiment or aspect discussed with respect to one aspect of the disclosure applies to other aspects and embodiments of the disclosure as well and vice versa.

Other objects, features, and advantages of the present disclosure will become apparent from the following detailed description. It should be understood, however, that the detailed description and the specific examples, while indicating specific aspects of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1I. YAP activation promotes TNFAIP2 expression. FIG. 1A. Cell compartment analysis of gene expression profile of HUVECs overexpressed YAP showing secretory proteins are highly expressed. FIG. 1B. Heat map of YAP-induced expression of secretory proteins. FIG. 1C. The expression of secretory proteins in the endothelium from ApoE−/− mice fed with normal diet (ND) or western diet (WD). FIGS. 1D-1E. Yap overexpression (FIG. 1D) and disturbed flow (DF) (FIG. 1E) induce TNFAIP2 protein secretion. FIG. 1F. DF promotes TNFAIP2 mRNA expression. FIGS. 1G-1H. Laminar shear stress (LSS) (FIG. 1G) and AMPK activators (AICAR and 2-DG) (FIG. 1H) suppressed TNFAIP2 mRNA expression. FIG. 1I. Two tandem TEAD binding sites were identified in the promoter of TNFAIP2. Results are mean±SEM. *p<0.05 (unpaired Student′s t-test).

FIGS. 2A-2F. TNFAIP2 is an early biomarker for atherosclerosis. FIGS. 2A-2C. ApoE−/− mice on a western diet for 0, 1, 3, and 5 months. Plaque formation (FIGS. 2A-2B) was measured by oil-red O staining. Serum TNFAIP2 level (FIG. 2C) was measured by ELISA. FIG. 2D. Serum TNFAIP2 level of C57bl/6 mice and ApoE−/− mice on a normal diet (ND) or western diet (WD) was measured by ELISA. FIG. 2E. TNFAIP2 expression level in aortas of ApoE−/− mice fed on WD for different durations. FIG. 2F. Immunostaining showing the expression of TNFAIP2 in the aortic valve from patients with/without plaques. Results are mean±SEM. *p<0.05 was considered statistically significant (unpaired Student's 1-test).

FIGS. 3A-3H. TNFAIP2 induced monocyte transendosome migration. FIGS. 3A-3B. TNFAIP2 treatment increased macrophage infiltration. FIG. 3C. TNFAIP2 promoted expression of proinflammatory genes. FIG. 3D. GSEA enrichment analysis indicates that TNFAIP2 treatment induced monocyte transendothelial migration. FIG. 3E. Increased expression of genes related to monocyte transendothelial migration. FIG. 3F. CRISPR/Cas9 mediated TNFAIP2 knockdown. FIGS. 3G-3H. TNFAIP2 knockdown suppressed plaque formation as revealed by the representative oil red staining. Results are mean±SEM. *p<0.05 was considered statistically significant (unpaired Student's 1-test).

FIGS. 4A-4F. Centrosome mediates TNFAIP2-induced monocyte transendothelium migration. FIGS. 4A-B. Identification of TNFAIP2 binding protein by BioID2 proximity labeling. Monocytes were treated with TNFAIP2-BioID2 fusion protein (FIG. 4A; lane 1, 12: Marker; lane 2: blank; lane 3: unlabeled TNFAIP2; lane 4, 5, 6: TNFAIP2 N terminal BioID2 fusion protein; lane 7: TNFAIP2 with N terminal BioID2 fusion protein competed with unlabeled TNFAIP2; lane 8, 9, 10: TNFAIP2 with C terminal BioID2 fusion protein). Mass spectrometer identification of specific bands is shown in FIG. 4B. FIG. 4C. Monocyte treated with Cy3 labeled TNFAIP2 revealed TNFAIP2 localization in the perinuclear region and colocalization with CEP250. FIGS. 4D-4E. Enrichment of the gene expression profile of TNFAIP2-treated monocytes. As shown in FIG. 4D, Hallmark enrichment demonstrated TNFAIP2-activated mitotic-spindle and inflammation pathways. As shown in FIG. 4E, BioCarta enrichment demonstrated TNFAIP2-activated PAR1 and TGFB signaling. FIG. 4F. Diagram of TNFAIP2 mediated endothelial cell-monocyte cross-talking.

FIG. 5. ELISA assay for detection of TNFAIP2 in samples using a monoclonal antibody against TNFAIP2. Samples comprised different concentrations of TNFAIP2 prepared in PBST containing 10% FBS.

### DETAILED DESCRIPTION

This disclosure is based, at least in part, on the surprising discovery of TNFAIP2 as a sensitive and reliable biomarker for early diagnosis of atherosclerosis. Yes-associated protein (YAP)-induced secretory proteins in endothelium were assessed as described herein to identify several secretory proteins controlled by YAP, which can be hyperactivated in vascular endothelium early in atherogenesis. Based on the expression profile of endothelial cells (ECs) from atherosclerotic ApoE−/− mice, biomarker targets were narrowed to TNFAIP2. TNFAIP2 was found to be highly expressed in ECs in atherosclerotic plaques. An ELISA kit was constructed for rapid and accurate detection of TNFAIP2, which is more sensitive and convenient compared to other methods of diagnosing atherosclerosis. Suppression of TNFAIP2 was also surprising found to delay the formation of atherosclerotic plaques, suggesting TNFAIP2 as a target for the treatment of atherosclerotic vascular diseases. Accordingly, the novel finding described herein emphasizes TNFAIP2 as a biomarker for early detection, diagnosis, prognosis, and monitoring of atherosclerosis and as a target for treatment of atherosclerosis.

### I. Examples of Definitions

Reference throughout this specification to “one aspect,” “an aspect,” “a particular aspect,” “a related aspect,” “a certain aspect,” “an additional aspect,” or “a further aspect” or combinations thereof means that a particular feature, structure, or characteristic described in connection with the aspect is included in at least one aspect of the present disclosure. Thus, the appearances of the foregoing phrases in various places throughout this specification are not necessarily all referring to the same aspect. Furthermore, the particular features, structures, or characteristics may be combined in any suitable manner in one or more aspects.

Throughout this application, the terms “about” and “approximately” and “substantially” are used according to their plain and ordinary meaning in the area of cell and molecular biology to indicate a deviation of +10% of the value(s) to which it is attached. Therefore, in any disclosed aspect, the terms may be substituted with “within [a percentage] of” what is specified. In one non-limiting aspect, the percentage includes 0.1, 0.5, 1, 5, and 10 percent.

The terms “substantially the same,” “not significantly different,” or “within the range” refers to a level of expression that is not significantly different than what it is compared to. Alternatively, or in conjunction, the terms refer to a level of expression that is less than 2—, 1.5-, or 1.25-fold different or less than 2, 1, or 0.5 standard deviations than the expression or activity level it is compared to.

Recitation of ranges of values herein is merely intended to serve as a shorthand method of referring individually to each separate value falling within the range. Unless otherwise indicated herein, each individual value is incorporated into the specification as if it was individually recited herein.

The use of the word “a” or “an” when used in conjunction with the terms “comprising,” “including,” “having,” or “containing,” or variations of these terms, may mean “one,” but it is also consistent with the meaning of “one or more,” “at least one,” and “one or more than one.”

The use of the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an aspect.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to whatever follows the phrase “consisting of.” Thus, the phrase “consisting of” indicates that the listed elements are required or mandatory, and that no other elements may be present. Compositions and methods “consisting essentially of” any of the ingredients or steps disclosed limits the scope of the claim to any elements listed after the phrase and the specified materials or steps which do not materially affect the basic and novel characteristic of the aspects of the disclosure. Thus, the phrase “consisting essentially of” indicates that the listed elements are required or mandatory, but that other elements are optional and may or may not be present depending upon whether or not they affect the activity or action of the listed elements.

As used herein, unless otherwise indicated, the terms “reference,” “standard,” or “control” describe a value relative to which a comparison is performed. For example, an agent, subject, population, sample, or value of interest is compared with a reference, standard, or control agent, subject, population, sample, or value of interest. A reference, standard, or control may be tested and/or determined substantially simultaneously and/or with the testing or determination of interest for an agent, subject, population, sample, or value of interest and/or may be determined or characterized under comparable conditions or circumstances to the agent, subject, population, sample, or value of interest under assessment.

As used herein, “increased expression” or “elevated expression” or “decreased expression” refers to an expression level of a biomarker in the subject's sample as compared to a reference level representing the same biomarker or a different biomarker. In certain aspects, the reference level may be a reference level of expression from a non-cancerous tissue from the same subject. Alternatively, the reference level may be a reference level of expression from a different subject or group of subjects. For example, the reference level of expression may be an expression level obtained from a sample (e.g., a tissue, fluid or cell sample) of a subject or group of subjects without cancer, with fast doubling time HCC, or with slow doubling time HCC, or an expression level obtained from a non-cancerous tissue of a subject or group of subjects with cancer. The reference level may be a single value or may be a range of values. The reference level of expression can be determined using any method known to those of ordinary skill in the art. The reference level may also be depicted graphically as an area on a graph. In certain embodiments, a reference level is a normalized level.

The term “determining” or “evaluating” as used herein may refer to measuring, detecting, assaying, quantitating, or quantifying (either qualitatively or quantitatively).

The term “subject,” as used herein, can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as atherosclerosis. The subject may be undergoing or having undergone treatment. The subject may be asymptomatic. The subject may be healthy individuals but that are desirous of prevention of atherosclerosis. The term “individual” may be used interchangeably, in at least some cases. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants and includes in utero individuals. It is not intended that the term connote a need for medical treatment, therefore, an individual may voluntarily or involuntarily be part of experimentation whether clinical or in support of basic science studies. In specific aspects, the subject is a human. In specific aspects, the subject is an animal.

As used herein “treatment” or “treating,” includes any beneficial or desirable effect on the symptoms or pathology of a disease or pathological condition and may include even minimal reductions in one or more measurable markers of the disease or condition being treated, e.g., atherosclerosis. Treatment can involve optionally either the reduction, suppression, or amelioration of symptoms of the disease or condition (e.g., plaque formation), or the delaying of the progression of the disease or condition. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood or risk of the occurrence or recurrence of, a disease or condition, e.g., atherosclerosis. It also refers to delaying the onset or recurrence of a disease or condition (e.g., atherosclerosis) or delaying the occurrence or recurrence of the symptoms of a disease or condition (e.g., plaque formation). As used herein, “prevention” and similar words also includes reducing the intensity, effect, symptoms and/or burden of a disease or condition prior to onset or recurrence of the disease or condition.

As will be understood from context, “risk” of a disease, disorder, and/or condition refers to a likelihood that a particular individual will develop the disease, disorder, and/or condition. In some aspects, risk is expressed as a percentage. In some aspects, risk is, is at least, or is at most from 0, 1, 2, 3, 4, 5, 6, 7, 8, 9, 10, 20, 30, 40, 50, 60, 70, 80, 90 up to 100%. In some aspects risk is expressed as a risk relative to a risk associated with a reference sample or group of reference samples. In some aspects, a reference sample or group of reference samples have a known risk of a disease, disorder, condition and/or event. In some aspects a reference sample or group of reference samples are from individuals comparable to a particular individual. In some aspects, risk may reflect one or more genetic attributes, e.g., which may predispose an individual toward development (or not) of a particular disease, disorder and/or condition. In some aspects, risk may reflect one or more epigenetic events or attributes and/or one or more lifestyle or environmental events or attributes. An individual who is “susceptible to” a disease, disorder, and/or condition is one who has a higher risk of developing the disease, disorder, and/or condition than does a member of the general public. In some aspects, an individual who is susceptible to a disease, disorder and/or condition may not have been diagnosed with the disease, disorder, and/or condition. In some aspects, an individual who is susceptible to a disease, disorder, and/or condition may exhibit symptoms of the disease, disorder, and/or condition. In some aspects, an individual who is susceptible to a disease, disorder, and/or condition may not exhibit symptoms of the disease, disorder, and/or condition. In some aspects, an individual who is susceptible to a disease, disorder, and/or condition will develop the disease, disorder, and/or condition. In some aspects, an individual who is susceptible to a disease, disorder, and/or condition will not develop the disease, disorder, and/or condition.

“Prognosis” refers to as a prediction of how a patient will progress, and whether there is a chance of recovery. “Atherosclerosis prognosis” generally refers to a forecast or prediction of the probable course or outcome of the atherosclerosis, with or without a treatment. As used herein, atherosclerosis prognosis includes the forecast or prediction of any one or more of the following: duration of survival of a patient susceptible to or diagnosed with atherosclerosis, duration of recurrence-free survival, duration of progression free survival of a patient susceptible to or diagnosed with atherosclerosis, response rate in a group of patients susceptible to or diagnosed with atherosclerosis, and/or duration of response in a patient or a group of patients susceptible to or diagnosed with atherosclerosis. Prognosis also includes prediction of favorable responses to atherosclerosis treatments, such as a conventional atherosclerosis therapies. A response may be either a therapeutic response (sensitivity or recurrence-free survival) or a lack of therapeutic response (residual disease, which may indicate resistance or recurrence).

A treatment is “therapeutically effective” when it results in a reduction in one or more of the number, severity, and frequency of one or more symptoms of a disease state (e.g., atherosclerosis) in a subject (e.g., a human or an animal).

The phrase “pharmaceutically acceptable” includes compositions that do not produce an allergic or similar untoward reaction when administered to a human or an animal. Typically, such compositions are prepared either as topical compositions, liquid solutions or suspensions, solid forms suitable for solution in, or suspension in, liquid prior to use can also be prepared. Routes of administration can vary with the location and nature of the condition to be treated, and include, e.g., topical, inhalation, intradermal, transdermal, parenteral, intravenous, intramuscular, intranasal, subcutaneous, percutaneous, intratracheal, intraperitoneal, intratumoral, perfusion, lavage, direct injection, and oral administration and formulation.

### II. Atherosclerosis

Provided herein, in some aspects, are methods and compositions for diagnosing, treating, preventing, monitoring, or prognosing atherosclerosis in subjects with particular applications of biomarker expression or activity levels. Atherosclerosis can affect most of the arteries in the body, including arteries in the heart, brain, arms, legs, pelvis, and kidneys. It has different names based on which arteries are affected. In some aspects, the atherosclerosis comprises carotid artery disease, coronary artery disease, heart attack, mesenteric ischemia, peripheral artery disease, renal artery stenosis, stroke, transient ischemic attack (TIA), or a combination thereof, and/or is further characterized as having features described below.

Atherosclerosis is a complex disease that can result from the accumulation of lipids, cholesterol, calcium, and other substances in the artery walls, leading to the formation of plaques that can restrict blood flow or cause a clot upon rupture. Predicting the occurrence of atherosclerosis can be challenging, as it is influenced by a multitude of factors. In non-limiting aspects, biomarkers for early detection, diagnosis, prognosis, and monitoring of atherosclerosis can include TNFAIP2 levels, cholesterol levels, LDL levels, HDL levels, blood pressure, carotid ultrasound, and/or blood glucose levels. In some aspects, TNFAIP2 expression can be combined with other diagnostic biomarkers, e.g., cholesterol levels, LDL levels, HDL levels, blood pressure, and/or blood glucose levels, to improve detection, diagnosis, prognosis, and monitoring of atherosclerosis. Based on a profile of biomarker expression or activity levels, different diagnoses and/or prognoses may be made for different subjects. Based on a profile of biomarker expression or activity levels, different treatments may be prescribed or recommended for different subjects and/or a treatment may be modified for a subject.

In some aspects, a subject is one that has one or more symptoms of atherosclerosis; and/or is being treated for atherosclerosis, will be treated for atherosclerosis, or is currently undergoing treatment for atherosclerosis. In some aspects, 1, 2, 3, 4, 5, or more of TNFAIP2 levels, cholesterol levels, LDL levels, HDL levels, blood pressure, and/or blood glucose levels are measured in a biological sample from the subject as compared to a control. In some aspects, the control comprises a level of TNFAIP2, cholesterol, LDL, HDL, blood pressure, or blood glucose, or any combination thereof, that is representative of a level of TNFAIP2, cholesterol, LDL, HDL, blood pressure, or blood glucose, or any combination thereof, in a biological sample from a subject without atherosclerosis. In some aspects, TNFAIP2, cholesterol, LDL, HDL, blood pressure, or blood glucose, or any combination thereof, is not detected in the sample or the measured level of TNFAIP2, cholesterol, LDL, HDL, blood pressure, or blood glucose, or any combination thereof, is not significantly different from or is less than the control, and a treatment for atherosclerosis is not administered to the subject. In some aspects, the measured level of TNFAIP2, cholesterol, LDL, HDL, blood pressure, or blood glucose, or any combination thereof, is greater than the control, and the subject is administered a treatment for atherosclerosis. In some aspects, the control comprises a level of TNFAIP2, cholesterol, LDL, HDL, blood pressure, or blood glucose, or any combination thereof, that is representative of a level of TNFAIP2, cholesterol, LDL, HDL, blood pressure, or blood glucose, or any combination thereof, in a biological sample from a subject with atherosclerosis. In some aspects, TNFAIP2, cholesterol, LDL, HDL, blood pressure, or blood glucose, or any combination thereof, is not detected in the sample or the measured level of TNFAIP2, cholesterol, LDL, HDL, blood pressure, or blood glucose, or any combination thereof, is not significantly different from or is less than the control, and a treatment for atherosclerosis is not administered to the subject. In some aspects, the measured level of TNFAIP2, cholesterol, LDL, HDL, blood pressure, or blood glucose, or any combination thereof, is not significantly different from or is greater than the control, and the subject is administered a treatment for atherosclerosis.

### A. Symptoms, Causes, & Risk Factors

Symptoms of atherosclerosis as referred to herein may include in heart arteries include, e.g., chest pain, shortness of breath, pressure (angina), pain in the shoulders, back, neck, or arms, dizziness or lightheadedness, heart palpitations, fatigue, nausea or vomiting, or a combination thereof. Symptoms of atherosclerosis in mesenteric arteries include, e.g., abdominal pain or cramping, bloating, nausea, vomiting, diarrhea, unintentional weight loss, or a combination thereof. Symptoms of atherosclerosis in arteries leading to the brain include, e.g., numbness or weakness in the arms or legs, difficulty speaking or slurred speech, temporary loss of vision in one eye, drooping muscles in the face, dizziness, headache, or a combination thereof. These signal a transient ischemic attack (TIA). Untreated, a TIA can lead to a stroke. Symptoms of atherosclerosis in arteries in the arms and legs include, e.g., leg pain when walking (claudication), decreased blood pressure in an affected limb, changes in skin color, cool skin, frequent skin and soft tissue infections, or a combination thereof. Symptoms of atherosclerosis in arteries leading to kidneys include, e.g., high blood pressure, kidney failure, swelling, drowsiness or fatigue, dry or itchy or numb skin, headaches, unexplained weight loss, nausea, vomiting, loss of appetite, or a combination thereof. In some aspects, a subject has one or more of the foregoing symptoms of atherosclerosis. In some aspects, a subject does not have one or more of the foregoing symptoms of atherosclerosis.

Atherosclerosis is a slowly worsening disease that may begin as early as childhood. It may start with damage or injury to the inner layer of an artery. The damage may be caused by, e.g., high blood pressure, high cholesterol, high triglycerides, a type of fat (lipid) in the blood, smoking or chewing tobacco, diabetes, insulin resistance, obesity, inflammation from an unknown cause or from diseases such as arthritis, lupus, psoriasis or inflammatory bowel disease, or a combination thereof. Once the inner wall of an artery is damaged, blood cells and other substances may gather at the injury site and build up in the inner lining of the artery. Over time, fats, cholesterols, and other substances may also collect on the inner walls of the arteries as a plaque or lesion. Plaque can cause the arteries to narrow, blocking blood flow. The plaque can also burst, leading to a blood clot.

Atherosclerosis begins with endothelial damage. The endothelium is a thin lining of cells that covers the inner layer of the artery wall (intima). Many factors can cause endothelial damage, including high LDL cholesterol levels and toxins from tobacco products. The damage triggers chemical processes that cause white blood cells to travel to the injury site. These cells gather and lead to inflammation within the artery. A “fatty streak” is the first visible sign of atherosclerosis. A fatty streak is a yellow streak or patch formed out of dead foam cells at the site of endothelial damage. Foam cells are white blood cells that consume cholesterol and thus appear foamy. Continued foam cell activity causes further damage to your endothelium. Dead foam cells and other debris continue building up, causing the fatty streak to grow into a larger piece of plaque. A fibrous cap (made of smooth muscle cells) forms over the plaque. This cap prevents bits of plaque from breaking off into your bloodstream. As the plaque grows, it gradually narrows the opening of the artery (lumen), resulting in a narrower opening through which blood can flow. Eventually, the plaque may rupture or erode, which can cause a blood clot to form in the artery. Plaque rupture happens when the fibrous cap that covers the plaque breaks open. With plaque erosion, the fibrous cap stays intact, but endothelial cells around the plaque get worn away. Both events lead to the formation of a blood clot. The clot blocks blood flow and can lead to a heart attack or stroke.

Risk factors for atherosclerosis as referred to herein include, e.g., aging, family history of early heart disease, unhealthy diet, diabetes, high blood pressure, high cholesterol, high levels of C-reactive protein (CRP, a marker of inflammation), lack of exercise, obesity, sleep apnea, smoking and other tobacco use, or a combination thereof. In some aspects, a subject has one or more of the foregoing risk factors for developing atherosclerosis. In some aspects, a subject does not have one or more of the foregoing risk factors for developing atherosclerosis.

### B. Diagnosis

To diagnose atherosclerosis or calculate risk for developing atherosclerosis, a healthcare provider may perform a thorough physical exam, including using a stethoscope to listen to the heart and blood flow through the arteries for a whooshing sound called a “bruit,” which may indicate the presence of plaque. The healthcare provide may also ask about medical and family history and lifestyle factors (e.g., diet, past or present use of tobacco products, etc.). The healthcare provide may also order blood tests to check levels of, e.g., cholesterol, triglyceride, blood sugar, lipoproteins, or proteins that are signs of inflammation, such as C-reactive protein. Other tests that may be ordered include, e.g., angiography, ankle/brachial index, chest x-ray, CT scan, coronary calcium scan, magnetic resonance angiography imaging, positron emission tomography imaging, echocardiogram, electrocardiogram, exercise stress test, carotid ultrasound, abdominal ultrasound, or a combination thereof.

An atherosclerosis diagnosis according to particular applications of biomarker expression or activity levels referred to herein may be confirmed according to any of the foregoing methods.

### C. Prevention and Treatment

Methods of the disclosure relate to treating subjects and patients with a therapy for atherosclerosis. The atherosclerosis therapy may be one described below and may be given with respect to a subject having been determined to have a certain biomarker profile. For example, in some aspects, the therapy described below is given to a subject with a poor prognosis, unfavorable prognosis, or to a subject determined to be high risk. In some aspects, the therapy described below is given to a subject with a favorable prognosis, or to a subject determined to be low risk. Also contemplated are combinations of the therapies described below.

In some aspects, treatment or prevention of atherosclerosis comprises administration of a TNFAIP2 inhibitor or a composition thereof. In some aspects, the TNFAIP2 inhibitor is an antibody. TNFAIP2 inhibitors include any compound or molecule (e.g., small molecule, peptide, peptidomimetic, natural compound, siRNA, anti-sense nucleic acid, aptamer, or antibody) that interferes with (e.g., reduces, decreases, suppresses, eliminates, or blocks) the function of the molecule or pathway. A TNFAIP2 inhibitor can be any compound or molecule that changes any activity of a particular TNFAIP2 molecule, or any molecule involved with the particular TNFAIP2 molecule. In some aspects, the TNFAIP2 inhibitor is a small molecule. In some aspects, the TNFAIP2 inhibitor is an antibody. In some aspects, the TNFAIP2 inhibitor is a nucleic acid.

In some aspects, treatment or prevention of atherosclerosis comprises administration of a TNFAIP2 inhibitor comprising an anti-TNFAIP2 antibody or a TNFAIP2 binding fragment thereof. In some aspects, the TNFAIP2 inhibitor comprising an anti-TNFAIP2 antibody or a TNFAIP2 binding fragment thereof used for treatment or prevention of atherosclerosis comprises: a light chain variable region (V_{L}) having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO: 1 or 3, and a heavy chain variable region (V_{H}) having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:2 or 4. In some aspects, the V_{L} has at least 85% identity to SEQ ID NO: 1 or 3. In some aspects, the V_{H} has at least 85% identity to SEQ ID NO:2 or 4. In some aspects, the V_{L} has at least 90% identity to SEQ ID NO: 1 or 3. In some aspects, the V_{H} has at least 90% identity to SEQ ID NO:2 or 4. In some aspects, the V_{L} has at least 95% identity to SEQ ID NO: 1 or 3. In some aspects, the V_{H} has at least 95% identity to SEQ ID NO:2 or 4. In some aspects, the V_{L} comprises SEQ ID NO: 1 or 3. In some aspects, the V_{H} comprises SEQ ID NO: 2 or 4. In some aspects, the V_{L} has at least 85% identity to SEQ ID NO:1 and the V_{H} has at least 85% identity to SEQ ID NO:2. In some aspects, the V_{L} has at least 90% identity to SEQ ID NO: 1 and the V_{H} has at least 90% identity to SEQ ID NO:2. In some aspects, the V_{L} has at least 95% identity to SEQ ID NO: 1 and the V_{H} has at least 95% identity to SEQ ID NO:2. In some aspects, the V_{L} comprises SEQ ID NO: 1 and the VI comprises SEQ ID NO:2. In some aspects, the V_{L} has at least 85% identity to SEQ ID NO:3 and the V_{H} has at least 85% identity to SEQ ID NO:4. In some aspects, the V_{L} has at least 90% identity to SEQ ID NO:3 and the VII has at least 90% identity to SEQ ID NO:4. In some aspects, the V_{L} has at least 95% identity to SEQ ID NO:3 and the V_{H} has at least 95% identity to SEQ ID NO:4. In some aspects, the V_{L} comprises SEQ ID NO:3 and the VII comprises SEQ ID NO:4.

In some aspects, the TNFAIP2 inhibitor comprising an anti-TNFAIP2 antibody or a TNFAIP2 binding fragment thereof used for treatment or prevention of atherosclerosis comprises: a light chain variable region (V_{L}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:9, SEQ ID NO:10, SEQ ID NO:11, SEQ ID NO: 15, SEQ ID NO:16, or SEQ ID NO:17; and a heavy chain variable region (V_{H}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO: 12, SEQ ID NO:13, SEQ ID NO:14. SEQ ID NO:18, SEQ ID NO:19, or SEQ ID NO:20.

In some aspects, the TNFAIP2 inhibitor comprising an anti-TNFAIP2 antibody or a TNFAIP2 binding fragment thereof used for treatment or prevention of atherosclerosis comprises: a light chain variable region (V_{L}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:9, SEQ ID NO:10, and SEQ ID NO:11; and a heavy chain variable region (V_{H}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:12, SEQ ID NO: 13, and SEQ ID NO: 14.

In some aspects, the TNFAIP2 inhibitor comprising an anti-TNFAIP2 antibody or a TNFAIP2 binding fragment thereof used for treatment or prevention of atherosclerosis comprises: a light chain variable region (V_{L}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:15, SEQ ID NO:16, and SEQ ID NO:17; and a heavy chain variable region (V_{H}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO: 18, SEQ ID NO:19, and SEQ ID NO:20.

Additional TNFAIP2 inhibitors may be identified and administered for treatment or prevention of atherosclerosis. For example, thermal shift assays, also known as protein thermal shift assays or differential scanning fluorimetry (DSF), may be utilized to identify TNFAIP2 inhibitors. Thermal shift assays are rapid, simple, and inexpensive methods for identification of potential drug candidates that bind and stabilize target proteins. The technique is based on the principle that the thermal stability of a protein changes when it interacts with a ligand (such as a small-molecule drug or a protein partner), leading to a change in its melting temperature (Tm). The following are the four steps for thermal shift assays. First, protein-ligand interactions are investigated. A purified protein sample is mixed with a potential ligand. If the ligand binds to the protein, it will change the protein's structure and stability. Second, thermal denaturation is assessed. The protein-ligand mixture is subjected to a range of temperatures to denature (unfold) the protein. This process is monitored in real-time using a fluorescent dye that binds to the exposed hydrophobic regions of the denatured protein. Third, changes in fluorescence is monitored. The fluorescence intensity increases as the protein unfolds and the dye binds, which allows the measurement of the protein's thermal denaturation curve. The midpoint of this transition is defined as the melting temperature (Tm) of the protein. Fourth, thermal shift is measured. The difference between the melting temperatures of the protein in the presence and absence of the ligand (the thermal shift) is calculated. A positive thermal shift indicates that the ligand stabilizes the protein and therefore likely binds to it. Any one or more TNFAIP2 inhibitors identified by thermal shift assay or other such assays known to be useful for identification of potential drug candidates are contemplated to be useful in the context of the present disclosure.

TNFAIP2 inhibitors may act via directly inhibiting TNFAIP2 signaling, inhibiting TNFAIP2 mRNA, causing conformational changes of TNFAIP2, decreasing TNFAIP2 protein levels, or interfering with TNFAIP2 interactions with signaling partners, and affecting the expression of TNFAIP2 target genes. An inhibitor may diminish or decrease the pathway to be inhibited by 10%, 20%, 30%, 40%, 50%, 60%, 70%, 80%, 90%, 100% or more when compared to the activity of the pathway without or before the addition of the inhibitor.

In some aspects, the TNFAIP2 inhibitors inhibit TNFAIP2 activity. In some aspects, the TNFAIP2 inhibitors inhibit monocyte adhesion, infiltration, and differentiation. In some aspects, the TNFAIP2 inhibitors inhibit plaque formation. In some aspects, TNFAIP2 inhibitors inhibit induction of genes related to inflammation and cell migration. In some aspects, TNFAIP2 inhibitors inhibit the mitotic spindle pathway and/or TNFAIP2-induced transendothelial migration. In some aspects, TNFAIP2 inhibitors inhibit activation of the PAR1 pathway and/or TNFAIP2-induced cell polarized migration. The TNFAIP2 inhibitors or compositions thereof may or may not be tailored to address any symptom of atherosclerosis. The compositions may be given to a subject without having prior analysis of TNFAIP2 levels. The compositions may comprise any one or more TNFAIP2 inhibitors associated with efficacious therapy to treat or prevent atherosclerosis. The TNFAIP2 inhibitors or compositions thereof can be administered alone or in combination with one or more additional atherosclerosis therapies comprising but not limited to lifestyle modifications, medications, procedures or surgeries, or a combination thereof.

In some aspects, treatment or prevention of atherosclerosis comprises or further comprises lifestyle modifications, such as quitting smoking, eating healthy foods (e.g., a diet low in saturated fat, trans fat, cholesterol, sodium (salt) and sugar, exercising regularly, maintaining a healthy weight, managing stress, checking and maintaining a healthy blood pressure, checking and maintaining healthy cholesterol and blood sugar levels, or a combination thereof.

In some aspects, treatment or prevention of atherosclerosis comprises or further comprises administration of medications (e.g., those that lower blood pressure or cholesterol, manage blood sugar levels, and/or prevent blood clots) (e.g., ACE inhibitors, beta blockers, anti-platelet or anti-clotting medicines, calcium channel blockers, empagliflozin, canagliflozin, liraglutide, metformin, nitrates, ranolazine, statins, ezetimibe, PCSK9 inhibitor, bempedoic acid, omega-3 fatty acids, thrombolytic medicines).

In some aspects, treatment or prevention of atherosclerosis comprises or further comprises procedures or surgeries (e.g., angioplasty, atherectomy, coronary endarterectomy, carotid endarterectomy, coronary artery bypass grafting (CABG), percutaneous coronary intervention (PCI), peripheral artery bypass, stent placement, vascular disease bypass), or a combination thereof.

Treatment or prevention of atherosclerosis may include or exclude any of the foregoing lifestyle modifications, medications, or procedures or surgeries.

### D. Monitoring

In certain aspects, the biomarker-based methods described herein may be combined with one or more other atherosclerosis diagnosis or screening tests at increased frequency if the subject is determined to be at high risk for plaque formation or have a poor prognosis based on the biomarker as described herein.

In some aspects, the methods of the disclosure further include one or more monitoring tests. The monitoring protocol may include any methods known in the art. In particular, the monitoring can include obtaining a sample and testing the sample for diagnosis. For example, the monitoring may include blood tests to check levels of, e.g., cholesterol, triglyceride, blood sugar, lipoproteins, proteins that are signs of inflammation, such as C-reactive protein, or a combination thereof, or other tests including, e.g., angiography, ankle/brachial index, chest x-ray, CT scan, coronary calcium scan, magnetic resonance angiography imaging, positron emission tomography imaging, echocardiogram, electrocardiogram, exercise stress test, carotid ultrasound, abdominal ultrasound, or a combination thereof.

### E. ROC Analysis

In statistics, a receiver operating characteristic (ROC), or ROC curve, is a graphical plot that illustrates the performance of a binary classifier system as its discrimination threshold is varied. ROC analysis may be applied to determine a cut-off value or threshold setting of biomarker expression. For example, subjects with biological samples determined to have a biomarker expression value above a certain cut-off threshold but below a higher cut-off threshold may be determined to have atherosclerosis. Subjects with biological samples determined to have a biomarker expression level that surpasses the cut-off threshold for atherosclerosis may be determined to have atherosclerosis.

The curve is created by plotting the true positive rate against the false positive rate at various threshold settings. The true-positive rate is also known as sensitivity in biomedical informatics. The false-positive rate is also known as the fall-out and can be calculated as 1-specificity. The ROC curve is thus the sensitivity as a function of fall-out. In general, if the probability distributions for both detection and false alarm are known, the ROC curve can be generated by plotting the cumulative distribution function (area under the probability distribution from −infinity to +infinity) of the detection probability in the y-axis versus the cumulative distribution function of the false-alarm probability in x-axis.

ROC analysis provides tools to select possibly optimal models and to discard suboptimal ones independently from (and prior to specifying) the cost context or the class distribution. ROC analysis is related in a direct and natural way to cost/benefit analysis of diagnostic decision making.

The ROC is also known as a relative operating characteristic curve because it is a comparison of two operating characteristics (TPR and FPR) as the criterion changes. ROC analysis curves are known in the art and described in Metz CE (1978). Basic principles of ROC analysis. Seminars in Nuclear Medicine 8:283-298; Youden WJ (1950). An index for rating diagnostic tests. Cancer 3:32-35; Zweig M H, Campbell G (1993). Receiver-operating characteristic (ROC) plots: a fundamental evaluation tool in clinical medicine. Clinical Chemistry 39:561-577; and Greiner M, Pfeiffer D, Smith RD (2000). Principles and practical application of the receiver-operating characteristic analysis for diagnostic tests. Preventive Veterinary Medicine 45:23-41, which are herein incorporated by reference in their entirety. A ROC analysis may be used to create cut-off values for prognosis and/or diagnosis purposes according to some methods disclosed herein.

### III. Protein Assays

A variety of techniques can be employed to measure expression levels of polypeptides and proteins in a biological sample to determine biomarker expression levels. Examples of such formats include, but are not limited to, enzyme immunoassay (EIA), radioimmunoassay (RIA), Western blot analysis, and enzyme linked immunosorbent assay (ELISA). A skilled artisan can readily adapt known protein/antibody detection methods for use in determining protein expression levels of biomarkers.

In one aspect, antibodies, or antibody fragments or derivatives, can be used in methods such as Western blots, ELISA, or immunofluorescence techniques to detect biomarker expression. As used herein, the term “antibody” encompasses antibodies and antibody fragments thereof, derived from any antibody-producing mammal (e.g., mouse, rat, rabbit, and primate including human), that specifically bind to an antigenic polypeptide. Exemplary antibodies include polyclonal, monoclonal and recombinant antibodies; multispecific antibodies (e.g., bispecific antibodies); humanized antibodies; murine antibodies; chimeric, mouse-human, mouse-primate, primate-human monoclonal antibodies; and anti-idiotype antibodies, and may be any intact molecule or fragment thereof.

In some aspects, either the antibodies or proteins are immobilized on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present disclosure. The support can then be washed with suitable buffers followed by treatment with the detectably labeled antibody. The solid phase support can then be washed with the buffer a second time to remove unbound antibody. The amount of bound label on the solid support can then be detected by conventional means.

Immunohistochemistry methods are also suitable for detecting the expression levels of biomarkers. In some aspects, antibodies or antisera, including polyclonal antisera, and monoclonal antibodies specific for each marker may be used to detect expression. The antibodies can be detected by direct labeling of the antibodies themselves, for example, with radioactive labels, fluorescent labels, hapten labels such as, biotin, or an enzyme such as horseradish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

Immunological methods for detecting and measuring complex formation as a measure of protein expression using either specific polyclonal or monoclonal antibodies are known in the art. Examples of such techniques include enzyme-linked immunosorbent assays (ELISAs), radioimmunoassays (RIAs), fluorescence-activated cell sorting (FACS), antibody arrays, proximity ligation assays, proximity extension assays, and PCR-ELISAs. Such immunoassays typically involve the measurement of complex formation between the protein and its specific antibody. These assays and their quantitation against purified, labeled standards are well known in the art. A two-site, monoclonal-based immunoassay utilizing antibodies reactive to two non-interfering epitopes or a competitive binding assay may be employed.

Numerous labels are available and commonly known in the art. Radioisotope labels include, for example, 36S, 14C, 1251, 3H, and 1311. The antibody can be labeled with the radioisotope using the techniques known in the art. Fluorescent labels include, for example, labels such as rare earth chelates (europium chelates) or fluorescein and its derivatives, rhodamine and its derivatives, dansyl, Lissamine, phycoerythrin and Texas Red are available. The fluorescent labels can be conjugated to the antibody variant using the techniques known in the art. Fluorescence can be quantified using a fluorimeter. Various enzyme-substrate labels are available and U.S. Pat. Nos. 4,275,149, 4,318,980 provides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, .beta.-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for Use in Enzyme Immunoassay, in Methods in Enzymology (Ed. J. Langone & H. Van Vunakis), Academic press, New York, 73:147-166 (1981).

In some aspects, a detection label is indirectly conjugated with an antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). In some aspects, the antibody need not be labeled, and the presence thereof can be detected using a labeled antibody, which binds to the antibody.

Other methods for biomarker detection in a biological sample from a subject contemplated for use according to the methods disclosed herein include proximity ligation assays, proximity extension assays, and colloidal gold immunochromatographic strips assay.

### IV. Antibodies

Aspects of the disclosure relate to TNFAIP2-binding proteins. The terms “polypeptide that specifically binds TNFAIP2” and “TNFAIP2-binding protein” are used interchangeably herein. In some aspects, the TNFAIP2-binding protein specifically binds to human TNFAIP2.

In some aspects, the TNFAIP2-binding protein is an antibody, an antibody-like molecule, or an antigen-binding fragment thereof. In some aspects, the TNFAIP2-binding protein is an antibody, a nanobody, a minibody, an scFv fragment, or a Fab fragment. In some aspects, the TNFAIP2-binding protein is a human antibody, humanized antibody, recombinant antibody, chimeric antibody, an antibody derivative, a veneered antibody, a diabody, a monoclonal antibody, or a polyclonal antibody. In some aspects, the TNFAIP2-binding protein is a monoclonal antibody. In some aspects, the TNFAIP2-binding protein is a murine antibody. In some aspects, the TNFAIP2-binding protein is a chimeric antibody. In some aspects, the TNFAIP2-binding protein is a humanized antibody.

In some aspects, an anti-TNFAIP2 antibody or a TNFAIP2 binding fragment thereof according to any one or more of the methods and compositions disclosed herein comprises a light chain variable region (V_{L}) having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:1 or 3, and a heavy chain variable region (V_{H}) having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:2 or 4. In some aspects, the V_{L} has at least 85% identity to SEQ ID NO: 1 or 3. In some aspects, the V_{H} has at least 85% identity to SEQ ID NO:2 or 4. In some aspects, the V_{L} has at least 90% identity to SEQ ID NO: 1 or 3. In some aspects, the V_{H} has at least 90% identity to SEQ ID NO:2 or 4. In some aspects, the V_{L} has at least 95% identity to SEQ ID NO:1 or 3. In some aspects, the V_{H} has at least 95% identity to SEQ ID NO:2 or 4. In some aspects, the V_{L} comprises SEQ ID NO:1 or 3. In some aspects, the V_{H} comprises SEQ ID NO: 2 or 4. In some aspects, the V_{L} has at least 85% identity to SEQ ID NO: 1 or 3 and the Vu has at least 85% identity to SEQ ID NO:2 or 4. In some aspects, the V_{L} has at least 90% identity to SEQ ID NO: 1 or 3 and the V_{H} has at least 90% identity to SEQ ID NO:2 or 4. In some aspects, the V_{L} has at least 95% identity to SEQ ID NO: 1 or 3 and the V_{H} has at least 95% identity to SEQ ID NO:2 or 4. In some aspects, the V_{L} comprises SEQ ID NO: 1 or 3 and the V_{H} comprises SEQ ID NO:2 or 4.

In some aspects, an anti-TNFAIP2 antibody or a TNFAIP2 binding fragment thereof according to any one or more of the methods and compositions disclosed herein comprises: a light chain variable region (V_{L}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:9, SEQ ID NO:10, SEQ ID NO:11, SEQ ID NO: 15, SEQ ID NO:16, or SEQ ID NO:17; and a heavy chain variable region (V_{H}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO: 12, SEQ ID NO:13, SEQ ID NO: 14. SEQ ID NO:18, SEQ ID NO: 19, or SEQ ID NO:20.

In some aspects, an anti-TNFAIP2 antibody or a TNFAIP2 binding fragment thereof according to any one or more of the methods and compositions disclosed herein comprises: a light chain variable region (V_{L}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:9, SEQ ID NO:10, and SEQ ID NO:11; and a heavy chain variable region (V_{H}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO: 12, SEQ ID NO: 13, and SEQ ID NO: 14.

In some aspects, an anti-TNFAIP2 antibody or a TNFAIP2 binding fragment thereof according to any one or more of the methods and compositions disclosed herein comprises: a light chain variable region (V_{L}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:15, SEQ ID NO:16, and SEQ ID NO:17; and a heavy chain variable region (V_{H}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO: 18, SEQ ID NO: 19, and SEQ ID NO:20.

As used herein, the term “antibody” refers to an intact immunoglobulin of any class or isotype, or a fragment thereof that can compete with the intact antibody for specific binding to the target antigen. An isotype refers to the genetic variations or differences in the constant regions of the heavy and light chains of an antibody. In humans, there are five heavy chain isotypes: IgA, IgD, IgG, IgE, and IgM and two light chain isotypes: kappa and lambda. The IgG class is divided into four isotypes: IgG1, IgG2, IgG3 and IgG4 in humans, and IgG1, IgG2a, IgG2b and IgG3 in mice. They share more than 95% homology in the amino acid sequences of the Fc regions but show major differences in the amino acid composition and structure of the hinge region.

The term “antibody” includes a polyclonal antibody, a monoclonal antibody, a chimeric antibody, a human antibody, a veneered antibody, a diabody, a humanized antibody, an antibody derivative, a recombinant antibody, a recombinant humanized antibody, an engineered antibody, a multi-specific antibody, a DARPin, or a derivative or fragment of each thereof. Also contemplated are antibodies having specificity for more than one antigen or target, including bispecific antibodies, trispecific antibodies, tetraspecific antibodies, and other multispecific antibodies.

As used herein, an “antibody” includes whole antibodies and any antigen binding fragment or a single chain thereof. Thus the term “antibody” includes any protein or peptide containing molecule that comprises at least a portion of an immunoglobulin molecule. As used herein, the terms “antibody” or “immunoglobulin” are used interchangeably and refer to any of several classes of structurally related proteins that function as part of the immune response of an animal, including IgM, IgD, IgG, IgA, IgE, and related proteins, as well as polypeptides comprising antibody CDR domains that retain antigen-binding activity. Examples of such include but are not limited to a complementarity determining region (CDR) of a heavy or light chain or a ligand binding portion thereof, a heavy chain or light chain variable region, a heavy chain or light chain constant region, a framework (FR) region or any portion thereof or at least one portion of a binding protein. In certain aspects, the antibody or antigen binding fragment specifically binds human TNFAIP2.

The term “antigen” refers to a molecule or a portion of a molecule capable of being bound by a selective binding agent, such as an antibody. An antigen may possess one or more epitopes that can interact with different antibodies.

The term “epitope” refers to a moiety that is specifically recognized by an immunoglobulin (e.g., antibody or receptor) binding component. Epitope determinants may include a plurality of chemical atoms or groups on an antigen surface, such as amino acids, sugar side chains, phosphoryl, or sulfonyl groups. In some aspects, such chemical atoms or groups are surface-exposed when the antigen adopts a relevant three-dimensional conformation. In some aspects, such chemical atoms or groups are physically near to each other in space when the antigen adopts such a conformation. In some aspects, at least some such chemical atoms or groups are physically separated from one another when the antigen adopts an alternative conformation (e.g., is linearized). Generally, antibodies specific for a particular target antigen would recognize an epitope on the target antigen within a complex mixture.

The epitope regions of a given polypeptide can be identified using many different epitope mapping techniques well known in the art, including: x-ray crystallography, nuclear magnetic resonance spectroscopy, site-directed mutagenesis mapping, protein display arrays, and hydrogen-deuterium exchange see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986), each of which is incorporated by reference herein in their entirety. Additionally, antigenic regions of proteins can also be predicted and identified using standard antigenicity and hydropathy plots.

The term “immunogenic sequence” means a molecule that includes an amino acid sequence of at least one epitope such that the molecule can stimulate the production of antibodies in an appropriate host. The term “immunogenic composition” means a composition that comprises at least one immunogenic molecule (e.g., an antigen or carbohydrate).

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, for chimeric antibodies, the variable regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front Immunol. 2013; 4:302; 2013).

The term “variable region” refers to a portion of the antibody that gives the antibody its specificity for binding antigen. The variable region is typically located at the ends of the heavy and light chains. Variable loops of β-strands, three each on the light (V_{L}) and heavy (V_{H}) chains are responsible for binding to the antigen. These loops are referred to as the “complementarity determining regions” (CDRs). In general, the CDR residues are directly and most substantially involved in influencing antigen binding.

The term “constant region” refers to a portion of the antibody that is identical in all antibodies of the same isotype. The constant region differs in antibodies of different isotypes.

The term “light chain” may describe a full-length light chain or fragments thereof. A full-length light chain has a molecular weight of around 25,000 Daltons and includes a variable region domain (abbreviated herein as V_{L}), and a constant region domain (abbreviated herein as C_{L}). There are two classifications of light chains, identified as kappa (κ) and lambda (2). The term “V_{L} fragment” means a fragment of the light chain of a monoclonal antibody that includes all or part of the light chain variable region, including CDRs. A V_{L} fragment can further include light chain constant region sequences. The variable region domain of the light chain is at the amino-terminus of the polypeptide.

The term “heavy chain” may describe a full-length heavy chain or fragments thereof. For example, a full-length heavy chain for human IgG1 has a molecular weight of around 50,000 Daltons and includes a variable region domain (abbreviated herein as V_{H}), and three constant region domains (abbreviated herein as C_{H}1, C_{H}2, and C_{H}3). The term “V_{H} fragment” means a fragment of the heavy chain of a monoclonal antibody that includes all or part of the heavy chain variable region, including CDRs. A V_{H} fragment can further include heavy chain constant region sequences. The number of heavy chain constant region domains will depend on the isotype. The isotype of an antibody can be IgM, IgD, IgG, IgA, or IgE and is defined by the heavy chains present of which there are five classifications: mu (μ), delta (d), gamma (γ), alpha (α), or epsilon (ε) chains, respectively. Human IgG has several subtypes, including, IgG1, IgG2, IgG3, and IgG4.

### A. Types of Antibodies

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins.

The term “monomer” means an antibody containing only one immunoglobulin unit. Monomers are the basic functional units of antibodies. The term “dimer” means an antibody containing two immunoglobulin units attached to one another via constant domains of the antibody heavy chains (the Fc, or fragment crystallizable, region). The complex may be stabilized by a joining (J) chain protein. The term “multimer” means an antibody containing more than two immunoglobulin units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities, or they may be bi-specific, meaning the two antigen-binding sites have different antigen specificities.

Bispecific antibodies are a class of antibodies that have paratopes (i.e., antigen-binding sites) for two or more distinct epitopes. Bispecific antibodies can be biparatopic, wherein a bispecific antibody may specifically recognize a different epitope from the same antigen. Bispecific antibodies can be constructed from a pair of different single domain antibodies termed “nanobodies.” Single domain antibodies may be sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as a single chain variable fragment (scFv). Diabodies and scFvs can be constructed without an Fc region, using only variable domains. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

In certain aspects, the antigen-binding domain may be multispecific or heterospecific by multimerizing with V_{H} and V_{L} region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

Multispecific antibodies can be used and directly linked via a short flexible polypeptide chain, using routine methods known in the art. One such example is diabodies that are bivalent, bispecific antibodies with two antigen-binding sites in which the V_{H} and V_{L} domains are expressed on a single polypeptide chain and utilize a linker that is too short to allow for pairing between domains on the same chain, thereby forcing the domains to pair with complementary domains of another chain creating two antigen binding sites. The linker functionality is applicable for aspects of triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001), each of which is incorporated herein by reference in their entirety).

The part of the Fv fragment of an antibody molecule that binds with high specificity to the epitope of the antigen is referred to herein as the “paratope.” The paratope consists of the amino acid residues that contact the epitope of an antigen to facilitate antigen recognition. Each of the two Fv fragments of an antibody is composed of the two variable domains, VI and V_{L}, in dimerized configuration. The primary structure of each of the variable domains includes three hypervariable loops separated by, and flanked by, framework regions (FRs). The hypervariable loops are the regions of highest primary sequences variability among the antibody molecules from any mammal. The term hypervariable loop is sometimes used interchangeably with the term “complementarity determining region” (CDR). The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the V_{L} domain are identified as L1, L2, and L3, with L1 occurring at the most distal end and L3 occurring closest to the C_{L} domain. The CDRs may also be given the names CDR-L1, CDR-L2, and CDR-L3. The L3 (CDR-L3) is generally the region of highest variability among all antibody molecules produced by a given organism. The CDRs are regions of the polypeptide chain arranged linearly in the primary structure and separated from each other by FRs. The amino terminal (N-terminal) end of the V_{L} chain is named FR1. The region identified as FR2 occurs between L1 and L2 hypervariable loops. FR3 occurs between L2 and L3 hypervariable loops, and the FR4 region is closest to the C_{L} domain. This structure and nomenclature is repeated for the VII chain, which includes three CDRs identified as H1, H2, and H3, or CDR-H1, CDR-H2 and CDR-H3. The majority of amino acid residues in the variable domains, or Fv fragments (V_{H} and V_{L}), are part of the FRs (approximately 85%).

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, J Exp Med, 132 (2): 211-50 (1970)); Chothia (as described in C. Chothia et al., Nature, 342 (6252): 877-83 (1989)); and IMGT (as described in M.-P. Lefranc et al., Developmental & Comparative Immunology, 27 (1): 55-77 (2003)). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding. Depending on the type and size of the antigen, different CDR residues may contact the antigen. See Almagro JC. J Mol Recognit. 17 (2): 132-43 (2004), incorporated herein by reference.

One skilled in the art can use any of several methods to determine the paratope of an antibody. These methods include:
1) Computational predictions of the tertiary structure of the antibody/epitope binding interactions based on the chemical nature of the amino acid sequence of the antibody variable region and composition of the epitope.
2) Hydrogen-deuterium exchange and mass spectroscopy.
3) Polypeptide fragmentation and peptide mapping approaches in which one generates multiple overlapping peptide fragments from the full length of the polypeptide and evaluates the binding affinity of these peptides for the epitope.
4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific V_{H} and V_{L} domains as appropriate.

In certain aspects, affinity matured antibodies are enhanced with one or more modifications in one or more CDRs thereof (and/or one or more FRs thereof) that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio Technology 10:779 (1992) describes affinity maturation by V_{H} and V_{L} domain shuffling, random mutagenesis of CDR and/or framework residues employed in phage display is described by Rajpal et al., PNAS. 24:8466-8471 (2005) and Thie et al., Methods Mol Biol. 525:309-22 (2009) in conjugation with computation methods as demonstrated in Tiller et al., Front. Immunol. 8:986 (2017), each of which references are incorporated herein by reference in their entirety.

Chimeric immunoglobulins are the products of fused genes derived from different species (the various domains of the antibodies' heavy and light chains are coded for by DNA from more than one species; see, e.g., U.S. Pat. No. 4,816,567); “humanized” antibodies generally have the FRs from human immunoglobulins and one or more CDRs are from a non-human source (e.g., murine).

As used herein, the term “humanized antibody” or “humanized immunoglobulin” refers to a human/non-human chimeric antibody that contains a minimal sequence derived from non-human immunoglobulin. For the most part, humanized antibodies are human immunoglobulins (recipient antibody) in which residues from a variable region of the recipient are replaced by residues from a variable region of a non-human species (donor antibody) such as mouse, rat, rabbit, or non-human primate having the desired specificity, affinity and capacity. Humanized antibodies may comprise residues that are not found in the recipient antibody or in the donor antibody. The humanized antibody can optionally also comprise at least a portion of an immunoglobulin constant region (Fc), typically that of a human immunoglobulin, a non-human antibody containing one or more amino acids in a framework region, a constant region or a CDR, that have been substituted with a correspondingly positioned amino acid from a human antibody. In general, humanized antibodies are expected to produce a reduced immune response in a human host, as compared to a non-humanized version of the same antibody. The humanized antibodies may have conservative amino acid substitutions which have substantially no effect on antigen binding or other antibody functions. Conservative substitutions groupings include: glycine-alanine, valine-leucine-isoleucine, phenylalanine-tyrosine, lysine-arginine, alanine-valine, serine-threonine and asparagine-glutamine. Humanization or engineering of antibodies can be performed using any known method such as, but not limited to, those described in U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567; each incorporated by reference herein in its entirety.

Minimizing the antibody polypeptide sequence from the non-human species can optimize chimeric antibody function and reduces immunogenicity. Specific amino acid residues of the non-human antibody are modified to be homologous to corresponding residues in a human antibody. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. In some instances, corresponding non-human (e.g., murine) residues replace framework region residues of the human immunoglobulin. Replacement of human framework region residues with non-human framework region residues may serve to improve and/or restore antigen binding. Furthermore, humanized antibodies may comprise residues that are not found in the recipient antibody or in the donor antibody to further refine performance. The humanized antibody may also comprise at least a portion of an immunoglobulin constant region, typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhoeyen et al., Science 239:1534-36 (1988); Almagro et al., Front Immunol 8; 1751 (2018); and Payes et al., “Genetic Engineering of Antibody Molecules,” In: Reviews in Cell Biology and Molecular Medicine. John Wiley & Sons, Inc., Hoboken, New Jersey, USA. 1 (3): 1-52 (2015), each of which is incorporated by reference herein in its entirety.

Intrabodies are intracellularly localized immunoglobulins that bind to intracellular antigens as opposed to secreted antibodies, which bind antigens in the extracellular space.

Antibodies also include “linear antibodies.” The procedure for making linear antibodies is known in the art and described in Zapata et al., 1995. Briefly, these antibodies comprise a pair of tandem Ed segments (V_{H}-C_{H}1-V_{H}—C_{H}1) which form a pair of antigen binding regions. Linear antibodies can be bispecific or monospecific.

The terms “polyclonal antibody” or “polyclonal antibody composition” as used herein refer to a preparation of antibodies that are derived from different B-cell lines. They are a mixture of immunoglobulin molecules secreted against a specific antigen, each recognizing a different epitope. Thus, polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

A monoclonal antibody or “mAb” refers to an antibody obtained from a population of substantially homogeneous antibodies from an exclusive parental cell, e.g., the population is identical except for naturally occurring mutations that may be present in minor amounts. Each monoclonal antibody is directed against a single antigenic determinant (epitope). Monoclonal antibodies are highly specific, as each monoclonal antibody is directed against a single determinant on the antigen.

### B. Functional Antibody Fragments and Antigen-Binding Fragments

### 1. Antigen-Binding Fragments

Certain aspects relate to antibody fragments, such as antibody fragments that bind to antigen. The term functional antibody fragment includes antigen-binding fragments of an antibody that retain the ability to specifically bind to an antigen. These fragments are constituted of various arrangements of the variable region heavy chain (V_{H}) and/or light chain (V_{L}) and can include constant region heavy chain 1 (C_{H}1) and light chain (C_{L}). In some aspects, they lack the Fc region constituted of heavy chain 2 (C_{H}2) and 3 (C_{H}3) domains. Aspects of antigen binding fragments and the modifications thereof may include: (i) the Fab fragment type constituted with the V_{L}, V_{H}, C_{L}, and CHI domains; (ii) the Fd fragment type constituted with the V_{H} and CHI domains; (iii) the Fv fragment type constituted with the VI and V_{L} domains; (iv) the single domain fragment type, dAb, (Holt et al. Trends Biotechnol. 21 (11): 484-90 (2003)) constituted with a single V_{H} or V_{L} domain; (v) isolated complementarity determining region (CDR) regions. Such terms are described, for example, in Harlow and Lane, Antibodies: A Laboratory Manual, Cold Spring Harbor Laboratory, NY (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015), each of which are incorporated by reference in their entirety.

Antigen-binding fragments also include fragments of an antibody that retain exactly, at least, or at most 1, 2, or 3 CDRs from a light chain variable region. Fusions of CDR-containing sequences to an Fc region (or a C_{H}2 or C_{H}3 region thereof) are included within the scope of this definition including, for example, scFv fused, directly or indirectly, to an Fc region are included herein.

The term Fab fragment means a monovalent antigen-binding fragment of an antibody containing the variable (V_{L} and V_{H}) and the constant (C_{L} and C_{H}1) domains. The term Fab′ fragment means a monovalent antigen-binding fragment of a monoclonal antibody that is larger than a Fab fragment. For example, a Fab′ fragment includes the V_{L}, V_{H}, C_{L} and CHI domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two V_{H} and V_{L} domains and can further include all or part of the two C_{L} and C_{H}1 domains.

The term Fd fragment means a fragment of the heavy chain of a monoclonal antibody, which includes all or part of the V_{H}, including the CDRs. An Fd fragment can further include CHI region sequences.

The term Fv fragment means a monovalent antigen-binding fragment of a monoclonal antibody, including all or part of the V_{L} and V_{H}, and absent of the CI, and CHI domains. The V_{L} and V_{H} include, for example, the CDRs. Single-chain antibodies (sFv or scFv) are Fv molecules in which the V_{L} and V_{H} regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv) 2 means bivalent or bispecific sFv polypeptide chains that include oligomerization domains at their C-termini, separated from the sFv by a hinge region. The oligomerization domain comprises self-associating α-helices, e.g., leucine zippers, which can be further stabilized by additional disulfide bonds. (scFv) 2 fragments are also known as “miniantibodies” or “minibodies.”

A single domain antibody is an antigen-binding fragment containing only a V_{H} or the V_{L} domain. In some instances, two or more V_{H} regions are covalently joined with a peptide linker to create a bivalent domain antibody. The two V_{H} regions of a bivalent domain antibody may target the same or different antigens.

### 2. Fragment Crystallizable (Fc) Region

An Fc region contains two heavy chain fragments comprising the C_{H}2 and C_{H}3 domains of an antibody. The two heavy chain fragments are held together by two or more disulfide bonds and by hydrophobic interactions of the C_{H}3 domains. The term “Fc polypeptide” as used herein includes native and mutein forms of polypeptides derived from the Fc region of an antibody. Truncated forms of such polypeptides containing a hinge region that promotes dimerization are included.

C. Polypeptides with Antibody CDRs & Scaffolding Domains that Display the CDRs

Antigen-binding peptide scaffolds, such as CDRs, are used to generate protein-binding molecules in accordance with the aspects. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify. Skerra, J Mol Recognit, 13:167-87 (2000).

The protein scaffolds can be sourced from but are not limited to fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibiters of neuronal nitric oxide synthase (PIN) may also be used.

### D. Antibody Binding

The term “selective-binding agent”, “antigen-binding agent”, or “antigen-binding protein” refers to a molecule that binds to an antigen. Non-limiting examples include antibodies, antigen-binding fragments, scFv, Fab, Fab′, F(ab′) 2, single chain antibodies, peptides, peptide fragments and proteins.

The term “binding” refers to a direct association between two molecules, due to, for example, covalent, electrostatic, hydrophobic, and ionic and/or hydrogen-bond interactions, including interactions such as salt bridges and water bridges. “Immunologically reactive” means that the selective binding agent or antibody of interest will bind with antigens present in a biological sample. The term “immune complex” refers the combination formed when an antibody or selective binding agent binds to an epitope on an antigen.

### 1. Affinity/Avidity

The term “affinity” refers the strength with which an antibody or selective binding agent binds an epitope. In antibody binding reactions, this is expressed as the affinity constant (Kₐ or kₐ sometimes referred to as the association constant) for any given antibody or selective binding agent. Affinity is measured as a comparison of the binding strength of the antibody to its antigen relative to the binding strength of the antibody to an unrelated amino acid sequence. The terms “immunoreactive” and “preferentially binds” are used interchangeably herein with respect to antibodies and/or selective binding agent.

There are several experimental methods that can be used by one skilled in the art to evaluate the binding affinity of any given antibody or selective binding agent for its antigen. This is generally done by measuring the equilibrium dissociation constant (K_{D} or K_{d}), using the equation K_{D})=k_{off}/kₒₙ=[A=][B]/[AB]. The term k_{off} is the rate of dissociation between the antibody and antigen per unit time and is related to the concentration of antibody and antigen present in solution in the unbound form at equilibrium. The term kₒₙ is the rate of antibody and antigen association per unit time and is related to the concentration of the bound antigen-antibody complex at equilibrium. The units used for measuring the K_{D} are mol/L (molarity, or M), or concentration. The Kₐ of an antibody is the inverse of the K_{D} and is determined by the equation Kₐ=1/K_{D}. Examples of some experimental methods that can be used to determine the K_{D} value are enzyme-linked immunosorbent assays (ELISA), isothermal titration calorimetry (ITC), fluorescence anisotropy, surface plasmon resonance (SPR), and affinity capillary electrophoresis (ACE).

Antibodies deemed useful in certain aspects may have an equilibrium dissociation constant of at least, at most, exactly, or between (inclusive or exclusive) any two of about 10⁻⁶, 10⁻⁷, 10⁻⁸, 10⁻⁹, 10⁻¹⁰ M, 10⁻¹¹ M, 10⁻¹² M, or any range derivable therein. These values are reported for antibodies discussed herein and the same assay may be used to evaluate the binding properties of such antibodies. An antibody of the disclosure is said to “specifically bind” its target antigen when the dissociation constant (K_{D})) is about 10⁻⁸ M. The antibody specifically binds antigen with “high affinity” when the K_{D} is about 5× 10⁻⁹ M, and with “very high affinity” when the K_{D} is about 5×10⁻¹² M.

### 2. Epitope Specificity

The epitope of an antigen is the specific region of the antigen for which an antibody has binding affinity. In the case of protein or polypeptide antigens, the epitope is the specific residues (or specified amino acids or protein segment) that the antibody binds with high affinity. An antibody does not necessarily contact every residue within the protein. Nor does every single amino acid substitution or deletion within a protein necessarily affect binding affinity. For purposes of this specification and the accompanying claims, the terms “epitope” and “antigenic determinant” are used interchangeably to refer to the site on an antigen to which B and/or T cell receptors respond or recognize. Polypeptide epitopes can be formed from both contiguous amino acids and noncontiguous amino acids juxtaposed by tertiary folding of a polypeptide. In some aspects, an epitope includes at least 3, for example 3, 4, 5, 6, 7, 8, 9, or 10 amino acids, in a unique spatial conformation.

Epitope specificity of an antibody can be determined in a variety of ways. One approach, for example, involves testing a collection of overlapping peptides of about 15 amino acids spanning the full sequence of the protein and differing in increments of a small number of amino acids (e.g., 3 to 30 amino acids). The peptides are immobilized in separate wells of a microtiter dish. Immobilization can be accomplished, for example, by biotinylating one terminus of the peptides. This process may affect the antibody affinity for the epitope, therefore different samples of the same peptide can be biotinylated at the N and C terminus and immobilized in separate wells for the purposes of comparison. This is useful for identifying end-specific antibodies. Optionally, additional peptides can be included terminating at a particular amino acid of interest. This approach is useful for identifying end-specific antibodies to internal fragments. An antibody or antigen-binding fragment is screened for binding to each of the various peptides. The epitope is defined as a segment of amino acids that is common to all peptides to which the antibody shows high affinity binding.

It also is possible to determine without undue experimentation, whether an antibody has the same specificity as the antibody of this disclosure by determining whether the antibody being tested prevents an antibody of this disclosure from binding the protein or polypeptide with which the antibody is normally reactive. If the antibody being tested competes with the antibody of the disclosure as shown by a decrease in binding by the monoclonal antibody of this disclosure, then it is likely that the two antibodies bind to the same or a closely related epitope. Alternatively, one can pre-incubate the antibody of this disclosure with a protein with which it is normally reactive, and determine if the antibody being tested is inhibited in its ability to bind the antigen. If the antibody being tested is inhibited then, likely, it has the same, or a closely related, epitopic specificity as the antibody of this disclosure.

### 3. Modification of Antigen-Binding Domains

It is understood that the antibodies of the present disclosure may be modified to yield variant antibodies. Variant antibodies are substantially identical to the antibody polypeptide sequences, or fragments thereof, and still bind the epitopes of the present disclosure. Polypeptide sequences are “substantially identical” when optimally aligned using such programs as Clustal Omega, IGBLAST, GAP, or BESTFIT using default gap weights, they share at least 80% sequence identity, at least 90% sequence identity, at least 95% sequence identity, at least 96% sequence identity, at least 97% sequence identity, at least 98% sequence identity, or at least 99% sequence identity or any range therein.

As discussed herein, minor variations in the amino acid sequences of antibodies or antigen-binding regions thereof are contemplated as being encompassed by the present disclosure, providing that the variations in the amino acid sequence maintain at least 75%, more preferably at least 80%, at least 90%, at least 95%, at least 96%, at least 97%, at least 98% and most preferably at least 99% sequence identity. In some aspects, conservative amino acid replacements are contemplated.

The variable region of the antibodies of the present disclosure can be modified by mutating amino acid residues within the V_{H} and/or V_{L} CDR 1, CDR 2 and/or CDR 3 regions to improve one or more binding properties (e.g., affinity) of the antibody. Mutations may be introduced by site-directed mutagenesis or PCR-mediated mutagenesis and the effect on antibody binding, or other functional property of interest, can be evaluated in appropriate in vitro or in vivo assays. Preferably conservative modifications are introduced and typically no more than one, two, three, four or five residues within a CDR region are altered. The mutations may be amino acid substitutions, additions or deletions.

Framework modifications can be made to the antibodies to decrease immunogenicity, for example, by “backmutating” one or more framework residues to the corresponding germline sequence.

In addition, the antibodies of the disclosure may be engineered to include modifications within the Fc region to alter one or more functional properties of the antibody, such as serum half-life, complement fixation, Fc receptor binding, and/or antigen-dependent cellular cytotoxicity. Such modifications include, but are not limited to, alterations of the number of cysteine residues in the hinge region to facilitate assembly of the light and heavy chains or to increase or decrease the stability of the antibody (U.S. Pat. No. 5,677,425) and amino acid mutations in the Fc hinge region to decrease the biological half-life of the antibody (U.S. Pat. No. 6,165,745).

Fragments or analogs of antibodies or immunoglobulin molecules can be readily prepared by those skilled in the art. Certain preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Standard methods to identify protein sequences that fold into a known three-dimensional structure are available to those skilled in the art; Dill and McCallum., Science 338:1042-1046 (2012). Several algorithms for predicting protein structures and the gene sequences that encode these have been developed, and many of these algorithms can be found at the National Center for Biotechnology Information (on the World Wide Web at ncbi.nlm.nih.gov/guide/proteins/) and at the Bioinformatics Resource Portal (on the World Wide Web at expasy.org/proteomics). Thus, the foregoing examples demonstrate that those of skill in the art can recognize sequence motifs and structural conformations that may be used to define structural and functional domains in accordance with the disclosure.

It is also contemplated that the antigen-binding domain may be multi-specific or multivalent by multimerizing the antigen-binding domain with V_{H} and V_{L} region pairs that bind either the same antigen (multi-valent) or a different antigen (multi-specific).

### E. Enzymatic or Chemical Modification of Antibodies

Additionally, the antibodies of the disclosure may be enzymatically or chemically modified to produce further derivatives of the antibodies and antigen binding fragments that are described herein. The term “antibody derivative” can include post-translational modification to the linear polypeptide sequence of the antibody or fragment. The derivatized antibody or fragment thereof may comprise any molecule or substance that imparts a desired property to the antibody or fragment.

The derivatized antibody can comprise, for example, a chemical post-translational modification, a detectable (or labeling) moiety (e.g., a radioactive, colorimetric, antigenic, or enzymatic molecule, or a detectable bead), a molecule that binds to another molecule (e.g., biotin or streptavidin), a therapeutic or diagnostic moiety (e.g., a radioactive, cytotoxic, or pharmaceutically active moiety), or a molecule that increases the suitability of the antibody for a particular use (e.g., administration to a subject, such as a human subject, or other in vivo or in vitro uses). An antibody or fragment thereof can be covalently attached to a molecule or substance, such as a labeling moiety or a therapeutic moiety; covalent attachment does not prevent the antibody from generating an anti-idiotypic response. An antibody or fragment thereof can be non-covalently attached to a molecule or substance, such as a labeling moiety or a therapeutic moiety.

Optionally, an antibody or an antigen-binding fragment can be chemically conjugated to, or expressed as, a fusion protein with other proteins. In some aspects, polypeptides may be chemically modified by conjugating or fusing the polypeptide to serum protein, such as human serum albumin, to increase half-life of the resulting molecule. See, e.g., EP 0322094 and EP 0486525. In some aspects, the polypeptides may be conjugated to a diagnostic agent and used diagnostically, for example, to monitor the development or progression of a disease and determine the efficacy of a given treatment regimen. In some aspects, the polypeptides may also be conjugated to a therapeutic agent to provide a therapy in combination with the therapeutic effect of the polypeptide.

In some aspects, disclosed are antibodies and antibody-like molecules that are linked to at least one agent to form an antibody conjugate or payload. To increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules include toxins, therapeutic enzymes, antibiotics, radiolabeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles, or ligands.

### 1. Post-Translational Modifications

The antigen-binding protein can have or lack one or more post-translational modifications such as myristoylation, palmitoylation, isoprenylation or prenylation, farnesylation, geranylgeranylation, glypiation, acylation, acetylation, formylation, alkylation, methylation, amide bond formation, amidation at C-terminus, arginylation, polyglutamylation, polyglycylation, butyrylation, glycosylation, glycation, polysialylation, malonylation, hydroxylation, iodination, phosphorylation, adenylylation, propionylation, S-glutathionylation, S-nitrosylation, S-sulfenylation (aka S-sulphenylation), succinylation, sulfation, biotinylation, pegylation, SUMOylation, ubiquitination, neddylation, pupylation, disulfide bridges, or racemization. The antigen-binding protein can have reduced or increased amounts of one or more post-translational modifications as compared to the same antigen-binding protein expressed in the cell that is native to the encoded gene. The reduction or increase may be by at least or at most 25, 50, 60, 70, 80, 90, 100, 150, 200, 250, 300, 350, 400, 450, 500% or more (or any range derivable therein).

In some aspects, contemplated are glycosylation variants of antibodies, wherein the number and/or type of glycosylation site(s) has been altered compared to the amino acid sequences of the parent polypeptide. Glycosylation of the polypeptides can be altered, for example, by modifying one or more sites of glycosylation within the polypeptide sequence to increase the affinity of the polypeptide for antigen (U.S. Pat. Nos. 5,714,350 and 6,350,861, incorporated herein by reference). Antibody protein variants comprise a greater or a lesser number of N-linked glycosylation sites than the native antibody. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions that eliminate or alter this sequence will prevent addition of an N-linked carbohydrate chain present in the native polypeptide. For example, the glycosylation can be reduced by the deletion of an Asn or by substituting the Asn with a different amino acid. In other aspects, one or more new N-linked glycosylation sites are created.

Additional antibody variants include cysteine variants, wherein one or more cysteine residues in the parent or native amino acid sequence are deleted from or substituted with another amino acid (e.g., serine). Cysteine variants are useful, inter alia, when antibodies must be refolded into a biologically active conformation. Cysteine variants may have fewer cysteine residues than the native antibody and typically have an even number to minimize interactions resulting from unpaired cysteines.

In some aspects, the polypeptides can be pegylated to increase biological half-life by reacting the polypeptide with polyethylene glycol (PEG) or a reactive ester or aldehyde derivative of PEG, under conditions in which one or more PEG groups become attached to the polypeptide. Polypeptide pegylation may be carried out by an acylation reaction or an alkylation reaction with a reactive PEG molecule (or an analogous reactive water-soluble polymer). Methods for pegylating proteins are known in the art and can be applied to the polypeptides of the disclosure to obtain PEGylated derivatives of antibodies. See, e.g., EP 0154316 and EP 0401384, incorporated herein by reference. In some aspects, the antibody is conjugated or otherwise linked to transthyretin (TTR) or a TTR variant. The TTR or TTR variant can be chemically modified with, for example, a chemical selected from the group consisting of dextran, poly(n-vinyl pyrrolidone), polyethylene glycols, propropylene glycol homopolymers, polypropylene oxide/ethylene oxide co-polymers, polyoxyethylated polyols, and polyvinyl alcohols. As used herein, the term “polyethylene glycol” is intended to encompass any of the forms of PEG that have been used to derivatize other proteins.

### 2. Conjugates

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to be detected, and/or further quantified if desired. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. The label may be simply detected, or it may be quantified. A response that is simply detected generally comprises a response whose existence merely is confirmed, whereas a response that is quantified generally comprises a response having a quantifiable (e.g., numerically reportable) value such as an intensity, polarization, and/or other property. Examples of detectable labels include, but not limited to, radioactive isotopes, fluorescers, semiconductor nanocrystals, chemiluminescers, chromophores, enzymes, enzyme substrates, enzyme cofactors, enzyme inhibitors, dyes, metal ions, metal sols, ligands (e.g., biotin, streptavidin or haptens) and the like. Examples of labels are, but not limited to, horseradish peroxidase (HRP), fluorescein, fluorescein isothiocyanate (FITC), rhodamine, dansyl, umbelliferone, dimethyl acridinium ester (DMAE), Texas red, luminol, nicotinamide adenine dinucleotide phosphate (NADPH), and α- or β-galactosidase.

In luminescence or fluorescence assays, the detectable response may be generated directly using a luminophore or fluorophore associated with an assay component involved in binding, or indirectly using a luminophore or fluorophore associated with another (e.g., reporter or indicator) component. Examples of luminescent labels that produce signals include but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6.sup.th ed.). Examples of luminescent probes include, but are not limited to, aequorin and luciferases. Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6.sup.th ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/antibodies, e.g., rhodamine/anti-rhodamine, biotin/avidin and biotin/streptavidin.

Antibodies may also be coupled to low molecular weight haptens to increase the sensitivity of the antibody in an assay. The haptens can then be specifically detected by means of a second reaction. For example, it is common to use haptens such as biotin, which reacts avidin, or dinitrophenol, pyridoxal, and fluorescein, which can react with specific anti-hapten antibodies. See, Harlow and Lane (1988) supra.

Antibody conjugates also include those intended primarily for use in vitro, where the antibody is linked to a secondary binding ligand and/or to an enzyme to generate a colored product upon contact with a chromogenic substrate. Examples of suitable enzymes include, but are not limited to, urease, alkaline phosphatase, (horseradish) hydrogen peroxidase, or glucose oxidase. Preferred secondary binding ligands are biotin and/or avidin and streptavidin compounds. The uses of such labels are well known to those of skill in the art and are described, for example, in U.S. Pat. Nos. 3,817,837; 3,850,752; 3,939,350; 3,996,345; 4,277,437; 4,275,149; and 4,366,241; each incorporated herein by reference. Molecules containing azido groups may also be used to form covalent bonds to proteins through reactive nitrene intermediates that are generated by low intensity ultraviolet light.

Additional suitable conjugated molecules include ribonuclease (RNase), DNase I, an antisense oligonucleotide, an inhibitory RNA molecule such as a siRNA molecule, an immunostimulatory nucleic acid, aptamers, ribozymes, triplex forming molecules, and external guide sequences (e.g., guide RNAs). Aptamers are small nucleic acids ranging from 15-50 bases in length that fold into defined secondary and tertiary structures, such as stem-loops or G-quartets, and can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Ribozymes are nucleic acid molecules that can catalyze a chemical reaction, either intramolecularly or intermolecularly. Ribozymes typically cleave nucleic acid substrates through recognition and binding of the target substrate with subsequent cleavage. Triplex forming function nucleic acid molecules can interact with double-stranded or single-stranded nucleic acid by forming a triplex, in which three strands of DNA form a complex dependent on both Watson-Crick and Hoogsteen base-pairing. Triplex molecules can bind target regions with high affinity and specificity. The functional nucleic acid molecules may act as effectors, inhibitors, modulators, and stimulators of a specific activity possessed by a target molecule, or the functional nucleic acid molecules may possess a de novo activity independent of any other molecules.

The antibodies of the disclosure or antigen-binding regions thereof can also be linked to another functional molecule such as another antibody or ligand for a receptor to generate a bi-specific or multi-specific molecule that binds to at least two or more different binding sites or target molecules. Linking of the antibody to one or more other binding molecules, such as another antibody, antibody fragment, peptide, or binding mimetic, can be done, for example, by chemical coupling, genetic fusion, or noncovalent association. Multi-specific molecules can further include a third binding specificity, in addition to the first and second target epitope.

The antibodies or fragments thereof of the present disclosure may be linked to a moiety that is toxic to a cell to which the antibody is bound to form “depleting” antibodies.

The antibodies of the disclosure may also be attached to solid supports, which are particularly useful for immunoassays or purification of the target antigen. Such solid supports include, but are not limited to, glass, cellulose, polyacrylamide, nylon, polystyrene, polyvinyl chloride, or polypropylene.

The antibodies also can be bound to many different carriers. Thus, this disclosure also provides compositions containing the antibodies and another substance, active or inert. Examples of well-known carriers include glass, polystyrene, polypropylene, polyethylene, dextran, nylon, amylase, natural and modified cellulose, polyacrylamide, agarose, and magnetite. The nature of the carrier can be either soluble or insoluble for purposes of the disclosure. Those skilled in the art will know of other suitable carriers for binding monoclonal antibodies, or will be able to ascertain such, using routine experimentation.

In some aspects, contemplated are immunoconjugates comprising an antibody or antigen-binding fragment thereof conjugated (e.g., covalently attached) to a cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). In this way, the agent of interest can be targeted directly to cells bearing the targeted cell surface antigen. The antibody and agent may be associated through non-covalent interactions such as through electrostatic forces, or by covalent bonds. Various linkers, known in the art, can be employed to form the immunoconjugate. Additionally, the immunoconjugate can be provided in the form of a genetic fusion protein. In one aspect, an antibody may be conjugated to various therapeutic substances to target the cell surface antigen. Examples of conjugated agents include, but are not limited to, metal chelate complexes, drugs, toxins, and other effector molecules, such as cytokines, lymphokines, chemokines, immunomodulators, radiosensitizers, asparaginase, carboranes, and radioactive halogens.

In antibody drug conjugates (ADC), an antibody is conjugated to one or more drug moieties through a linker. The ADC may be prepared by several routes, employing organic chemistry reactions, conditions, and reagents known to those skilled in the art, including: (1) reaction of a nucleophilic group of an antibody with a bivalent linker reagent, to form antibody-L, via a covalent bond, followed by reaction with a drug moiety D; and (2) reaction of a nucleophilic group of a drug moiety with a bivalent linker reagent, to form D-L, via a covalent bond, followed by reaction with the nucleophilic group of an antibody. ADC may also be produced by modification of the antibody to introduce electrophilic moieties, which can react with nucleophilic substituents on the linker reagent or drug. Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

Examples of ADC known to a person skilled in the art are pro-drugs useful for the local delivery of cytotoxic or cytostatic agents, i.e. drugs to kill or inhibit tumor cells in the treatment of cancer (Syrigos and Epenetos, Anticancer Res. 19:605-614 (1999); Niculescu-Duvaz and Springer, Adv. Drg. Del. Rev. 26:151-172 (1997); U.S. Pat. No. 4,975,278). In contrast, systematic administration of these unconjugated drug agents may result in unacceptable levels of toxicity to normal cells as well as the target tumor cells (Baldwin et al., Lancet 1:603-5 (1986); Thorpe, “Antibody Carriers of Cytotoxic Agents in Cancer Therapy: A Review,” In: Monoclonal Antibodies '84: Biological and Clinical Applications, A. Pincera et al., (eds.) pp. 475-506) (1985). Both polyclonal antibodies and monoclonal antibodies have been reported as useful in these strategies (Rowland et al., Cancer Immunol. Immunother. 21:183-87 (1986)).

In certain aspects, ADCs include covalent or aggregative conjugates of antibodies, or antigen-binding fragments thereof, with other proteins or peptides, such as by expression of recombinant fusion proteins comprising heterologous polypeptides fused to the N-terminus or C-terminus of an antibody polypeptide. For example, the conjugated peptide may be a heterologous signal (or leader) polypeptide, e.g., the yeast alpha-factor leader, or a peptide such as an epitope tag (e.g., V5-His). Antibody-containing fusion proteins may comprise peptides added to facilitate purification or identification of the antibody (e.g., poly-His). An antibody polypeptide also can be linked to the FLAG® (Sigma-Aldrich, St. Louis, Mo.) peptide as described in Hopp et al., Bio/Technology 6:1204 (1988), and U.S. Pat. No. 5,011,912.

The conjugated agents can be linked to the antibody directly or indirectly, using any of a large number of available methods. Several methods are known in the art for the attachment or conjugation of an antibody to its conjugate moiety (Amon et al., 1985; Hellstrom et al., 1987; Thorpe, 1985; Baldwin et al., 1985; Thorpe et al., 1982).

Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody (U.S. Pat. Nos. 4,472,509 and 4,938,948, each incorporated herein by reference).

Monoclonal antibodies may also be reacted with an enzyme in the presence of a coupling agent such as glutaraldehyde or periodate.

Conjugates may also be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl) hexanediamine), bis-diazonium derivatives (such as bis(p-diazoniumbenzoyl)-ethylenediamine), diisocyanates (such as toluene 2,6-diisocyanate), and bis-active fluorine compounds (such as 1,5-difluoro-2,4-dinitrobenzene).

In some aspects, derivatization of immunoglobulins by selectively introducing sulfhydryl groups in the Fc region of an immunoglobulin, using reaction conditions that do not alter the antibody combining site, are contemplated. Antibody conjugates produced according to this methodology are disclosed to exhibit improved longevity, specificity, and sensitivity (U.S. Pat. No. 5,196,066, incorporated herein by reference).

Site-specific attachment of effector or reporter molecules, wherein the reporter or effector molecule is conjugated to a carbohydrate residue in the Fc region has also been disclosed in the literature (O'Shannessy et al., J. Immunol. Methods 99 (2): 153-61 (1987)).

Bi-specific and multi-specific molecules can be prepared using methods known in the art. For example, each binding unit of the hi-specific molecule can be generated separately and then conjugated to one another. When the binding molecules are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitroberizoic acid) (DTNB), o-phenylenedimaleimide (ORDM), N-succinimidyl-3-(2-pyridyldithio) propionate (SPDP), and sulfosuccinimidyl 4-(N-maleimidomethyl) cyclohaxane-I-carboxylate (sulfo-SMCC) (Karpovsky et al., 1984; Liu et al., 1985). When the binding molecules are antibodies, they can be conjugated by sulfhydryl bonding of the C-terminus hinge regions of the two heavy chains.

### V. Proteins

In certain aspects the size of a protein or polypeptide (wild-type or modified) may comprise, but is not limited to, at least, at most, exactly, or between (inclusive or exclusive) any two of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids (e.g., amino acid substitutions) or be at least, at most, exactly, or between (inclusive or exclusive) any two of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, at most, exactly, or between (inclusive or exclusive) any two of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, or more contiguous amino acids, or any range derivable therein, of any of SEQ ID NOs: 1-20.

In some aspects, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, or more (or any derivable range therein) of any of SEQ ID NOs: 1-20.

In some aspects, the protein or polypeptide may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, or more (or any derivable range therein) contiguous amino acids of any of SEQ ID NOs: 1-20.

In some aspects, the polypeptide or protein may comprise at least, at most, exactly, or between (inclusive or exclusive) any two of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, or more (or any derivable range therein) contiguous amino acids that are at least, at most, exactly, or between (inclusive or exclusive) any two of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of any of SEQ ID NOs: 1-20.

In some aspects there is a polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 of any of SEQ ID NOs: 1-20 and comprising at least, at most, exactly, or between (inclusive or exclusive) any two of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 (or any derivable range therein) contiguous amino acids of any of SEQ ID NOs: 1-20.

Nucleotide as well as protein, polypeptide, and peptide sequences for various genes have been previously disclosed and may be found in the recognized computerized databases. Two commonly used databases are the National Center for Biotechnology Information's GENBANK® and GENPEPT® databases (on the World Wide Web at ncbi.nlm.nih.gov) and The Universal Protein Resource (UNIPROT®; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide per ml. The concentration of polypeptide in a composition can be at least, at most, exactly, or between (inclusive or exclusive) any two of about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

### A. Sequences

Amino acid sequences from 6 light chain variable region CDRs from the anti-TNFAIP2 antibody or a TNFAIP2 binding fragment thereof according to any one or more of the methods and compositions disclosed herein are provided in SEQ ID NOs: 9, 10, 11, 15, 16, and 17 as follows:

CDR-L1:
(SEQ ID NO: 9)
QSLLYSNGKTY
or
(SEQ ID NO: 15)
QNVGSI
CDR-L2:
(SEQ ID NO: 10)
LVS
or
(SEQ ID NO: 16)
SAS
CDR-L3:
(SEQ ID NO: 11)
VQGIHFPLT
or
(SEQ ID NO: 17)
QQYNSYPLT

Amino acid sequences from 6 heavy chain variable region CDRs from the anti-TNFAIP2 antibody or a TNFAIP2 binding fragment thereof according to any one or more of the methods and compositions disclosed herein are provided in SEQ ID NOs: 12, 13, 14, 18, 19, and 20 as follows:

CDR-H1:
(SEQ ID NO: 12)
GFSLTGYG
or
(SEQ ID NO: 18)
GFSLTSYG
CDR-H2:
(SEQ ID NO: 13)
IWGDGST
or
(SEQ ID NO: 19)
IWAGGSTN
CDR-H3:
(SEQ ID NO: 14)
AREVDY
or
(SEQ ID NO: 20)
ARDRASTMITPSFAY

Amino acid sequences from 2 light chains of TNFAIP2-binding proteins (e.g., antibodies) of the present disclosure are provided in SEQ ID NOs: 1 and 3.

(SEQ ID NO: 1)
DIVMTQSPLTLSVTIGQPASISCKSSQSLLYSNGKTYLNWLLQRPGQSPK
RLIYLVSKLDSGVPDRFTGSGSGTEFTLKISRVEAEDLGVYYCVQGIHFP
LTFGAGTKLELK
(SEQ ID NO: 3)
DIVMTQSQKFMSTSVGDRVSVTCKASQNVGSIVAWYQQKPGQSPKALIYS
ASYRYSGVPDRFTGSGSGTDFTLTISNVQSEDLAEYFCQQYNSYPLTFGG
GTKLEIK

Amino acid sequences from 2 heavy chains of TNFAIP2-binding proteins (e.g., antibodies) of the present disclosure are provided in SEQ ID NOs: 2 and 4.

(SEQ ID NO: 2)
EVQLEESGPGLVAPSQSLSITCTVSGFSLTGYGVNWVRQPPGKGLEWLGM
IWGDGSTDYNSALKSRLSIRKDNSKSQVFLKMNSLQTDDTARYYCAREVD
YWGQGTSVTVSS
(SEQ ID NO: 4)
EVQLEESGPGLVAPSQSLSITCTVSGFSLTSYGVHWVRQPPGKGLEWLGV
IWAGGSTNYNSALMSRLNISKDNSKSQVFLKMNSLQTDDTAMYYCARDRA
STMITPSFAYWGQGTLVTVSA

In some aspects, the V_{H} and the V_{L} are on the same polypeptide. In some aspects, the V_{H} and the V_{L} are on different polypeptides.

Aspects of the present disclosure include an anti-TNFAIP2 antibody or a TNFAIP2 binding fragment thereof according to any one or more of the methods and compositions disclosed herein comprising a light chain variable region (V_{L}) having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:1 or 3. Aspects of the present disclosure include antigen-binding proteins (e.g., antibodies, antibody-like molecules, or fragments thereof) (e.g., a TNFAIP2-binding protein, or an antibody or antigen-binding fragment thereof) comprising a heavy chain variable region (V_{H}) having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:2 or 4. In some aspects, the V_{L} has at least 85% identity to SEQ ID NO: 1 or 3. In some aspects, the V_{H} has at least 85% identity to SEQ ID NO:2 or 4. In some aspects, the V_{L} has at least 90% identity to SEQ ID NO: 1 or 3. In some aspects, the V_{H} has at least 90% identity to SEQ ID NO:2 or 4. In some aspects, the V_{L} has at least 95% identity to SEQ ID NO:1 or 3. In some aspects, the V_{H} has at least 95% identity to SEQ ID NO:2 or 4. In some aspects, the V_{L} comprises SEQ ID NO: 1 or 3. In some aspects, the V_{H} comprises SEQ ID NO:2 or 4.

In some aspects, the V_{L} has at least 85% identity to SEQ ID NO: 1 and the V_{H} has at least 85% identity to SEQ ID NO:2. In some aspects, the VI has at least 90% identity to SEQ ID NO: 1 and the V_{H} has at least 90% identity to SEQ ID NO:2. In some aspects, the V_{L} has at least 95% identity to SEQ ID NO:1 and the V_{H} has at least 95% identity to SEQ ID NO:2. In some aspects, the V_{L} comprises SEQ ID NO: 1 and the V_{H} comprises SEQ ID NO:2.

In some aspects, the V_{L} has at least 85% identity to SEQ ID NO:3 and the V_{H} has at least 85% identity to SEQ ID NO:4. In some aspects, the V_{L} has at least 90% identity to SEQ ID NO: 3 and the V_{H} has at least 90% identity to SEQ ID NO:4. In some aspects, the V_{L} has at least 95% identity to SEQ ID NO:3 and the V_{H} has at least 95% identity to SEQ ID NO:4. In some aspects, the V_{L} comprises SEQ ID NO:3 and the V_{H} comprises SEQ ID NO:4.

Aspects of the present disclosure include an anti-TNFAIP2 antibody or a TNFAIP2 binding fragment thereof according to any one or more of the methods and compositions disclosed herein comprising: a light chain variable region (V_{L}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:9, SEQ ID NO: 10, SEQ ID NO:11, SEQ ID NO: 15, SEQ ID NO: 16, or SEQ ID NO:17; and a heavy chain variable region (V_{H}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:12, SEQ ID NO:13, SEQ ID NO:14. SEQ ID NO:18, SEQ ID NO:19, or SEQ ID NO:20.

Some aspects are directed to an anti-TNFAIP2 antibody or a TNFAIP2 binding fragment thereof according to any one or more of the methods and compositions disclosed herein comprising: a light chain variable region (V_{L}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:9, SEQ ID NO: 10, and SEQ ID NO: 11; and a heavy chain variable region (V_{H}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:12, SEQ ID NO: 13, and SEQ ID NO: 14.

Some aspects are directed to an anti-TNFAIP2 antibody or a TNFAIP2 binding fragment thereof according to any one or more of the methods and compositions disclosed herein comprising: a light chain variable region (V_{L}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:15, SEQ ID NO:16, and SEQ ID NO: 17; and a heavy chain variable region (V_{H}) comprising three complementarity determining regions (CDRs), wherein one or more of the CDRs comprise an amino acid sequence having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:18, SEQ ID NO: 19, and SEQ ID NO: 20.

Some aspects are directed to an anti-TNFAIP2 antibody or a TNFAIP2 binding fragment thereof according to any one or more of the methods and compositions disclosed herein comprising: a CDR-L1 having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO: 9 or SEQ ID NO:15; a CDR-L2 having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO: 10 or SEQ ID NO:16; and a CDR-L3 having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO: 11 or SEQ ID NO:17; and a CDR-H1 having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:12 or SEQ ID NO:18; a CDR-H2 having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:13 or SEQ ID NO:19; and a CDR-H3 having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:14 or SEQ ID NO:20.

Some aspects are directed to an anti-TNFAIP2 antibody or a TNFAIP2 binding fragment thereof according to any one or more of the methods and compositions disclosed herein comprising a CDR-L1 having 85% sequence identity to SEQ ID NO:9 or SEQ ID NO: 15; a CDR-L2 having 85% sequence identity to SEQ ID NO:10 or SEQ ID NO:16; and a CDR-L3 having 85% sequence identity to SEQ ID NO:11 or SEQ ID NO: 17. Some aspects are directed to an anti-TNFAIP2 antibody or a TNFAIP2 binding fragment thereof according to any one or more of the methods and compositions disclosed herein comprising a CDR-L1 having 90% sequence identity to SEQ ID NO:9 or SEQ ID NO:15; a CDR-L2 having 90% sequence identity to SEQ ID NO: 10 or SEQ ID NO: 16; and a CDR-L3 having 90% sequence identity to SEQ ID NO:11 or SEQ ID NO: 17. Some aspects are directed to an anti-TNFAIP2 antibody or a TNFAIP2 binding fragment thereof according to any one or more of the methods and compositions disclosed herein comprising a CDR-L1 having 95% sequence identity to SEQ ID NO:9 or SEQ ID NO:15; a CDR-L2 having 95% sequence identity to SEQ ID NO:10 or SEQ ID NO: 16; and a CDR-L3 having 95% sequence identity to SEQ ID NO: 11 or SEQ ID NO: 17. Some aspects are directed to an anti-TNFAIP2 antibody or a TNFAIP2 binding fragment thereof according to any one or more of the methods and compositions disclosed herein comprising a CDR-L1 having 99% sequence identity to SEQ ID NO:9 or SEQ ID NO: 15; a CDR-L2 having 99% sequence identity to SEQ ID NO: 10 or SEQ ID NO: 16; and a CDR-L3 having 99% sequence identity to SEQ ID NO: 11 or SEQ ID NO: 17. Some aspects are directed to an anti-TNFAIP2 antibody or a TNFAIP2 binding fragment thereof according to any one or more of the methods and compositions disclosed herein comprising a CDR-L1 comprising SEQ ID NO:9 or SEQ ID NO:15; a CDR-L2 comprising SEQ ID NO:10 or SEQ ID NO: 16; and a CDR-L3 comprising SEQ ID NO:11 or SEQ ID NO:17. Some aspects are directed to an anti-TNFAIP2 antibody or a TNFAIP2 binding fragment thereof according to any one or more of the methods and compositions disclosed herein comprising a CDR-L1 consisting of SEQ ID NO:9 or SEQ ID NO: 15; a CDR-L2 consisting of SEQ ID NO: 10 or SEQ ID NO: 16; and a CDR-L3 consisting of SEQ ID NO:11 or SEQ ID NO:17.

Some aspects are directed to an anti-TNFAIP2 antibody or a TNFAIP2 binding fragment thereof according to any one or more of the methods and compositions disclosed herein comprising a CDR-L1 having 85% sequence identity to SEQ ID NO:9; a CDR-L2 having 85% sequence identity to SEQ ID NO:10; and a CDR-L3 having 85% sequence identity to SEQ ID NO:11. Some aspects are directed to an anti-TNFAIP2 antibody or a TNFAIP2 binding fragment thereof according to any one or more of the methods and compositions disclosed herein comprising a CDR-L1 having 90% sequence identity to SEQ ID NO:9; a CDR-L2 having 90% sequence identity to SEQ ID NO:10; and a CDR-L3 having 90% sequence identity to SEQ ID NO:11. Some aspects are directed to an anti-TNFAIP2 antibody or a TNFAIP2 binding fragment thereof according to any one or more of the methods and compositions disclosed herein comprising a CDR-L1 having 95% sequence identity to SEQ ID NO: 9; a CDR-L2 having 95% sequence identity to SEQ ID NO: 10; and a CDR-L3 having 95% sequence identity to SEQ ID NO:11. Some aspects are directed to an anti-TNFAIP2 antibody or a TNFAIP2 binding fragment thereof according to any one or more of the methods and compositions disclosed herein comprising a CDR-L1 having 99% sequence identity to SEQ ID NO: 9; a CDR-L2 having 99% sequence identity to SEQ ID NO: 10; and a CDR-L3 having 99% sequence identity to SEQ ID NO: 11. Some aspects are directed to an anti-TNFAIP2 antibody or a TNFAIP2 binding fragment thereof according to any one or more of the methods and compositions disclosed herein comprising a CDR-L1 comprising SEQ ID NO:9; a CDR-L2 comprising SEQ ID NO:10; and a CDR-L3 comprising SEQ ID NO:11. Some aspects are directed to an anti-TNFAIP2 antibody or a TNFAIP2 binding fragment thereof according to any one or more of the methods and compositions disclosed herein comprising a CDR-L1 consisting of SEQ ID NO:9; a CDR-L2 consisting of SEQ ID NO: 10; and a CDR-L3 consisting of SEQ ID NO:11.

Some aspects are directed to an anti-TNFAIP2 antibody or a TNFAIP2 binding fragment thereof according to any one or more of the methods and compositions disclosed herein comprising a CDR-L1 having 85% sequence identity to SEQ ID NO:15; a CDR-L2 having 85% sequence identity to SEQ ID NO: 16; and a CDR-L3 having 85% sequence identity to SEQ ID NO:17. Some aspects are directed to an anti-TNFAIP2 antibody or a TNFAIP2 binding fragment thereof according to any one or more of the methods and compositions disclosed herein comprising a CDR-L1 having 90% sequence identity to SEQ ID NO:15; a CDR-L2 having 90% sequence identity to SEQ ID NO:16; and a CDR-L3 having 90% sequence identity to SEQ ID NO: 17. Some aspects are directed to an anti-TNFAIP2 antibody or a TNFAIP2 binding fragment thereof according to any one or more of the methods and compositions disclosed herein comprising a CDR-L1 having 95% sequence identity to SEQ ID NO: 15; a CDR-L2 having 95% sequence identity to SEQ ID NO: 16; and a CDR-L3 having 95% sequence identity to SEQ ID NO:17. Some aspects are directed to an anti-TNFAIP2 antibody or a TNFAIP2 binding fragment thereof according to any one or more of the methods and compositions disclosed herein comprising a CDR-L1 having 99% sequence identity to SEQ ID NO:15; a CDR-L2 having 99% sequence identity to SEQ ID NO: 16; and a CDR-L3 having 99% sequence identity to SEQ ID NO:17. Some aspects are directed to an anti-TNFAIP2 antibody or a TNFAIP2 binding fragment thereof according to any one or more of the methods and compositions disclosed herein comprising a CDR-L1 comprising SEQ ID NO: 15; a CDR-L2 comprising SEQ ID NO:16; and a CDR-L3 comprising SEQ ID NO:17. Some aspects are directed to an anti-TNFAIP2 antibody or a TNFAIP2 binding fragment thereof according to any one or more of the methods and compositions disclosed herein comprising a CDR-L1 consisting of SEQ ID NO:15; a CDR-L2 consisting of SEQ ID NO:16; and a CDR-L3 consisting of SEQ ID NO:17.

Some aspects are directed to an anti-TNFAIP2 antibody or a TNFAIP2 binding fragment thereof according to any one or more of the methods and compositions disclosed herein comprising a CDR-H1 having 85% sequence identity to SEQ ID NO:12 or SEQ ID NO: 18; a CDR-H2 having 85% sequence identity to SEQ ID NO:13 or SEQ ID NO: 19; and a CDR-H3 having 85% sequence identity to SEQ ID NO:14 or SEQ ID NO:20. Some aspects are directed to an anti-TNFAIP2 antibody or a TNFAIP2 binding fragment thereof according to any one or more of the methods and compositions disclosed herein comprising a CDR-H1 having 90% sequence identity to SEQ ID NO:12 or SEQ ID NO:18; a CDR-H2 having 90% sequence identity to SEQ ID NO:13 or SEQ ID NO:19; and a CDR-H3 having 90% sequence identity to SEQ ID NO: 14 or SEQ ID NO:20. Some aspects are directed to an anti-TNFAIP2 antibody or a TNFAIP2 binding fragment thereof according to any one or more of the methods and compositions disclosed herein comprising a CDR-H1 having 95% sequence identity to SEQ ID NO: 12 or SEQ ID NO: 18; a CDR-H2 having 95% sequence identity to SEQ ID NO:13 or SEQ ID NO: 19; and a CDR-H3 having 95% sequence identity to SEQ ID NO: 14 or SEQ ID NO: 20. Some aspects are directed to an anti-TNFAIP2 antibody or a TNFAIP2 binding fragment thereof according to any one or more of the methods and compositions disclosed herein comprising a CDR-H1 having 99% sequence identity to SEQ ID NO:12 or SEQ ID NO: 18; a CDR-H2 having 99% sequence identity to SEQ ID NO: 13 or SEQ ID NO: 19; and a CDR-H3 having 99% sequence identity to SEQ ID NO:14 or SEQ ID NO:20. Some aspects are directed to an anti-TNFAIP2 antibody or a TNFAIP2 binding fragment thereof according to any one or more of the methods and compositions disclosed herein comprising a CDR-H1 comprising SEQ ID NO: 12 or SEQ ID NO: 18; a CDR-H2 comprising SEQ ID NO:13 or SEQ ID NO: 19; and a CDR-H3 comprising SEQ ID NO:14 or SEQ ID NO:20. Some aspects are directed to an anti-TNFAIP2 antibody or a TNFAIP2 binding fragment thereof according to any one or more of the methods and compositions disclosed herein comprising a CDR-H1 consisting of SEQ ID NO:12 or SEQ ID NO: 18; a CDR-H2 consisting of SEQ ID NO: 13 or SEQ ID NO: 19; and a CDR-H3 consisting of SEQ ID NO:14 or SEQ ID NO:20.

Some aspects are directed to an anti-TNFAIP2 antibody or a TNFAIP2 binding fragment thereof according to any one or more of the methods and compositions disclosed herein comprising a CDR-H1 having 85% sequence identity to SEQ ID NO:12; a CDR-H2 having 85% sequence identity to SEQ ID NO: 13; and a CDR-H3 having 85% sequence identity to SEQ ID NO:14. Some aspects are directed to an anti-TNFAIP2 antibody or a TNFAIP2 binding fragment thereof according to any one or more of the methods and compositions disclosed herein comprising a CDR-H1 having 90% sequence identity to SEQ ID NO:12; a CDR-H2 having 90% sequence identity to SEQ ID NO:13; and a CDR-H3 having 90% sequence identity to SEQ ID NO: 14. Some aspects are directed to an anti-TNFAIP2 antibody or a TNFAIP2 binding fragment thereof according to any one or more of the methods and compositions disclosed herein comprising a CDR-H1 having 95% sequence identity to SEQ ID NO: 12; a CDR-H2 having 95% sequence identity to SEQ ID NO: 13; and a CDR-H3 having 95% sequence identity to SEQ ID NO:14. Some aspects are directed to an anti-TNFAIP2 antibody or a TNFAIP2 binding fragment thereof according to any one or more of the methods and compositions disclosed herein comprising a CDR-H1 having 99% sequence identity to SEQ ID NO: 12; a CDR-H2 having 99% sequence identity to SEQ ID NO:13; and a CDR-H3 having 99% sequence identity to SEQ ID NO:14. Some aspects are directed to an anti-TNFAIP2 antibody or a TNFAIP2 binding fragment thereof according to any one or more of the methods and compositions disclosed herein comprising a CDR-H1 comprising SEQ ID NO: 12; a CDR-H2 comprising SEQ ID NO:13; and a CDR-H3 comprising SEQ ID NO:14. Some aspects are directed to an anti-TNFAIP2 antibody or a TNFAIP2 binding fragment thereof according to any one or more of the methods and compositions disclosed herein comprising a CDR-H1 consisting of SEQ ID NO: 12; a CDR-H2 consisting of SEQ ID NO: 13; and a CDR-H3 consisting of SEQ ID NO: 14.

Some aspects are directed to an anti-TNFAIP2 antibody or a TNFAIP2 binding fragment thereof according to any one or more of the methods and compositions disclosed herein comprising a CDR-H1 having 85% sequence identity to SEQ ID NO:18; a CDR-H2 having 85% sequence identity to SEQ ID NO: 19; and a CDR-H3 having 85% sequence identity to SEQ ID NO:20. Some aspects are directed to an anti-TNFAIP2 antibody or a TNFAIP2 binding fragment thereof according to any one or more of the methods and compositions disclosed herein comprising a CDR-H1 having 90% sequence identity to SEQ ID NO:18; a CDR-H2 having 90% sequence identity to SEQ ID NO:19; and a CDR-H3 having 90% sequence identity to SEQ ID NO:20. Some aspects are directed to an anti-TNFAIP2 antibody or a TNFAIP2 binding fragment thereof according to any one or more of the methods and compositions disclosed herein comprising a CDR-H1 having 95% sequence identity to SEQ ID NO: 18; a CDR-H2 having 95% sequence identity to SEQ ID NO: 19; and a CDR-H3 having 95% sequence identity to SEQ ID NO:20. Some aspects are directed to an anti-TNFAIP2 antibody or a TNFAIP2 binding fragment thereof according to any one or more of the methods and compositions disclosed herein comprising a CDR-H1 having 99% sequence identity to SEQ ID NO: 18; a CDR-H2 having 99% sequence identity to SEQ ID NO:19; and a CDR-H3 having 99% sequence identity to SEQ ID NO:20. Some aspects are directed to an anti-TNFAIP2 antibody or a TNFAIP2 binding fragment thereof according to any one or more of the methods and compositions disclosed herein comprising a CDR-H1 comprising SEQ ID NO: 18; a CDR-H2 comprising SEQ ID NO:19; and a CDR-H3 comprising SEQ ID NO:20. Some aspects are directed to an anti-TNFAIP2 antibody or a TNFAIP2 binding fragment thereof according to any one or more of the methods and compositions disclosed herein comprising a CDR-H1 consisting of SEQ ID NO: 18; a CDR-H2 consisting of SEQ ID NO: 19; and a CDR-H3 consisting of SEQ ID NO:20.

Some aspects are directed to an anti-TNFAIP2 antibody or a TNFAIP2 binding fragment thereof according to any one or more of the methods and compositions disclosed herein comprising a CDR-L1 having 85% sequence identity to SEQ ID NO:9; a CDR-L2 having 85% sequence identity to SEQ ID NO: 10; and a CDR-L3 having 85% sequence identity to SEQ ID NO: 11; and a CDR-H1 having 85% sequence identity to SEQ ID NO:12; a CDR-H2 having 85% sequence identity to SEQ ID NO:13; and a CDR-H3 having 85% sequence identity to SEQ ID NO: 14. Some aspects are directed to an anti-TNFAIP2 antibody or a TNFAIP2 binding fragment thereof according to any one or more of the methods and compositions disclosed herein comprising a CDR-L1 having 90% sequence identity to SEQ ID NO:9; a CDR-L2 having 90% sequence identity to SEQ ID NO: 10; and a CDR-L3 having 90% sequence identity to SEQ ID NO:11; and a CDR-H1 having 90% sequence identity to SEQ ID NO: 12; a CDR-H2 having 90% sequence identity to SEQ ID NO:13; and a CDR-H3 having 90% sequence identity to SEQ ID NO:14. Some aspects are directed to an anti-TNFAIP2 antibody or a TNFAIP2 binding fragment thereof according to any one or more of the methods and compositions disclosed herein comprising a CDR-L1 having 95% sequence identity to SEQ ID NO: 9; a CDR-L2 having 95% sequence identity to SEQ ID NO: 10; and a CDR-L3 having 95% sequence identity to SEQ ID NO: 11; and a CDR-H1 having 95% sequence identity to SEQ ID NO: 12; a CDR-H2 having 95% sequence identity to SEQ ID NO:13; and a CDR-H3 having 95% sequence identity to SEQ ID NO:14. Some aspects are directed to an anti-TNFAIP2 antibody or a TNFAIP2 binding fragment thereof according to any one or more of the methods and compositions disclosed herein comprising a CDR-L1 having 99% sequence identity to SEQ ID NO:9; a CDR-L2 having 99% sequence identity to SEQ ID NO:10; and a CDR-L3 having 99% sequence identity to SEQ ID NO: 11; and a CDR-H1 having 99% sequence identity to SEQ ID NO:12; a CDR-H2 having 99% sequence identity to SEQ ID NO:13; and a CDR-H3 having 99% sequence identity to SEQ ID NO:14. Some aspects are directed to an anti-TNFAIP2 antibody or a TNFAIP2 binding fragment thereof according to any one or more of the methods and compositions disclosed herein comprising a CDR-L1 comprising SEQ ID NO: 9; a CDR-L2 comprising SEQ ID NO:10; and a CDR-L3 comprising SEQ ID NO:11; and a CDR-H1 comprising SEQ ID NO: 12; a CDR-H2 comprising SEQ ID NO: 13; and a CDR-H3 comprising SEQ ID NO:14. Some aspects are directed to an anti-TNFAIP2 antibody or a TNFAIP2 binding fragment thereof according to any one or more of the methods and compositions disclosed herein comprising a CDR-L1 consisting of SEQ ID NO:9; a CDR-L2 consisting of SEQ ID NO:10; and a CDR-L3 consisting of SEQ ID NO:11; and a CDR-H1 consisting of SEQ ID NO:12; a CDR-H2 consisting of SEQ ID NO:13; and a CDR-H3 consisting of SEQ ID NO: 14.

Some aspects are directed to an anti-TNFAIP2 antibody or a TNFAIP2 binding fragment thereof according to any one or more of the methods and compositions disclosed herein comprising a CDR-L1 having 85% sequence identity to SEQ ID NO:15; a CDR-L2 having 85% sequence identity to SEQ ID NO: 16; and a CDR-L3 having 85% sequence identity to SEQ ID NO:17; and a CDR-H1 having 85% sequence identity to SEQ ID NO:18; a CDR-H2 having 85% sequence identity to SEQ ID NO:19; and a CDR-H3 having 85% sequence identity to SEQ ID NO:20. Some aspects are directed to an anti-TNFAIP2 antibody or a TNFAIP2 binding fragment thereof according to any one or more of the methods and compositions disclosed herein comprising a CDR-L1 having 90% sequence identity to SEQ ID NO: 15; a CDR-L2 having 90% sequence identity to SEQ ID NO:16; and a CDR-L3 having 90% sequence identity to SEQ ID NO:17; and a CDR-H1 having 90% sequence identity to SEQ ID NO:18; a CDR-H2 having 90% sequence identity to SEQ ID NO: 19; and a CDR-H3 having 90% sequence identity to SEQ ID NO:20. Some aspects are directed to an anti-TNFAIP2 antibody or a TNFAIP2 binding fragment thereof according to any one or more of the methods and compositions disclosed herein comprising a CDR-L1 having 95% sequence identity to SEQ ID NO: 15; a CDR-L2 having 95% sequence identity to SEQ ID NO:16; and a CDR-L3 having 95% sequence identity to SEQ ID NO:17; and a CDR-H1 having 95% sequence identity to SEQ ID NO:18; a CDR-H2 having 95% sequence identity to SEQ ID NO: 19; and a CDR-H3 having 95% sequence identity to SEQ ID NO:20. Some aspects are directed to an anti-TNFAIP2 antibody or a TNFAIP2 binding fragment thereof according to any one or more of the methods and compositions disclosed herein comprising a CDR-L1 having 99% sequence identity to SEQ ID NO:15; a CDR-L2 having 99% sequence identity to SEQ ID NO:16; and a CDR-L3 having 99% sequence identity to SEQ ID NO:17; and a CDR-H1 having 99% sequence identity to SEQ ID NO:18; a CDR-H2 having 99% sequence identity to SEQ ID NO:19; and a CDR-H3 having 99% sequence identity to SEQ ID NO:20. Some aspects are directed to an anti-TNFAIP2 antibody or a TNFAIP2 binding fragment thereof according to any one or more of the methods and compositions disclosed herein comprising a CDR-L1 comprising SEQ ID NO:15; a CDR-L2 comprising SEQ ID NO:16; and a CDR-L3 comprising SEQ ID NO: 17; and a CDR-H1 comprising SEQ ID NO:18; a CDR-H2 comprising SEQ ID NO:19; and a CDR-H3 comprising SEQ ID NO:20. Some aspects are directed to an anti-TNFAIP2 antibody or a TNFAIP2 binding fragment thereof according to any one or more of the methods and compositions disclosed herein comprising a CDR-L1 consisting of SEQ ID NO: 15; a CDR-L2 consisting of SEQ ID NO:16; and a CDR-L3 consisting of SEQ ID NO:17; and a CDR-H1 consisting of SEQ ID NO:18; a CDR-H2 consisting of SEQ ID NO:19; and a CDR-H3 consisting of SEQ ID NO:20.

### VI. Nucleic Acids

Some aspects are directed to a nucleic acid encoding for a TNFAIP2-binding protein. Some aspects are directed to a nucleic acid encoding for a polypeptide or fragment thereof that specifically binds TNFAIP2.

In some aspects, the nucleic acid encodes the heavy chain variable region and the light chain variable region of the TNFAIP2-binding protein. In some aspects, the nucleic acid encodes the light chain variable region of the TNFAIP2-binding protein. In some aspects, the nucleic acid encodes the heavy chain variable region of the TNFAIP2-binding protein. In some aspects, the nucleic acid encoding the light chain variable region comprises SEQ ID NO:5 or 7. In some aspects, the nucleic acid encoding the heavy chain variable region comprises SEQ ID NO: 6 or 8.

In certain aspects, there are polynucleotide variants having substantial identity to the sequences disclosed herein, e.g., those comprising at least, at most, exactly, or between (inclusive or exclusive) any two of 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 80%, at least 85%, at least 90%, or at least 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, at least, at most, exactly, or between (inclusive or exclusive) any two of 5, 10, 15, 20, 25, 30, 35, 40, 45, 50, 75, 100, 125, 175, 200, 250, 300, 350, 400, or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### A. Sequences

Nucleotide sequences from 2 light chains of TNFAIP2-binding proteins (e.g., antibodies) of the present disclosure are provided in SEQ ID NOs: 5 and 7.

(SEQ ID NO: 5)
GACATTGTGATGACCCAGTCTCCACTCACTTTGTCGGTTACCATTGGACA
ACCAGCCTCTATCTCTTGCAAGTCAAGTCAGAGCCTCTTATATAGTAATG
GAAAAACCTATTTGAATTGGTTATTACAGAGGCCAGGCCAGTCTCCAAAG
CGCCTAATCTATCTGGTGTCTAAACTGGACTCTGGAGTCCCTGACAGGTT
CACTGGCAGTGGATCAGGAACAGAATTTACACTGAAAATCAGCAGAGTGG
AGGCTGAGGATTTGGGAGTTTATTACTGCGTGCAAGGTATACATTTTCCG
CTCACGTTCGGTGCTGGGACCAAGCTGGAGCTGAAAC
(SEQ ID NO: 7)
GACATTGTGATGACCCAGTCTCAAAAATTCATGTCCACATCAGTAGGAGA
CAGGGTCAGCGTCACCTGCAAGGCCAGTCAGAATGTGGGTTCTATTGTAG
CCTGGTATCAACAGAAACCAGGGCAATCTCCTAAAGCACTGATTTACTCG
GCATCCTACCGGTACAGTGGAGTCCCTGATCGCTTCACAGGCAGTGGATC
TGGGACAGATTTCACTCTCACCATCAGCAATGTGCAGTCTGAAGACTTGG
CAGAGTATTTCTGTCAGCAATATAACAGCTATCCTCTGACGTTCGGTGGA
GGCACCAAGCTGGAAATCAAAC

Nucleotide sequences from 2 heavy chains of TNFAIP2-binding proteins (e.g., antibodies) of the present disclosure are provided in SEQ ID NOs: 6 and 8.

(SEQ ID NO: 6)
GAGGTGCAGCTGGAGGAGTCAGGACCTGGCCTGGTGGCGCCCTCACAGAG
CCTGTCCATCACATGCACCGTCTCAGGGTTCTCATTAACCGGCTATGGTG
TAAACTGGGTTCGCCAGCCTCCAGGAAAGGGTCTGGAGTGGCTGGGAATG
ATATGGGGTGATGGAAGCACAGACTATAATTCAGCTCTCAAATCCAGACT
GAGCATCAGGAAGGACAACTCCAAGAGCCAAGTTTTCTTAAAAATGAACA
GTCTGCAAACTGATGACACAGCCAGGTACTACTGTGCCAGAGAGGTGGAC
TACTGGGGTCAAGGAACCTCAGTCACCGTCTCCTCAG
(SEQ ID NO: 8)
GAGGTGCAGCTGGAGGAGTCAGGACCTGGCCTGGTGGCGCCCTCACAGAG
CCTGTCCATCACTTGCACTGTCTCTGGGTTTTCATTAACCAGCTATGGTG
TACACTGGGTTCGCCAGCCTCCAGGAAAGGGTCTGGAGTGGCTGGGAGTA
ATATGGGCTGGTGGAAGCACAAATTATAATTCGGCTCTTATGTCCAGACT
GAACATCAGCAAAGACAACTCCAAGAGCCAAGTTTTCTTAAAAATGAACA
GTCTGCAAACTGATGACACAGCCATGTACTACTGTGCCAGAGATCGGGCT
TCTACTATGATTACACCTTCGTTTGCTTACTGGGGCCAAGGGACTCTGGT
CACTGTCTCTGCAG

In some aspects, the V_{H} and the V_{L} are encoded by the same nucleotide sequence. In some aspects, the V_{H} and the V_{L} are encoded by different nucleotide sequences.

Aspects of the present disclosure include antigen-binding proteins (e.g., antibodies, antibody-like molecules, or fragments thereof) (e.g., a TNFAIP2-binding protein, or an antibody or antigen-binding fragment thereof) comprising a light chain variable region (V_{L}) having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:5 or 7. Aspects of the present disclosure include antigen-binding proteins (e.g., antibodies, antibody-like molecules, or fragments thereof) (e.g., a TNFAIP2-binding protein, or an antibody or antigen-binding fragment thereof) comprising a heavy chain variable region (V_{H}) having at least, at most, exactly, or between (inclusive or exclusive) any two of 85%, 86%, 87%, 88%, 89%, 90%, 92%, 93%, 94%, 95%, 96%, 97%, 98%, 99%, or 100% sequence identity, or any range or value derivable therein, to SEQ ID NO:6 or 8. In some aspects, the V_{L} has at least 85% identity to SEQ ID NO:5 or 7. In some aspects, the V_{H} has at least 85% identity to SEQ ID NO:6 or 8. In some aspects, the V_{L} has at least 90% identity to SEQ ID NO:5 or 7. In some aspects, the V_{H} has at least 90% identity to SEQ ID NO:6 or 8. In some aspects, the V_{L} has at least 95% identity to SEQ ID NO:5 or 7. In some aspects, the V_{H} has at least 95% identity to SEQ ID NO:6 or 8. In some aspects, the V_{L} comprises SEQ ID NO:5 or 7. In some aspects, the V_{H} comprises SEQ ID NO: 6 or 8.

In some aspects, the V_{L} has at least 85% identity to SEQ ID NO:5 and the V_{H} has at least 85% identity to SEQ ID NO:6. In some aspects, the V_{L} has at least 90% identity to SEQ ID NO: 5 and the V_{H} has at least 90% identity to SEQ ID NO:6. In some aspects, the V_{L} has at least 95% identity to SEQ ID NO:5 and the V_{H} has at least 95% identity to SEQ ID NO:6. In some aspects, the V_{L} comprises SEQ ID NO:5 and the VI comprises SEQ ID NO:6.

In some aspects, the V_{L} has at least 85% identity to SEQ ID NO:7 and the V_{H} has at least 85% identity to SEQ ID NO:8. In some aspects, the V_{L} has at least 90% identity to SEQ ID NO: 7 and the V_{H} has at least 90% identity to SEQ ID NO:8. In some aspects, the V_{L} has at least 95% identity to SEQ ID NO:7 and the V_{H} has at least 95% identity to SEQ ID NO:8. In some aspects, the V_{L} comprises SEQ ID NO:7 and the V_{H} comprises SEQ ID NO:8.

### VII. Antibody Production

Methods for preparing and characterizing antibodies for use in diagnostic and detection assays, for purification, and for use as therapeutics are well known in the art as disclosed in, for example, U.S. Pat. Nos. 4,011,308; 4,722,890; 4,016,043; 3,876,504; 3,770,380; and 4,372,745, each incorporated herein by reference (see, e.g., Harlow and Lane, Antibodies: A Laboratory Manual, Cold Spring Harbor Laboratory, 1988; incorporated herein by reference). These antibodies may be polyclonal or monoclonal antibody preparations, monospecific antisera, human antibodies, chimeric antibodies, such as humanized antibodies, altered antibodies, F(ab′)2 fragments, Fab fragments, Fv fragments, single-domain antibodies, dimeric or trimeric antibody fragment constructs, minibodies, or functional fragments thereof which bind to the antigen in question. In certain aspects, polypeptides, peptides, and proteins and immunogenic fragments thereof can also be synthesized in solution or on a solid support in accordance with conventional techniques.

In some aspects, there are nucleic acid molecules encoding antibody or antibody-like polypeptides (e.g., heavy or light chain, variable domain only, or full-length). These may be generated by methods known in the art, e.g., isolated from B cells of mice that have been immunized and isolated, phage display, expressed in any suitable recombinant expression system and allowed to assemble to form antibody molecules.

Unless specified otherwise, the antibodies can be isolated from any suitable biological source, e.g., murine, rat, rabbit, goat, camelid, sheep, or canine.

In an example, a polyclonal antibody is prepared by immunizing an animal with an antigen or a portion thereof and collecting antisera from that immunized animal. The antigen may be altered compared to an antigen sequence found in nature. A variant or altered antigenic peptide or polypeptide can be employed to generate antibodies. Inocula are typically prepared by dispersing the antigenic composition in a physiologically tolerable diluent to form an aqueous composition. Antisera is subsequently collected by methods known in the arts, and the serum may be used as-is for various applications or else the desired antibody fraction may be purified by well-known methods, such as affinity chromatography (Harlow and Lane, Antibodies: A Laboratory Manual, Cold Spring Harbor Laboratory, 1988).

Methods of making monoclonal antibodies are also well known in the art (e.g., U.S. Pat. No. 4,196,265, herein incorporated by reference in its entirety for all purposes). Typically, this technique involves immunizing a suitable animal with a selected immunogenic composition, e.g., a purified or partially purified protein, polypeptide, peptide, or domain. Resulting antibody-producing B-cells from the immunized animal, or all dissociated splenocytes, are then induced to fuse with cells from an immortalized cell line to form hybridomas. Myeloma cell lines suited for use in hybridoma-producing fusion procedures preferably are non-antibody-producing and have high fusion efficiency and enzyme deficiencies that render then incapable of growing in certain selective media that support the growth of only the desired fused cells (hybridomas). Typically, the fusion partner includes a property that allows selection of the resulting hybridomas using specific media. For example, fusion partners can be hypoxanthine/aminopterin/thymidine (HAT)-sensitive.

Methods for generating hybrids of antibody-producing spleen or lymph node cells and myeloma cells usually comprise mixing somatic cells with myeloma cells in the presence of an agent or agents (chemical or electrical) that promote the fusion of cell membranes. Next, selection of hybridomas can be performed by culturing the cells by single-clone dilution in microtiter plates, followed by testing the individual clonal supernatants (after about two to three weeks) for the desired reactivity. Fusion procedures for making hybridomas, immunization protocols, and techniques for isolation of immunized splenocytes for fusion are known in the art.

For example, a hybridoma is produced by fusing a suitable immortal cell line (e.g., a myeloma cell line such as, but not limited to, Sp2/0, Sp2/0-AG14, NSO, NS1, NS2, AE-1, L.5, P3X63Ag8,653, Sp2 SA3, Sp2 MAI, Sp2 SS1, Sp2 SA5, U397, MIA 144, ACT IV, MOLT4, DA-1, JURKAT, WEHI, K-562, COS, RAJI, NIH 313, HL-60, MLA 144, NAMAIWA, NEURO 2A, CHO, PerC.6, YB2/O) or the like, or heteromyelomas, fusion products thereof, or any cell or fusion cell derived there from, or any other suitable cell line as known in the art, with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. Antibody producing cells can also be obtained from the peripheral blood or, preferably the spleen or lymph nodes, of humans or other suitable animals that have been immunized with the antigen of interest. Any other suitable host cell can also be used for expressing-heterologous or endogenous nucleic acid encoding an antibody, specified fragment or variant thereof, of the present disclosure. The fused cells (hybridomas) or recombinant cells can be isolated using selective culture conditions or other suitable known methods, and cloned by limiting dilution or cell sorting, or other known methods.

Other techniques for producing monoclonal antibodies include the viral or oncogenic transformation of B-lymphocytes, a molecular cloning approach may be used to generate a nucleic acid or polypeptide, the selected lymphocyte antibody method (SLAM) (see, e.g., Babcook et al., Proc. Natl. Acad. Sci. USA 93:7843-7848 (1996), the preparation of combinatorial immunoglobulin phagemid libraries from RNA isolated from the spleen of the immunized animal and selection of phagemids expressing appropriate antibodies, or producing a cell expressing an antibody from a genomic sequence of the cell comprising a modified immunoglobulin locus using Cre-mediated site-specific recombination (see, e.g., U.S. Pat. No. 6,091,001).

Monoclonal antibodies may be further purified using filtration, centrifugation, and various chromatographic methods such as high-performance liquid chromatography (HPLC). Monoclonal antibodies may be further screened or optimized for properties relating to specificity, avidity, half-life, immunogenicity, binding association, binding disassociation, or overall functional properties relative to being a treatment for infection. Thus, monoclonal antibodies may have alterations in the amino acid sequence of CDRs, including insertions, deletions, or substitutions with a conserved or non-conserved amino acid.

Chimeric, humanized, or primatized antibodies of the present disclosure can be prepared based on the sequence of a reference monoclonal antibody prepared using standard molecular biology techniques. DNA encoding the heavy and light chain immunoglobulins can be obtained from the hybridoma of interest and engineered to contain non-reference (e.g., human) immunoglobulin sequences using standard molecular biology techniques. For example, to create a chimeric antibody, the murine variable regions can be linked to human constant regions using methods known in the art (U.S. Pat. No. 4,816,567). To create a humanized antibody, the murine CDR regions can be inserted into a human framework using methods known in the art (U.S. Pat. Nos. 5,225,539 and 5,530,101; 5,585,089; 5,693,762 and 6,180,370). Similarly, to create a primatized antibody, the murine CDR regions can be inserted into a primate framework using methods known in the art (WO 93/02108 and WO 99/55369).

Techniques for making partially to fully human antibodies are known in the art and any such techniques can be used. According to one aspect, fully human antibody sequences are made in a transgenic mouse which has been engineered to express human heavy and light chain antibody genes. Multiple strains of such transgenic mice have been made which can produce different classes of antibodies. B cells from transgenic mice which are producing a desirable antibody can be fused to make hybridoma cell lines for continuous production of the desired antibody. (See for example, Russel et al., 2000; Gallo et al., 2000; Green, 1999; Yang et al., 1999A; Yang, 1999B; Jakobovits, 1998; Green and Jakobovits, 1998; Jakobovits, 1998; Tsuda et al., 1997; Sherman-Gold, 1997; Mendez et al., 1997; Jakobovits, 1996; Jakobovits, 1995; Mendez et al., 1995; Jakobovits, 1994; Arbones et al., 1994; Jakobovits, 1993; Jakobovits et al., 1993; U.S. Pat. No. 6,075,181).

Alternatively, the antibodies of this disclosure can also be modified to create veneered antibodies. Veneered antibodies are those in which the exterior amino acid residues of the antibody of one species are judiciously replaced or “veneered” with those of a second species so that the antibodies of the first species will not be immunogenic in the second species thereby reducing the immunogenicity of the antibody. Since the antigenicity of a protein is primarily dependent on the nature of its surface, the immunogenicity of an antibody could be reduced by replacing the exposed residues which differ from those usually found in another mammalian species antibodies. This judicious replacement of exterior residues should have little, or no, effect on the interior domains, or on the interdomain contacts. Thus, ligand binding properties should be unaffected because of alterations which are limited to the variable region framework residues. The process is referred to as “veneering” since only the outer surface or skin of the antibody is altered, the supporting residues remain undisturbed.

The procedure for “veneering” makes use of the available sequence data for human antibody variable domains compiled by Kabat et al. (1987) Sequences of Proteins of Immunological Interest, 4th ed., Bethesda, Md., National Institutes of Health, updates to this database, and other accessible U.S. and foreign databases (both nucleic acid and protein). Non-limiting examples of the methods used to generate veneered antibodies include EP 519596; U.S. Pat. No. 6,797,492; and described in Padlan et al., 1991.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, cDNA, or the like, display library; e.g., as available from various commercial vendors such as MorphoSys (Martinsreid/Planegg, Del.), BioInvent (Lund, Sweden), Affitech (Oslo, Norway) using methods known in the art. Art known methods are described in the patent literature some of which include U.S. Pat. Nos. 4,704,692; 5,723,323; 5,763,192; 5,814,476; 5,817,483; 5,824,514; 5,976,862. Alternative methods rely upon immunization of transgenic animals (e.g., SCID mice) (Nguyen et al., 1977; Sandhu et al., 1996); Eren et al., 1998), that can produce a repertoire of human antibodies, as known in the art and/or as described herein. Such techniques, include, but are not limited to, ribosome display (Wanes et al., 1997; Hanes et al., 1998); single cell antibody producing technologies (e,g., selected lymphocyte antibody method (“SLAM”) (U.S. Pat. No. 5,627,052, Wen et al., 1987; Babcook et al., 1996); gel microdroplet and flow cytometry (Powell et al., 1990; Gray et al., 1995; Kenny et al., 1995); B-cell selection (Steenbakkers et al., 1994).

The antibodies of this disclosure can be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

The immunogenicity of a particular immunogen composition can be enhanced using non-specific stimulators of the immune response, known as adjuvants. Adjuvants that may be used include, but are not limited to, interleukin-1 (IL-1), IL-2, IL-4, IL-7, IL-12, γ-interferon (INF-γ), granulocyte-macrophage colony-stimulating factor (GMCSF), Bacillus Calmette-Guérin (BCG), aluminum hydroxide, muramyl dipeptide (MDP) compounds, muramyl tripeptide phosphatidyl ethanolamine (MTP-PE), lipid A, and monophosphoryl lipid A (MPL). Exemplary adjuvants may include complete Freund's adjuvant (a non-specific stimulator of the immune response containing killed Mycobacterium tuberculosis), incomplete Freund's adjuvants, and/or aluminum hydroxide adjuvant. In addition to adjuvants, it may be desirable to co-administer biologic response modifiers (BRM), such as but not limited to, Cimetidine (CIM; 1200 mg/d) (Smith/Kline, PA); low-dose Cyclophosphamide (CYP; 300 mg/m2) (Johnson/Mead, NJ), cytokines such as INF-β, IL-2, or IL-12, or genes encoding proteins involved in immune helper functions, such as B7-1 (CD80) or B7-2 (CD86). A phage-display system can be used to expand antibody molecule populations in vitro. Saiki, et al., Nature 324:163 (1986); Scharf et al., Science 233:1076 (1986); U.S. Pat. Nos. 4,683,195 and 4,683,202; Yang et al., J Mol Biol. 254:392 (1995); Barbas, III et al., Methods: Comp. Meth Enzymol. (1995) 8:94; Barbas, III et al., Proc Natl Acad Sci USA 88:7978 (1991).

Antibody fragments that retain the ability to recognize the antigen of interest will also find use herein. Several antibody fragments are known in the art that comprise antigen-binding sites capable of exhibiting immunological binding properties of an intact antibody molecule and can be subsequently modified by methods known in the arts. Functional fragments, including only the variable regions of the heavy and light chains, can also be produced using standard techniques such as recombinant production or preferential proteolytic cleavage of immunoglobulin molecules. These fragments are known as Fv. See, e.g., Inbar et al., Proc. Nat. Acad. Sci. USA 69:2659-2662 (1972); Hochman et al., Biochem. 15:2706-2710 (1976); and Ehrlich et al., Biochem. 19:4091-4096 (1980).

Single-chain variable fragments (scFvs) may be prepared by fusing DNA encoding a peptide linker between DNAs encoding the two variable domain polypeptides (V_{L} and V_{H}). scFvs can form antigen-binding monomers, or they can form multimers (e.g., dimers, trimers, or tetramers), depending on the length of a flexible linker between the two variable domains (Kortt et al., Prot. Eng. 10:423 (1997); Kort et al., Biomol. Eng. 18:95-108 (2001)). By combining different V_{L}- and V_{H}-comprising polypeptides, one can form multimeric scFvs that bind to different epitopes (Kriangkum et al., Biomol. Eng. 18:31-40 (2001)). Antigen-binding fragments are typically produced by recombinant DNA methods known to those skilled in the art. Although the two domains of the Fv fragment, V_{L} and VII, are coded for by separate genes, they can be joined using recombinant methods by a synthetic linker that enables them to be made as a single chain polypeptide (known as single chain Fv (sFv or scFv); see e.g., Bird et al., Science 242:423-426 (1988); and Huston et al., Proc. Natl. Acad. Sci. USA 85:5879-5883 (1988). Design criteria include determining the appropriate length to span the distance between the C-terminus of one chain and the N-terminus of the other, wherein the linker is generally formed from small hydrophilic amino acid residues that do not tend to coil or form secondary structures. Suitable linkers generally comprise polypeptide chains of alternating sets of glycine and serine residues and may include glutamic acid and lysine residues inserted to enhance solubility. Antigen-binding fragments are screened for utility in the same manner as intact antibodies. Such fragments include those obtained by N-terminal and/or C-terminal deletions, where the remaining amino acid sequence is substantially identical to the corresponding positions in the naturally occurring sequence deduced, for example, from a full-length cDNA sequence.

Also contemplated herein are non-peptide compounds having properties analogous to those of a template peptide. These types of non-peptide compounds are termed “peptide mimetics” or “peptidomimetics”. Fauchere, J. Adv. Drug Res. 15:29 (1986); Veber and Freidinger TINS p. 392 (1985); and Evans et al., J. Med. Chem. 30:1229 (1987).

Also contemplated are “antibody like binding peptidomimetics” (ABiPs), which are peptide-like molecules that act as pared-down antibodies and have certain advantages of longer serum half-life as well as less cumbersome synthesis methods. These analogs can be peptides, non-peptides or combinations of peptide and non-peptide regions. Fauchere, Adv. Drug Res. 15:29 (1986); Veber and Freidiner, TINS p. 392 (1985); and Evans et al., J. Med. Chem. 30:1229 (1987), which are incorporated herein by reference in their entirety for any purpose. Peptide mimetics that are structurally similar to therapeutically useful peptides may be used to produce a similar therapeutic or prophylactic effect. Such compounds are often developed with the aid of computerized molecular modeling. Generally, peptidomimetics of the disclosure are proteins that are structurally similar to an antibody displaying a desired biological activity, such as the ability to bind a protein, but have one or more peptide linkages optionally replaced by a linkage selected from: —CH₂NH—, —CH₂S—, —CH₂—CH₂—, —CH—CH— (cis and trans), —COCH₂—, —CH(OH) CH₂—, and —CH₂SO— by methods well known in the art. Systematic substitution of one or more amino acids of a consensus sequence with a D-amino acid of the same type (e.g., D-lysine in place of L-lysine) may be used to generate more stable proteins. In addition, constrained peptides comprising a consensus sequence or a substantially identical consensus sequence variation may be generated by methods known in the art (Rizo and Gierasch, Ann. Rev. Biochem. 61:387 (1992), incorporated herein by reference), for example, by adding internal cysteine residues capable of forming intramolecular disulfide bridges which cyclize the peptide.

Once generated, a phage display library can be used to improve the immunological binding affinity of Fab molecules using known techniques. See, e.g., Figini et al., J. Mol. Biol. 239:68 (1994). The coding sequences for the heavy and light chain portions of the Fab molecules selected from the phage display library can be isolated or synthesized and cloned into any suitable vector or replicon for expression. Any suitable expression system can be used.

### A. Expression

Nucleic acid molecules may be used to express large quantities of recombinant antibodies or to produce chimeric antibodies, single chain antibodies, antigen-binding fragments, immunoadhesins, diabodies, bi-specific antibodies, mutated antibodies, and other antibody derivatives. If the nucleic acid molecules are derived from a non-human, non-transgenic animal, the nucleic acid molecules may be used for antibody humanization.

### 1. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, modified antibodies, antibody fragments, and/or probes thereof. In addition to control sequences that govern transcription and translation, vectors and expression vectors may contain nucleic acid sequences that serve other functions as well.

To express the antibodies, or antigen-binding fragments thereof, DNA encoding partial or full-length light and heavy chains are inserted into expression vectors such that the gene area is operatively linked to transcriptional and translational control sequences. In some aspects, a vector that encodes a functionally complete human C_{H} or C_{L} immunoglobulin sequence with appropriate restriction sites engineered so that any V_{H} or V_{L} sequence can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### 2. Expression Systems

Numerous expression systems exist that comprise at least a part or all of the expression vectors discussed above. Prokaryote- and/or eukaryote-based systems can be employed to produce nucleic acid sequences, or their cognate polypeptides, proteins, and peptides. Commercially and widely available systems include, but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art can express a vector to produce a nucleic acid sequence or its cognate polypeptide using an appropriate expression system.

### 3. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue, or an organism, as described herein or as would be known to one of ordinary skill in the art. Such methods include, but are not limited to, direct delivery of DNA such as by injection (U.S. Pat. No. 5,994,624,5,981,274, 5,945,100, 5,780,448, 5,736,524, 5,702,932, 5,656,610, 5,589,466 and 5,580,859, each incorporated herein by reference), including microinjection (U.S. Pat. No. 5,789,215, incorporated herein by reference); by electroporation (U.S. Pat. No. 5,384,253, incorporated herein by reference); by calcium phosphate precipitation; by using DEAE dextran followed by polyethylene glycol; by direct sonic loading; by liposome mediated transfection; by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); or by PEG-mediated transformation of protoplasts (U.S. Pat. Nos. 4,684,611 and 4,952,500, each incorporated herein by reference); by desiccation/inhibition mediated DNA uptake. Other methods include viral transduction, such as gene transfer by lentiviral or retroviral transduction.

### 4. Host Cells

In another aspect, contemplated are the use of host cells into which a polypeptide, nucleic acid, or recombinant expression vector has been introduced. Antibodies and antibody-like molecules can be expressed in a variety of cell types. An expression construct encoding an antibody can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. In certain aspects, the antibody expression construct can be placed under control of a promoter that is linked to immune cell (e.g., T cell) activation. Control of antibody expression allows immune cells, such as tumor-targeting immune cells, to sense their surroundings and perform real-time modulation of cytokine signaling, both in the T cells themselves and in surrounding endogenous immune cells. One of skill in the art would understand the conditions under which to incubate host cells to maintain them and to permit replication of a vector. Also understood and known are techniques and conditions that would allow large-scale production of vectors and their cognate polypeptides. Host cells which may be used to express antibodies and other antigen-binding proteins of the present disclosure include, for example, murine myeloma cells (e.g., NS0 cells, SP2/0-Ag14 cells), Chinese hamster ovary (CHO) cells, baby hamster kidney (BHK21) cells, human embryonic kidney 293 (HEK293) cells, fibrosarcoma HT-1080 cells, and PER.C6 cells. In some aspects, the cell is an immune cell. In some aspects, the immune cell is a T cell. In some aspects, the immune cell is a B cell.

For stable transfection of mammalian cells, it is known, depending upon the expression vector and transfection technique used, only a small fraction of cells may integrate the foreign DNA into their genome. To identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

In some aspects, the cells disclosed herein can be used in methods to generate the TNFAIP2-binding proteins of the disclosure. The methods can comprise culturing the cell under conditions sufficient to express a nucleic acid encoding for a TNFAIP2-binding protein in the cell. Also contemplated are methods for generating the TNFAIP2-binding proteins of the disclosure, the method comprising (a) providing a nucleic acid encoding for the polypeptide to a cell, and (b) subjecting the cell to conditions sufficient to express the polypeptide from the nucleic acid.

### B. Isolation

The nucleic acid molecule encoding either or both of the entire heavy and light chains of an antibody or the variable regions thereof may be obtained from any source that produces antibodies. Methods of isolating mRNA encoding an antibody are well known in the art. The sequences of human heavy and light chain constant region genes are also known in the art. Nucleic acid molecules encoding the full-length heavy and/or light chains may then be expressed in a cell into which they have been introduced and the antibody isolated.

### VIII. Sample Preparation

In certain aspects, methods involve obtaining a sample from a subject. The methods of obtaining a sample from a subject provided herein may include methods of biopsy such as fine needle aspiration, core needle biopsy, vacuum assisted biopsy, incisional biopsy, excisional biopsy, punch biopsy, shave biopsy or skin biopsy. In certain aspects, the sample is obtained from sources including, but not limited to, blood, serum, plasma, sweat, hair follicle, buccal tissue, tears, menses, feces, or saliva. In specific aspects, the sample is obtained from serum. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

In certain aspects the samples are obtained by biopsy. In other aspects the sample is obtained by swabbing, endoscopy, scraping, phlebotomy, or any other methods known in the art. In some cases, the sample may be obtained, stored, or transported using components of a kit of the present methods. In some cases, multiple samples, such as multiple plasma or serum samples may be obtained for diagnosis by the methods described herein. In other cases, multiple samples, such as one or more samples from one tissue type (for example arteries or related tissues) and one or more samples from another specimen (for example serum) may be obtained for diagnosis by the methods. Samples may be obtained at different times are stored and/or analyzed by different methods. For example, a sample may be obtained and analyzed by routine staining methods or any other cytological analysis methods.

In some aspects the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, blood draw, endoscopy, or phlebotomy. The method of needle aspiration may further include fine needle aspiration, core needle biopsy, vacuum assisted biopsy, or large core biopsy. In some aspects, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods.

In some aspects of the present methods, the biological sample is obtained from a subject directly, from a medical professional, from a third party, or from a kit provided by a molecular profiling business or a third party. In some cases, the biological sample may be obtained after the subject, a medical professional, or a third party acquires and sends the biological sample to a third party. In some cases, the third party may provide suitable containers, and excipients for storage and transport of the biological sample to the third party.

In some aspects of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. A sample suitable for use and obtainable from an OTC kit may include tissues, cells, nucleic acids, genes, gene fragments, expression products, gene expression products, or gene expression product fragments of an individual to be tested. Methods for determining sample suitability and/or adequacy are provided.

In some aspects, the subject may be referred to a specialist such as a cardiologist. The specialist may likewise obtain a biological sample for testing or refer the individual to a testing center or laboratory for submission of the biological sample. In some cases, the medical professional may refer the subject to a testing center or laboratory for submission of the biological sample. In other cases, the subject may provide the sample. In some cases, a molecular profiling business may obtain the sample.

### IX. Administration of Therapeutic Compositions

The therapy provided herein may comprise administration of a combination of therapeutic agents, such as a first atherosclerosis therapy (e.g., a TNFAIP2 inhibitor, a lifestyle modification, a medication, or a procedure or surgery) and a second atherosclerosis therapy (e.g., a TNFAIP2 inhibitor, a lifestyle modification, a medication, or a procedure or surgery). The therapies may be administered in any suitable manner known in the art. For example, the first and second atherosclerosis treatment may be administered sequentially (at different times) or concurrently (at the same time). In some aspects, the first and second atherosclerosis treatments are administered in a separate composition. In some aspects, the first and second atherosclerosis treatments are in the same composition. Administration can be chronic or intermittent, as deemed appropriate by the supervising practitioner, including in view of any change in any undesirable side effects.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed.

In cases where the subject is provided with two or more doses of the composition, the duration between the administrations should be sufficient to allow time for distribution and effect in the individual, and in specific aspects the duration between doses is at most, at least, equal to, or between any two of 1, 2, 3, 4, 5, 6, 7, or more days. In some cases, the duration between administrations is 1-24 hours, 1-7 days, 1-4 weeks, 1-12 months, or more, or any range derivable there between. In some aspects, one or more atherosclerosis treatments are administered prior to one or more additional atherosclerosis treatments. In some aspects, one or more atherosclerosis treatments are administered at least, at most, equal two, or between any two of 1, 2, 3, 5, 6, 12, 24 hours or 1, 2, 3, 4, 6, 7, 8, 9, 10, 11, 12, 13, 14 days or 1, 2, 3, 4, 5, 6, 7, or 8, 9, 10, 11, 12 weeks or 1, 2, 3, 4, 5, 6, 7, or 8, 9, 10, 11, 12 months (or any derivable range therein) prior to the one or more additional atherosclerosis treatments. In some aspects, one or more atherosclerosis treatments are administered after one or more additional atherosclerosis treatments. In some aspects, the one or more atherosclerosis treatments are administered at least, at most, equal two, or between any two of 1, 2, 3, 5, 6, 12, 24 hours or 1, 2, 3, 4, 6, 7, 8, 9, 10, 11, 12, 13, 14 days or 1, 2, 3, 4, 5, 6, 7, or 8, 9, 10, 11, 12 weeks or 1, 2, 3, 4, 5, 6, 7, or 8, 9, 10, 11, 12 months (or any derivable range therein) after one or more additional atherosclerosis treatments.

The therapeutic agents of the disclosure may be administered by the same route of administration or by different routes of administration. In some aspects, the therapy is administered intravenously, intramuscularly, subcutaneously, topically, orally, transdermally, intraperitoneally, intraorbitally, by implantation, by inhalation, intrathecally, intraventricularly, or intranasally. In some aspects, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose. It will be understood by those skilled in the art and made aware that dosage units of μg/kg or mg/kg of body weight can be converted and expressed in comparable concentration units of μg/ml or mM (blood levels). It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

Typically, compositions are administered in a manner compatible with the dosage formulation, and in such amount as will be therapeutically or prophylactically effective for the subject being treated. Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

### X. Pharmaceutical Compositions

In certain aspects, the compositions or agents for use in the methods, such as atherosclerosis therapies or treatments or biomarker modulators, are suitably contained in a pharmaceutically acceptable carrier. The carrier is non-toxic, biocompatible and is selected so as not to detrimentally affect the biological activity of the agent. The agents in some aspects of the disclosure may be formulated into preparations for local delivery (i.e. to a specific location of the body, such as skeletal muscle or other tissue) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices and the like.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose, any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal or topical. Topical administration may be particularly advantageous for the treatment of skin cancers, to prevent chemotherapy-induced alopecia or other dermal hyperproliferative disorder. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol is between about 0.01 ml and 0.5 ml.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### XI. Kits

Certain aspects of the present disclosure also concern kits containing compositions of the disclosure or compositions to implement methods of the disclosure. In some aspects, kits can be used to evaluate one or more biomarkers (e.g., TNFAIP2). In certain aspects, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules, antibodies, or inhibitors, or any value or range and combination derivable therein. In some aspects, there are kits for evaluating biomarker activity or level in a cell.

The kits may comprise suitably aliquoted compositions of the present disclosure. The components of the kits may be packaged either in aqueous media or in lyophilized form. The container means of the kits will generally include at least one vial, test tube, flask, bottle, syringe or other suitable container means, into which a component may be placed, and preferably, suitably aliquoted. Where there is more than one component in the kit, the kit also may generally contain a second, third or other additional container into which the additional components may be separately placed. However, various combinations of components may be comprised in a vial. The kits of the present disclosure also will typically include a means for containing the composition and any other reagent containers in close confinement for commercial sale. Such containers may include injection or blow-molded plastic containers into which the desired vials are retained.

Individual components may also be provided in a kit in concentrated amounts; in some aspects, a component is provided individually in the same concentration as it would be in a solution with other components. Concentrations of components may be provided as 1×, 2×, 5×, 10×, or 20× or more.

Kits for using probes, antibodies, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes antibodies that bind to such biomarkers as well as nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, antibodies, probes, and inhibitors are included in some kit aspects. In addition, a kit may include a sample that is a negative or positive control for methylation of one or more biomarkers.

It is contemplated that any method or composition described herein can be implemented with respect to any other method or composition described herein and that different aspects may be combined. The claims originally filed are contemplated to cover claims that are multiply dependent on any filed claim or combination of filed claims.

### EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Identification of TNFAIP2 as an Early Detection Marker for Atherosclerosis

Background. As the inner layer of blood vessels, vascular endothelial cells (ECs) are directly exposed to both biochemical substances and hemodynamic forces. Endothelial dysfunction precedes the development of atherosclerotic plaques (Widmer & Lerman, 2014). Shear forces generated by blood flow are the major mechanical stimuli on vascular endothelium. Different shear stresses are directly associated with the formation and distribution of atherosclerotic plaques (Souilhol et al., 2020). Disturbed flow (DF) in arterial bifurcations and curvature promotes plaque formation. Oppositely, laminar flow (LSS) in the strait regions is athero-protective (Lee & Chiu, 2019). YAP is a mechano-sensitive molecule that mediates DF-induced endothelial inflammation and plaque formation (Wang et al., 2016). Investigations in cancer cells showed that atherosclerosis risk factors such as high glucose (Peng et al., 2017), ox-LDL (Yu et al., 2012; Y. Zhao et al., 2014)) and cigarette smoking promoted YAP activation (Zhao et al., 2014), suggesting that YAP is likely a universal sensor for atherogenic risk factors.

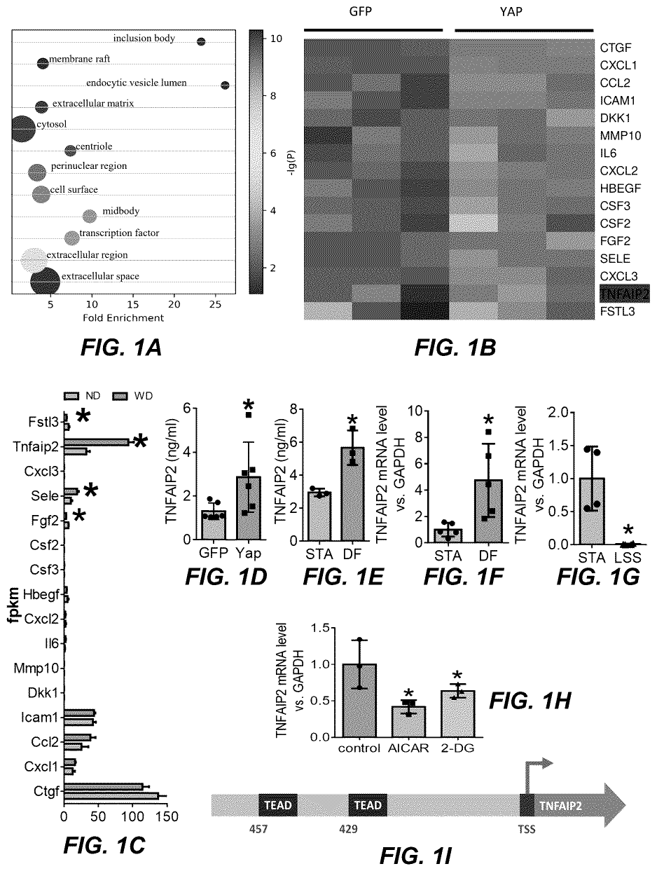

Identification of the secretory proteins controlled by YAP. The expression profile of HUVECs transiently transfected with constitutively active YAP (YAP S127A) was examined. Cell compartment enrichment revealed that 16 secretory proteins are induced by YAP overactivation in human endothelial cells (FIGS. 1A-1B). To confirm expression in vivo during the initiation of atherosclerosis, the gene expression profiling of isolated aortic endothelial cells from Apol mice fed on a western diet for eight weeks was investigated. Two genes (SELE and TNFAIP2) were significantly upregulated. TNFAIP2 was chosen for further study because it had a higher expression level (FIG. 1C).

The mechanism of TNFAIP2 regulation. To confirm that TNFAIP2 is under the control of YAP, the expression of TNFAIP2 was examined in HUVECs subjected to atheroprone or athero-protective stimuli. Consistent with findings that atheroprone DF promotes YAP activation, the expression of TNFAIP2 is induced by DF at both mRNA and protein levels (FIGS. 1D-1F). These results suggest that both YAP over-expression and YAP-activating DF promote expression of TNFAIP2. Oppositely, YAP inhibitory athero-protective stimuli, such as LSS, AICAR, and 2-DG, suppress the mRNA level of TNFAIP2 (FIGS. 1G-1H).

A TEAD-YAP interaction mediates most YAP-induced gene expression. Therefore, TEAD binding motifs were searched in the promoter region of TNFAIP2, and two TEAD-binding sequences were identified. Without wishing to be bound by theory, these data suggest that YAP-TEAD might regulate the expression of TNFAIP2 (FIG. 1I).

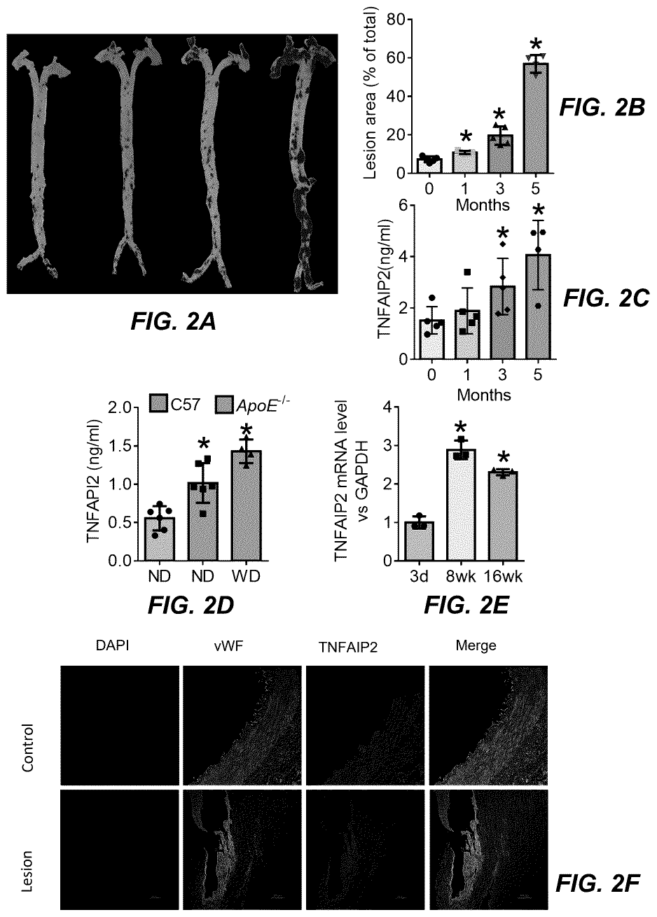

The serum TNFAIP2 level serves as an early marker of atherosclerosis. To identify whether the serum level of TNFAIP2 was increased at the early stage of atherosclerosis, serum concentration of TNFAIP2 was measured in ApoE^{-/-} mice fed on a western diet for different durations of time, reflecting different stages of atherosclerosis. The results showed that western diet treatment time-dependently increased the lesion area of ApoE^{-/-} mouse aortas (FIGS. 2A-2B). Moreover, the serum concentration of TNFAIP2 correlates with the expansion of the lesion area, suggesting that serum TNFAIP2 levels can predict the development of plaque formation (FIG. 2C).

To show whether the serum TNFAIP2 increases before the appearance of visible plaques, serum TNFAIP2 levels of wild-type mice and ApoE^{-/-} mice were compared with or without western diet-induced formation of plaques. As shown, wild-type mice displayed the lowest serum level of TNFAIP2, while the serum level of TNFAIP2 increased significantly in ApoE^{-/-} mice, even without observable western diet-induced formation of plaques (FIGS. 2D-2E), demonstrating the surprising and novel value of TNFAIP2 as an early diagnostic marker for atherosclerosis. To evaluate the clinical relevance of TNFAIP2 as an early diagnostic marker for atherosclerosis, TNFAIP2 expression was measured in the endothelium from clinical human samples with/without plaques. Consistent with the animal studies, the expression level of TNFAIP2 in vascular endothelium is higher in arteries with atherosclerotic plaques (FIG. 2F).

### Example 2—Antibody-Based TNFAIP2 Detection in Samples

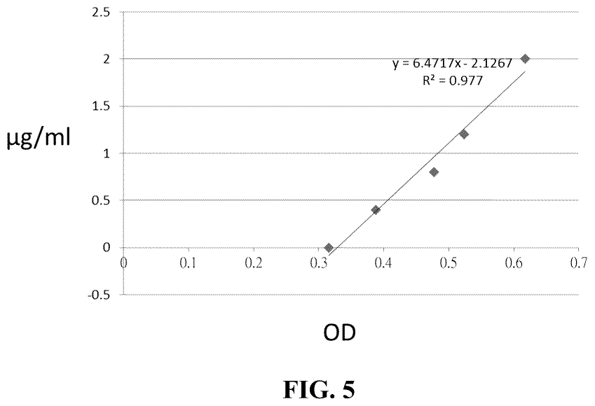

To investigate if the serum level of TNFAIP2 outperforms other prognostic biomarkers for atherosclerosis, recombinant TNFAIP2 protein was cloned and purified to produce a monoclonal antibody. An ELISA kit was developed based on the recombinant protein and monoclonal antibodies. The anti-TNFAIP2 antibodies included an antibody having a V_{L} chain having an amino acid sequence with at least 85% identity to SEQ ID NO: 1 and a VII chain having an amino acid sequence with at least 85% identity to SEQ ID NO:2 (clone 1), or an antibody having a V_{L} chain having an amino acid sequence with at least 85% identity to SEQ ID NO:3 and a V_{H} chain having an amino acid sequence with at least 85% identity to SEQ ID NO: 4 (clone 2), where clone 1 permitted TNFAIP2 capture, and clone 2 permitted detection of captured TNFAIP2. Results from an ELISA, shown in FIG. 5, demonstrate that TNFAIP2 can be detected in samples using the monoclonal antibodies produced.

### Example 3—the Role of TNFAIP2 in Atherogenesis

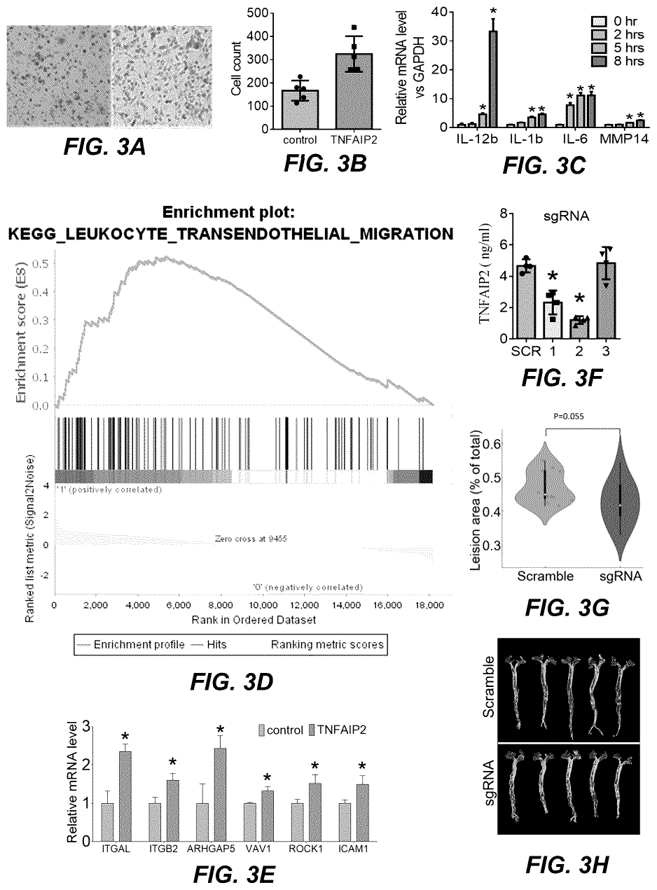

Effect of TNFAIP2 on circulating immune cells during atherogenesis. Monocyte adhesion, infiltration, and differentiation are important for initiation of atherosclerosis. In transwell migration assays, TNFAIP2 increases the infiltration of monocyte cells RAW246.7 (FIGS. 3A-3B). Quantitative RT-PCR further confirmed that genes related to inflammation and cell migration are induced by TNFAIP2 treatment (FIG. 3C). To explore the possible underlying mechanisms, the expression profile of monocyte cells RAW264.7 treated with TNFAIP2 was examined. Consistent with the transwell assay results, enrichment of the KEGG gene set revealed that the transendothelial migration pathway is activated by TNFAIP2 treatment (FIG. 3D). Quantitative RT-PCR further confirmed that TNFAIP2 treatment induces the expression of critical genes in the transendothelial migration pathway (FIG. 3E).

The effect of TNFAIP2 inhibition on atherogenesis. An adeno-associated virus (AAV) carrying a CRISPR/Cas9 system was constructed for TNFAIP2 knockdown in vivo. Three different sgRNAs targeting TNFAIP2 were constructed, and the AAVs were packaged. Knockdown efficiencies were tested in primary endothelial cells isolated from mouse aortas. The #2 sgRNA displayed the most robust knockdown efficiency and was selected for subsequent studies (FIG. 3F).

After being infected with AAV expressing the CRISPR/Cas9 for TNFAIP2 or scramble, ApoE^{-/-} mice were fed on western diet to facilitate the formation of atherosclerotic plaques. Five months later, these mice were sacrificed, and aortas were dissected and stained with Oil red O. Preliminary data revealed that TNFAIP2 knockdown suppresses plaque formation (p=0.055) (FIGS. 3G-3H).

### Example 4—the Role of Centrosomes in TNFAIP2-Induced Monocyte Transendothelial Migration

Background. The centrosome is regarded as the center of microtubule organization, responsible for chromosome separation during cell division. It has been recently revealed that centrosomes are also involved in cancer cell metastasis through controlling invadopodia formation, basal-cell extrusion, and promoting the secretion of pro-invasive factors. However, the role of the centrosome in transendothelial migration and atherogenesis is still unknown.

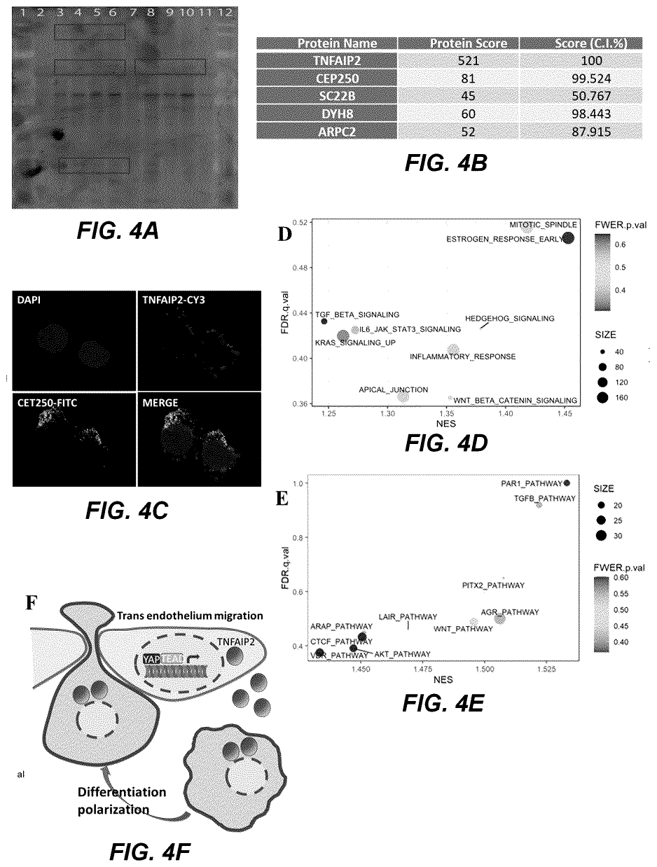

CEP250 interaction with TNFAIP2. Biotin proximity labeling was used to investigate the mechanism of TNFAIP2-induced transendothelial migration (FIG. 4A). CEP250, a protein critical for centrosome assembly, was identified (FIG. 4B), suggesting that centrosomes are likely involved in TNFAIP2-induced transendothelial migration and atherosclerosis. Immunostaining further confirmed that TNFAIP2 colocalizes with CEP250 in the prenuclear region (FIG. 4C).

The role of centrosomes on monocyte migration and atherogenesis. The centrosome is the organizer of the microtubule. Enrichment of the Hallmark gene set revealed that TNFAIP2 treatment activates the mitotic spindle pathway (FIG. 4D). Without wishing to be bound by theory, these data suggest that microtubule rearrangement might be involved in TNFAIP2-induced transendothelial migration. Enrichment of the BioCarta gene set revealed that the PAR1 pathway is activated by TNFAIP2 treatment (FIG. 4E). Since the PAR1 pathway has been reported to be involved in establishing cell polarity through regulating centrosome orientation, without wishing to be bound by theory, the PAR1 pathway may participate in TNFAIP2-induced cell polarized migration (FIG. 4F).

### Example 5—Exemplary Methods

Animals. Wild-type C57BL/6, ApoE^{-/-} mice were supplied by the Laboratory Animal Research Unit, City University of Hong Kong. Both male and female mice were used.

AAV-mediated gene knockdown. sgRNA sequences were designed by CCTop-CRISPR/Cas9 target online predictor. The sgRNA sequence was synthesized, annealed, and cloned into pX601-AAV-CMV: NLS-SaCas9-NLS-3xHA-bGHpA. The resultant plasmids were transferred with RGDLRVS-AAV9-cap, and pHelper into HEK 293T cells. Seventy-two hours post-transfection, HEK 293T cells were collected, lysed in AAV resuspension buffer, and precipitated with PEG at 4° C. The AAV were purified and concentrated with three rounds of hyperfiltration (100,000 MWCO, Sartorius). The AAV after purification was aliquoted and stored at −80° C.

Hemodynamic study. HUVECs were seeded on sterilized glass slides (75 mm×38 mm; Corning) pre-coated with fibronectin (50 μg/mL) at 5×10⁵ and cultured in EGM before being mounted to a customized flow chamber. The chamber was connected to the IBIDI flow system (IBIDI) to generate laminar shear (12 dyn/cm²), and disturbed shear force (0.5±4 dyn/cm² at 1 Hz), according to the manufacturer's instructions.

Isolation EC's mRNA from mouse aortas. Mice were sacrificed by overdose of anesthetics (Ketamine/Xylazine at 500/50 mg/kg IP), and perfused with PBS. The mouse aortas were dissected and flushed with 1 mL TRIZOL™ reagent (Thermo) over 1 min. The RNA was isolated according to the manufacturer's protocol. The purity of ECs was confirmed by the level of the ECs marker (eNOS) and SMC marker (σSMA).

Oil red O) staining. The mice were sacrificed by overdose of anesthetics. The aortas were dissected out and cut longitudinally to expose the inner lumen and pinned to a wax plate for overnight fixation in 4% paraformaldehyde solution at 4° C. On the following day, aortas were rinsed in water for 10 min, followed by a 60% isopropanol wash. After staining with Oil Red O solution with gentle shaking for 15 min, the samples were rinsed in 60% isopropanol again and then twice in water. The aortas were mounted onto a cover slide. The images were taken, and the lesion area will be quantitated by ImageJ.

RNA-sequencing. For YAP overexpression in human umbilical vein endothelial cells (HUVECs) and human aortic endothelial cells (HAECs), ECs were electroporated with 5 μg PCDNA3-YAP S127A and harvested 5 hours after electroporation. For NFAIP2 treatment, RAW cells were treated with recombinant TNFAIP2 (100 ng/ml) and harvested after 5 hours. The total RNA was extracted and sent to GENEWIZ (China) for sequencing.

Data analysis. Results are presented as means±SEM. For experiments involving two treatments, statistical significance is calculated by a two-tailed Student's t-test.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Brassington, K., Selemidis, S., Bozinovski, S., & VLahos, R. (2022). Chronic obstructive pulmonary disease and atherosclerosis: common mechanisms and novel therapeutics. Clinical Science, 136 (6), 405-423.
Deb, S., Felix, D. A., Koch, P., Deb, M. K., Szafranski, K., Buder, K., Sannai, M., Groth, M., Kirkpatrick, J., Pietsch, S., Gollowitzer, A., Groß, A., Riemenschneider, P., Koeberle, A., González-Estévez, C., & Rudolph, K. L. (2021). Tnfaip2/exoc3-driven lipid metabolism is essential for stem cell differentiation and organ homeostasis. EMBO Reports, 22 (1), e49328.
Gao, X., Tao, Y., Lamas, V., Huang, M., Yeh, W.-H., Pan, B., Hu, Y.-J., Hu, J. H., Thompson, D. B., Shu, Y., Li, Y., Wang, H., Yang, S., Xu, Q., Polley, D. B., Liberman, M. C., Kong, W.-J., Holt, J. R., Chen, Z.-Y., & Liu, D. R. (2018). Treatment of autosomal dominant hearing loss by in vivo delivery of genome editing agents. Nature, 553 (7687), 217-221.
Jia, L., Shi, Y., Wen, Y., Li, W., Feng, J., & Chen, C. (2018). The roles of TNFAIP2 in cancers and infectious diseases. Journal of Cellular and Molecular Medicine, 22 (11), 5188-5195.
Lee, D.-Y., & Chiu, J.-J. (2019). Atherosclerosis and flow: roles of epigenetic modulation in vascular endothelium. Journal of Biomedical Science, 26 (1), 56.
Lee, V. W. Y., Fong, F. Y. H., Cheng, F. W. T., & Yan, B. P. Y. (2019). Life's simple 7 and cardiovascular disease risk knowledge in Hong Kong. BMC Cardiovascular Disorders, 19 (1), 185.
Libby, P. (2021). The changing landscape of atherosclerosis. Nature, 592 (7855), 524-533.
LoMastro, G. M., & Holland, A. J. (2019). The Emerging Link between Centrosome Aberrations and Metastasis. Developmental Cell, 49 (3), 325-331.
Lusis, A. J. (2000). Atherosclerosis. Nature, 407 (6801), 233-41.
Peng, C., Zhu, Y., Zhang, W., Liao, Q., Chen, Y., Zhao, X., Guo, Q., Shen, P., Zhen, B., Qian, X., Yang, D., Zhang, J.-S., Xiao, D., Qin, W., & Pei, H. (2017). Regulation of the Hippo-YAP Pathway by Glucose Sensor O-GlcNAcylation. Molecular Cell, 68 (3), 591-604.e5.
Sarma, V., Wolf, F. W., Marks, R. M., Shows, T. B., & Dixit, V. M. (1992). Cloning of a novel tumor necrosis factor-alpha-inducible primary response gene that is differentially expressed in development and capillary tube-like formation in vitro. Journal of Immunology (Baltimore, Md.: 1950), 148 (10), 3302-3312.
Souilhol, C., Serbanovic-Canic, J., Fragiadaki, M., Chico, T. J., Ridger, V., Roddie, H., & Evans, P. C. (2020). Endothelial responses to shear stress in atherosclerosis: a novel role for developmental genes. Nature Reviews Cardiology, 17 (1), 52-63.
Stastna, M., & Eyk, J. E. V. (2012). Secreted proteins as a fundamental source for biomarker discovery. PROTEOMICS, 12 (4-5), 722-735.
Surma, S., Czober, T., Lepich, T., Sierka, O., & Bajor, G. (2020). Selected biomarkers of atherosclerosis-clinical aspects. Acta Angiologica, 26 (1), 28-39.
Tavakol, M., Ashraf, S., & Brener, S. J. (2012). Risks and Complications of Coronary Angiography: A Comprehensive Review. Global Journal of Health Science, 4 (1), 65-93.
Wang, L., Luo, J.-Y., Li, B., Tian, X. Y., Chen, L.-J., Huang, Y., Liu, J., Deng, D., Lau, C. W., Wan, S., Ai, D., Mak, K.-L. K., Tong, K. K., Kwan, K. M., Wang, N., Chiu, J.-J., Zhu, Y., & Huang, Y. (2016). Integrin-YAP/TAZ-JNK cascade mediates atheroprotective effect of unidirectional shear flow. Nature, 540 (7634), 579-582.
Widmer, R. J., & Lerman, A. (2014). Endothelial dysfunction and cardiovascular disease. Global Cardiology Science & Practice, 2014 (3), 291-308.
Yu, F.-X., Zhao, B., Panupinthu, N., Jewell, J. L., Lian, I., Wang, L. H., Zhao, J., Yuan, H., Tumaneng, K., Li, H., Fu, X.-D., Mills, G. B., & Guan, K.-L. (2012). Regulation of the Hippo-YAP Pathway by G-Protein-Coupled Receptor Signaling. Cell, 150 (4), 780-791.
Zhao, B., Ye, X., Yu, J., Li, L., Li, W., Li, S., Yu, J., Lin, J. D., Wang, C.-Y., Chinnaiyan, A. M., Lai, Z.-C., & Guan, K.-L. (2008). TEAD mediates YAP-dependent gene induction and growth control. 1. Genes & Development, 22 (14), 1962-1971. https://doi.org/10.1101/gad.1664408
Zhao, Y., Zhou, W., Xue, L., Zhang, W., & Zhan, Q. (2014). Nicotine Activates YAPI through nAChRs Mediated Signaling in Esophageal Squamous Cell Cancer (ESCC). PLOS ONE, 9 (3), e90836.

What is claimed is:

1. A method of measuring a TNFAIP2 level comprising:
obtaining a biological sample from a subject having, suspected of having, or at risk of having atherosclerosis; and
measuring the TNFAIP2 level in a biological sample from the subject as compared to a control.

2. The method of claim 1, wherein the biological sample comprises a serum, plasma, or tissue sample.

3. The method of claim 1, wherein measuring the TNFAIP2 level comprises using one or more antibodies that specifically bind TNFAIP2.

4. The method of claim 3, wherein the one or more antibodies that specifically bind TNFAIP2 comprise:
an antibody having:
a light chain variable region (V_{L}) comprising a CDR-L1 having at least 85% sequence identity to SEQ ID NO:9; a CDR-L2 having at least 85% sequence identity to SEQ ID NO:10; and a CDR-L3 having at least 85% sequence identity to SEQ ID NO:11; and
a heavy chain variable region (V_{H}) comprising a CDR-H1 having at least 85% sequence identity to SEQ ID NO:12, a CDR-H2 having at least 85% sequence identity to SEQ ID NO:13, and a CDR-L3 having at least 85% sequence identity to SEQ ID NO: 14; and/or
an antibody having:
a light chain variable region (V_{L}) comprising a CDR-L1 having at least 85% sequence identity to SEQ ID NO:15; a CDR-L2 having at least 85% sequence identity to SEQ ID NO: 16; and a CDR-L3 having at least 85% sequence identity to SEQ ID NO: 17; and
a heavy chain variable region (V_{H}) comprising a CDR-H1 having at least 85% sequence identity to SEQ ID NO:18, a CDR-H2 having at least 85% sequence identity to SEQ ID NO:19, and a CDR-L3 having at least 85% sequence identity to SEQ ID NO:20.

5. The method of claim 1, further comprising quantitating the TNFAIP2 level in the biological sample.

6. The method of claim 1, wherein the subject is one that:
has one or more symptoms of atherosclerosis; and/or
is being treated for atherosclerosis, will be treated for atherosclerosis, or is currently undergoing treatment for atherosclerosis.

7. The method of claim 1, wherein:
the control comprises a level of TNFAIP2 that is representative of a level of TNFAIP2 in a biological sample from a subject without atherosclerosis.

8. The method of claim 7, wherein:
TNFAIP2 is not detected in the sample or the measured TNFAIP2 level is not significantly different from or is less than the control, and
a treatment for atherosclerosis is not administered to the subject.

9. The method of claim 7, wherein:
the measured TNFAIP2 level is greater than the control, and
the method further comprises administering a treatment for atherosclerosis to the subject.

10. The method of claim 1, wherein:
the control comprises a level of TNFAIP2 that is representative of a level of TNFAIP2 in a biological sample from a subject with atherosclerosis.

11. The method of claim 10, wherein:
TNFAIP2 is not detected in the sample or the measured TNFAIP2 level is not significantly different from or is less than the control, and
a treatment for atherosclerosis is not administered to the subject.

12. The method of claim 10, wherein:
the measured TNFAIP2 level is not significantly different from or is greater than the control, and
the method further comprises administering a treatment for atherosclerosis to the subject.

13. A method for treating or preventing atherosclerosis in a subject, the method comprising administering an effective amount of a treatment for atherosclerosis to the subject after measuring the TNFAIP2 level in a biological sample from the subject as compared to a control.

14. The method of claim 13, wherein the biological sample comprises serum, plasma, or tissue sample.

15. The method of claim 13, wherein:
the control comprises a level of TNFAIP2 that is representative of a level of TNFAIP2 in a biological sample from a subject without atherosclerosis; and
TNFAIP2 is not detected in the sample or the measured TNFAIP2 level is not significantly different from or is less than the control, and a treatment for atherosclerosis is not administered to the subject; or
the measured TNFAIP2 level is greater than the control, and the method further comprises administering a treatment for atherosclerosis to the subject; or
the control comprises a level of TNFAIP2 that is representative of a level of TNFAIP2 in a biological sample from a subject with atherosclerosis; and
TNFAIP2 is not detected in the sample or the measured TNFAIP2 level is not significantly different from or is less than the control, and a treatment for atherosclerosis is not administered to the subject; or
the measured TNFAIP2 level is not significantly different from or is greater than the control, and the method further comprises administering a treatment for atherosclerosis to the subject.

16. The method of claim 13, wherein the treatment comprises a TNFAIP2 inhibitor, lifestyle changes, medications, procedures or surgeries, or a combination thereof.

17. The method of claim 16, wherein the TNFAIP2 inhibitor comprises an anti-TNFAIP2 antibody or a TNFAIP2 binding fragment thereof comprising:
an anti-TNFAIP2 antibody or a TNFAIP2 binding fragment thereof having:
a light chain variable region (V_{L}) comprising a CDR-L1 having at least 85% sequence identity to SEQ ID NO:9; a CDR-L2 having at least 85% sequence identity to SEQ ID NO: 10; and a CDR-L3 having at least 85% sequence identity to SEQ ID NO:11; and
a heavy chain variable region (V_{H}) comprising a CDR-H1 having at least 85% sequence identity to SEQ ID NO:12, a CDR-H2 having at least 85% sequence identity to SEQ ID NO:13, and a CDR-L3 having at least 85% sequence identity to SEQ ID NO: 14; and/or
an anti-TNFAIP2 antibody or a TNFAIP2 binding fragment thereof having:
a light chain variable region (V_{L}) comprising a CDR-L1 having at least 85% sequence identity to SEQ ID NO:15; a CDR-L2 having at least 85% sequence identity to SEQ ID NO:16; and a CDR-L3 having at least 85% sequence identity to SEQ ID NO:17; and
a heavy chain variable region (V_{H}) comprising a CDR-H1 having at least 85% sequence identity to SEQ ID NO:18, a CDR-H2 having at least 85% sequence identity to SEQ ID NO: 19, and a CDR-L3 having at least 85% sequence identity to SEQ ID NO:20.

18. A kit comprising one or more detection agents for measuring an expression level of TNFAIP2 in a sample from a subject.

19. The kit of claim 18, wherein the detection agent comprises an anti-TNFAIP2 antibody or a TNFAIP2 binding fragment thereof.

20. The kit of claim 18, wherein the anti-TNFAIP2 antibody or a TNFAIP2 binding fragment thereof comprises:
an anti-TNFAIP2 antibody or a TNFAIP2 binding fragment thereof having:
a light chain variable region (V_{L}) comprising a CDR-L1 having at least 85% sequence identity to SEQ ID NO:9; a CDR-L2 having at least 85% sequence identity to SEQ ID NO: 10; and a CDR-L3 having at least 85% sequence identity to SEQ ID NO:11; and
a heavy chain variable region (V_{H}) comprising a CDR-H1 having at least 85% sequence identity to SEQ ID NO:12, a CDR-H2 having at least 85% sequence identity to SEQ ID NO: 13, and a CDR-L3 having at least 85% sequence identity to SEQ ID NO: 14; and/or
an anti-TNFAIP2 antibody or a TNFAIP2 binding fragment thereof having:
a light chain variable region (V_{L}) comprising a CDR-L1 having at least 85% sequence identity to SEQ ID NO:15; a CDR-L2 having at least 85% sequence identity to SEQ ID NO:16; and a CDR-L3 having at least 85% sequence identity to SEQ ID NO:17; and
a heavy chain variable region (V_{H}) comprising a CDR-H1 having at least 85% sequence identity to SEQ ID NO:18, a CDR-H2 having at least 85% sequence identity to SEQ ID NO:19, and a CDR-L3 having at least 85% sequence identity to SEQ ID NO:20.


SEQUENCE LISTING (20 sequences)

SEQ ID NO: 1 - protein, 112 aa, synthetic construct
DIVMTQSPLTLSVTIGQPASISCKSSQSLLYSNGKTYLNWLLQRPGQSPKRLIYLVSKLDSGVPDRFTGSGSGTEFTLKISRVEAEDLGVYYCVQGIHFPLTFGAGTKLELK

SEQ ID NO: 2 - protein, 112 aa, synthetic construct
EVQLEESGPGLVAPSQSLSITCTVSGFSLTGYGVNWVRQPPGKGLEWLGMIWGDGSTDYNSALKSRLSIRKDNSKSQVFLKMNSLQTDDTARYYCAREVDYWGQGTSVTVSS

SEQ ID NO: 3 - protein, 107 aa, synthetic construct
DIVMTQSQKFMSTSVGDRVSVTCKASQNVGSIVAWYQQKPGQSPKALIYSASYRYSGVPDRFTGSGSGTDFTLTISNVQSEDLAEYFCQQYNSYPLTFGGGTKLEIK

SEQ ID NO: 4 - protein, 121 aa, synthetic construct
EVQLEESGPGLVAPSQSLSITCTVSGFSLTSYGVHWVRQPPGKGLEWLGVIWAGGSTNYNSALMSRLNISKDNSKSQVFLKMNSLQTDDTAMYYCARDRASTMITPSFAYWGQGTLVTVSA

SEQ ID NO: 5 - DNA, 337 bp, synthetic construct
  source 1..337: mol_type = other DNA
gacattgtgatgacccagtctccactcactttgtcggttaccattggacaaccagcctctatctcttgcaagtcaagtcagagcctcttatatagtaatggaaaaacctatttgaattggttattacagaggccaggccagtctccaaagcgcctaatctatctggtgtctaaactggactctggagtccctgacaggttcactggcagtggatcaggaacagaatttacactgaaaatcagcagagtggaggctgaggatttgggagtttattactgcgtgcaaggtatacattttccgctcacgttcggtgctgggaccaagctggagctgaaac

SEQ ID NO: 6 - DNA, 337 bp, synthetic construct
  source 1..337: mol_type = other DNA
gaggtgcagctggaggagtcaggacctggcctggtggcgccctcacagagcctgtccatcacatgcaccgtctcagggttctcattaaccggctatggtgtaaactgggttcgccagcctccaggaaagggtctggagtggctgggaatgatatggggtgatggaagcacagactataattcagctctcaaatccagactgagcatcaggaaggacaactccaagagccaagttttcttaaaaatgaacagtctgcaaactgatgacacagccaggtactactgtgccagagaggtggactactggggtcaaggaacctcagtcaccgtctcctcag

SEQ ID NO: 7 - DNA, 322 bp, synthetic construct
  source 1..322: mol_type = other DNA
gacattgtgatgacccagtctcaaaaattcatgtccacatcagtaggagacagggtcagcgtcacctgcaaggccagtcagaatgtgggttctattgtagcctggtatcaacagaaaccagggcaatctcctaaagcactgatttactcggcatcctaccggtacagtggagtccctgatcgcttcacaggcagtggatctgggacagatttcactctcaccatcagcaatgtgcagtctgaagacttggcagagtatttctgtcagcaatataacagctatcctctgacgttcggtggaggcaccaagctggaaatcaaac

SEQ ID NO: 8 - DNA, 364 bp, synthetic construct
  source 1..364: mol_type = other DNA
gaggtgcagctggaggagtcaggacctggcctggtggcgccctcacagagcctgtccatcacttgcactgtctctgggttttcattaaccagctatggtgtacactgggttcgccagcctccaggaaagggtctggagtggctgggagtaatatgggctggtggaagcacaaattataattcggctcttatgtccagactgaacatcagcaaagacaactccaagagccaagttttcttaaaaatgaacagtctgcaaactgatgacacagccatgtactactgtgccagagatcgggcttctactatgattacaccttcgtttgcttactggggccaagggactctggtcactgtctctgcag

SEQ ID NO: 9 - protein, 11 aa, synthetic construct
QSLLYSNGKTY

SEQ ID NO: 10 - skipped

SEQ ID NO: 11 - protein, 9 aa, synthetic construct
VQGIHFPLT

SEQ ID NO: 12 - protein, 8 aa, synthetic construct
GFSLTGYG

SEQ ID NO: 13 - protein, 7 aa, synthetic construct
IWGDGST

SEQ ID NO: 14 - protein, 6 aa, synthetic construct
AREVDY

SEQ ID NO: 15 - protein, 6 aa, synthetic construct
QNVGSI

SEQ ID NO: 16 - skipped

SEQ ID NO: 17 - protein, 9 aa, synthetic construct
QQYNSYPLT

SEQ ID NO: 18 - protein, 8 aa, synthetic construct
GFSLTSYG

SEQ ID NO: 19 - protein, 8 aa, synthetic construct
IWAGGSTN

SEQ ID NO: 20 - protein, 15 aa, synthetic construct
ARDRASTMITPSFAY